(19) United States
(12) Patent Application Publication
(10) Pub. No.: US 2011/0140043 A1
(43) Pub. Date: Jun. 16, 2011

(54) MATERIALS FOR ORGANIC ELECTROLUMINESCENCE DEVICE

(75) Inventors: Philipp Stoessel, Frankfurt (DE); Holger Heil, Frankfurt (DE); Dominik Joosten, Frankfurt (DE); Christof Pflumm, Frankfurt (DE); Anja Gerhard, Veitschöchheim (DE); Arne Buesing, Frankfurt (DE); Joachim Kaiser, Darmstadt (DE)

(73) Assignee: MERCK PATENT GMBH, Darmstadt (DE)

(21) Appl. No.: 13/001,305

(22) PCT Filed: Jun. 19, 2009
(86) PCT No.: PCT/EP2009/004448

§ 371 Date: Feb. 25, 2011

(30) Foreign Application Priority Data
Jul. 18, 2008 (DE) 10 2008 033 943.1

(51) Int. Cl. C09K 11/06; C07D 471/14; C07D 403/14; C07C 211/57; C07D 251/16; C07D 403/06; C07C 49/76; C07C 15/12

(52) U.S. Cl. 252/301.16; 546/82; 548/445; 564/336; 544/215; 548/305.4; 568/333; 568/332; 585/25

(57) ABSTRACT

The present invention relates to the compounds of the formula (1) and to organic electronic devices in which these compounds are used as matrix material in the emitting layer and/or as hole-transport material and/or as electron-blocking or exciton-blocking material and/or as electron-transport material.

The present invention relates to organic semiconductors and to the use thereof in organic electronic devices.

Organic semiconductors are being developed for a number of different electronic applications. The structure of organic electroluminescent devices (OLEDs) in which these organic semiconductors are employed as functional materials is described, for example, in U.S. Pat. No. 4,539,507, U.S. Pat. No. 5,151,629, EP 0676461 and WO 98/27136. However, further improvements are still desirable for use of these compounds in high-quality and long-lived displays. Thus, there is currently still a need for improvement, in particular, in the lifetime and efficiency of organic electroluminescent devices. Furthermore, it is necessary for the compounds to have high thermal stability and a high glass-transition temperature and to be capable of sublimation without decomposition.

Specifically also in the case of phosphorescent electroluminescent devices, significant improvements in the properties, in particular the lifetime, are still desirable.

There therefore continues to be a demand for improved materials, for example host materials, for fluorescent and phosphorescent emitters, but further improvements are also necessary in the case of charge-transport materials, i.e. hole- and electron-transport materials, and charge-blocking materials. The properties of these materials in particular are frequently responsible for the lifetime and efficiency of the organic electroluminescent device. There is still also a clear need for improvement, in particular, in the area of phosphorescent OLEDs.

Surprisingly, it has been found that benzophenone derivatives and diphenylmethane derivatives and corresponding heterocyclic derivatives which are substituted by selected substituents in the 3,5-position of the phenyl groups or the corresponding heterocyclic groups are very highly suitable for use in organic electroluminescent devices, where they result in significant improvements over the prior art. The present invention therefore relates to these compounds and to the use thereof in organic electronic devices. Depending on the substitution of the phenyl groups and depending on whether a benzophenone derivative or a diphenylmethane derivative is involved, the compounds according to the invention are particularly suitable as hole-transport materials, electron- or exciton-blocking materials, matrix materials for fluorescent or phosphorescent compounds, hole-blocking materials or electron-transport materials. The materials according to the invention enable a significant increase in the lifetime and a slight improvement in the efficiency of the organic electronic device compared with materials in accordance with the prior art. Furthermore, these cornpounds have high thermal stability.

WO 04/093207 discloses diarylketone derivatives as matrix materials for phosphorescent electroluminescent devices. Particularly preferred materials mentioned therein are keto compounds which are substituted by spirobifluorene. Benzophenone derivatives which are substituted in the 3,5-position of each of the phenyl groups, or the corresponding heterocyclic compounds, are not disclosed. However, it has been found that precisely this substitution pattern gives particularly good results on use in organic electronic devices.

The invention thus relates to compounds of the formula (1)

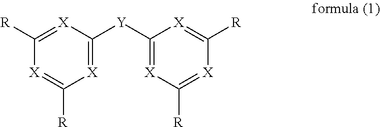

where the following applies to the symbols used:
Y is C═O or C(R¹)₂;
X is on each occurrence, identically or differently, CR² or N;
R is on each occurrence, identically or differently, an aromatic or heteroaromatic ring system having 5 to 60 aromatic ring atoms, which may be substituted by one or more radicals R³, or an N(Ar)₂, Si(Ar)₃, C(═O)Ar, OAr, ArSO, ArSO₂, P(Ar)₂, P(O)(Ar)₂ or B(Ar)₂ group;
Ar is on each occurrence, identically or differently, an aromatic or heteroaromatic ring system having 5 to 30 aromatic ring atoms, which may be substituted by one or more non-aromatic radicals R³; two radicals Ar here which are bonded to the same nitrogen, phosphorus or boron atom may also be linked to one another by a single bond or a bridge selected from B(R⁴), C(R⁴)₂, Si(R⁴)₂, C═O, C═NR⁴, C═C(R⁴)₂, O, S, S═O, SO₂, N(R⁴), P(R⁴) and P(═O)R⁴;
R¹ is on each occurrence, identically or differently, H, D, F or a linear alkyl group having 1 to 20 C atoms or a branched or cyclic alkyl group having 3 to 20 C atoms; a plurality of radicals R¹ here may form a ring system with one another;
R² is on each occurrence, identically or differently, H, D, F, CN, a straight-chain alkyl, alkoxy or thioalkoxy group having 1 to 40 C atoms or a branched or cyclic alkyl, alkoxy or thioalkoxy group having 3 to 40 C atoms, each of which may be substituted by one or more radicals R⁴, where one or more non-adjacent CH₂ groups may be replaced by R⁴C═CR⁴, C═C, O or S and where one or more H atoms may be replaced by F;
R³ is on each occurrence, identically or differently, H, D, F, Cl, Br, I, CHO, N(Ar)₂, C(═O)Ar, P(═O)(Ar)₂, S(═O)Ar, S(═O)₂Ar, CR²═CR²Ar, CN, NO₂, Si(R⁴)₃, B(OR⁴)₂, B(R⁴)₂, B(N(R⁴)₂)₂, OSO₂R⁴, a straight-chain alkyl, alkoxy or thioalkoxy group having 1 to 40 C atoms or a branched or cyclic alkyl, alkoxy or thioalkoxy group having 3 to 40 C atoms, each of which may be substituted by one or more radicals R⁴, where one or more non-adjacent CH₂ groups may be replaced by R⁴C═CR⁴, CEO, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴ and where one or more H atoms may be replaced by F, Cl, Br, I, CN or NO₂, or an aromatic or heteroaromatic ring system having 5 to 60 aromatic ring atoms, which may in each case be substituted by one or more radicals R⁴, or an aryloxy or heteroaryloxy group having 5 to 60 aromatic ring atoms, which may be substituted by one or more radicals R⁴, or a combination of these systems; two or more adjacent substituents R³ here may also form a mono- or polycyclic, aliphatic or aromatic ring system with one another;
R⁴ is on each occurrence, identically or differently, H, D or an aliphatic, aromatic and/or heteroaromatic hydrocarbon radical having 1 to 20 C atoms, in which, in addition, H atoms may be replaced by F; two or more adjacent substituents R⁴ here may also form a mono- or polycyclic, aliphatic or aromatic ring system with one another;
   the following compounds are excluded from the invention:

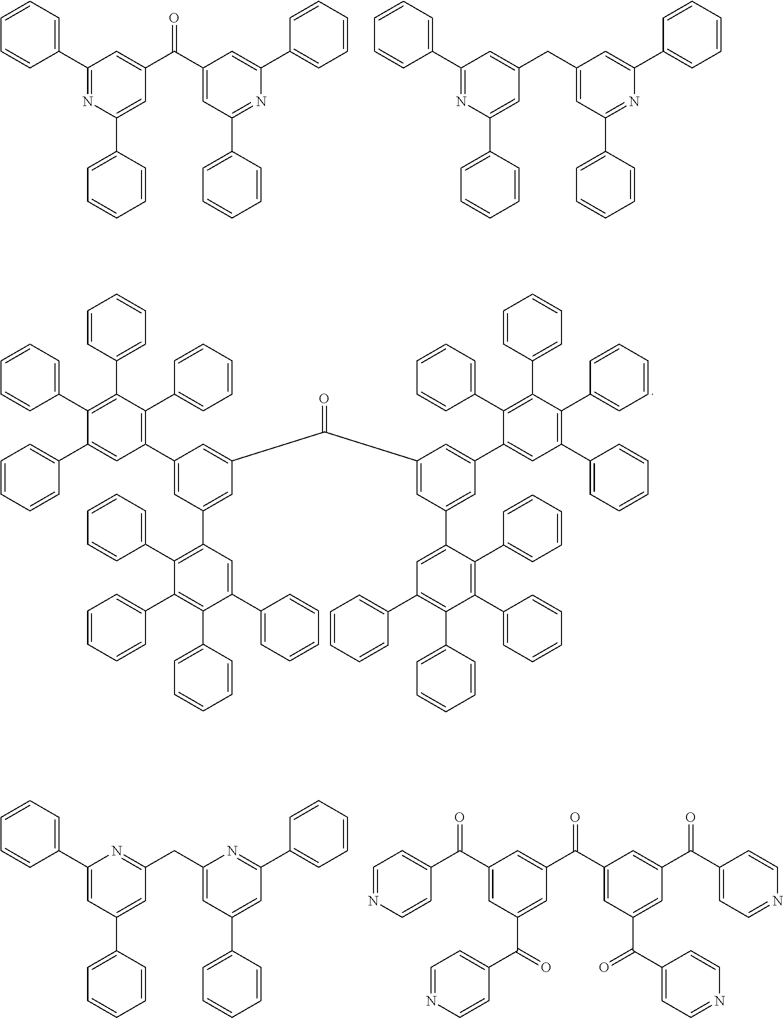

The compounds of the formula (1) preferably have a glass-transition temperature T_{G} of greater than 70° C., particularly preferably greater than 90° C.

For the purposes of this invention, an aryl group contains at least 6 C atoms; for the purposes of this invention, a heteroaryl group contains at least 2 C atoms and at least one heteroatom, with the proviso that the sum of C atoms and heteroatoms is at least 5. The heteroatoms are preferably selected from N, O and/or S. An aryl group or heteroaryl group here is taken to mean either a single aromatic ring, i.e. benzene, or a single heteroaromatic ring, for example pyridine, pyrimidine, thiophene, etc., or a condensed aryl or heteroaryl group, for example naphthalene, anthracene, pyrene, quinoline, isoquinoline, etc.

For the purposes of this invention, an aromatic ring system contains at least 6 C atoms in the ring system. For the purposes of this invention, a heteroaromatic ring system contains at least 2 C atoms and at least one heteroatom in the ring system, with the proviso that the sum of C atoms and heteroatoms is at least 5. The heteroatoms are preferably selected from N, O and/or S. For the purposes of this invention, an aromatic or heteroaromatic ring system is intended to be taken to mean a system which does not necessarily contain only aryl or heteroaryl groups, but instead in which a plurality of aryl or heteroaryl groups may also be interrupted by a short non-aromatic unit (preferably less than 10% of the atoms other than H), such as, for example, an sp^{a}-hybridised C, N or O atom. Thus, for example, systems such as 9,9′-spirobifluorene, 9,9-diarylfluorene, triarylamine, diaryl ether, stilbene, benzophenone, etc., are also intended to be taken to mean aromatic ring systems for the purposes of this invention. An aromatic or heteroaromatic ring system is likewise taken to mean systems in which a plurality of aryl or heteroaryl groups are linked to one another by single bonds, for example biphenyl, terphenyl or bipyridine.

For the purposes of the present invention, a C₁- to C₄₀-alkyl group, in which, in addition, individual H atoms or CH₂ groups may be substituted by the above-mentioned groups, is particularly preferably taken to mean the radicals methyl, ethyl, n-propyl, i-propyl, n-butyl, i-butyl, s-butyl, t-butyl, 2-methylbutyl, n-pentyl, s-pentyl, tert-pentyl, 2-pentyl, cyclopentyl, n-hexyl, s-hexyl, tert-hexyl, 2-hexyl, 3-hexyl, cyclohexyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl. A C₁- to C₄₀-alkoxy group is particularly preferably taken to mean methoxy, trifluoromethoxy, ethoxy, n-propoxy, i-propoxy, n-butoxy, i-butoxy, s-butoxy, tbutoxy or 2-methylbutoxy. An aromatic or heteroaromatic ring system having 5-60 aromatic ring atoms, which may also in each case be substituted by the above-mentioned radicals R and which may be linked to the aromatic or heteroaromatic ring via any desired positions, is taken to mean, in particular, groups derived from benzene, naphthalene, anthracene, phenanthrene, benzanthracene, pyrene, chrysene, perylene, fluoranthene, benzofluoranthene, naphthacene, pentacene, benzopyrene, biphenyl, biphenylene, terphenyl, terphenylene, fluorene, benzofluorene, dibenzofluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In a preferred embodiment of the invention, all symbols X in a cyclic system either stand for CR² or all symbols X in a cyclic system stand for N. Each of the two aromatic cyclic systems in formula (1) thus preferably stands either for a 3,5-substituted phenyl group or for a 4,6-substituted triazine group. Particularly preferably, all symbols X stand for CR².

In a further preferred embodiment of the invention, the symbol Y stands for C═O or C(R¹)₂, and R¹ stands, identically or differently on each occurrence, for H, F, a linear alkyl group having 1 to 10 C atoms, in particular having 1 to 6 C atoms, or a branched or cyclic alkyl group having 3 to 10 C atoms, in particular having 3 to 6 C atoms; two alkyl radicals R¹ here may form a ring system with one another. Particularly preferably, the symbol Y stands for C═O or C(R¹)₂, and R¹ stands for H, D, F or methyl. If one or more groups R, in particular all groups R, stand for an aromatic or heteroaromatic ring system, R¹ particularly preferably stands for C═O. If one or more groups R, in particular all groups R, stand for N(Ar)₂, Y particularly preferably stands for C(R¹)₂, in particular for CH₂ or C(CH₃)₂.

In a further preferred embodiment of the invention, the symbol R stands, identically or differently on each occurrence, for an aromatic or heteroaromatic ring system having 5 to 30 aromatic ring atoms, in particular an aromatic ring system having 6 to 30 aromatic ring atoms, which may in each case be substituted by one or more radicals R³, or for an N(Ar)₂, C(═O)Ar or P(═O)Ar₂ group. R very particularly preferably stands for an aromatic ring system having 6 to 30 aromatic ring atoms, which may be substituted by one or more radicals R³.

If the group R stands for an aromatic or heteroaromatic ring system, this is preferably selected from the groups phenyl, o-biphenyl, m-biphenyl, p-biphenyl, o-terphenyl, m-terphenyl, p-terphenyl, 3,5-(diphenyl)phenyl, m-quaterphenyl, 2-fluorenyl, 2-spirobifluorenyl, 1-naphthyl, 2-naphthyl, 1-, 2- or 9-anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, 2-, 4- or 5-pyrimidinyl, 1,3,5-triazinyl, in particular substituted by aromatic groups, N-benzimidazolyl, phenyl-N-benzimidazolyl, N-phenylbenzimidazolyl, phenyl-N-phenylbenzimidazolyl, thiophene, oxazole, oxadiazole, thiadiazole or benzothiazole. These groups may each be substituted by one or more substituents R³. Particularly preferred aromatic or heteroaromatic ring systems R are selected from structures of the following formulae (2) to (11), where the dashed bond in each case indicates the linking of this unit and where these groups may each be substituted by one or more radicals R³:

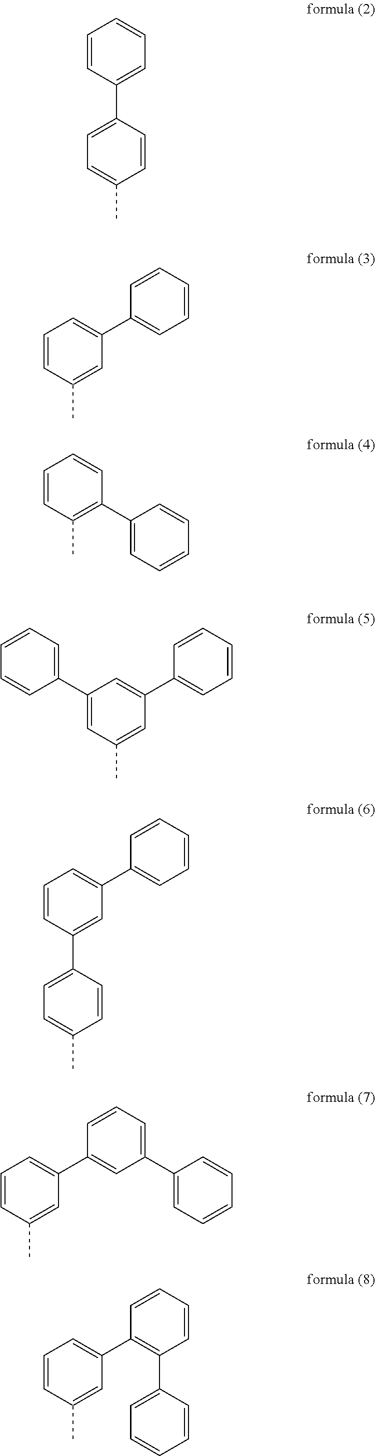

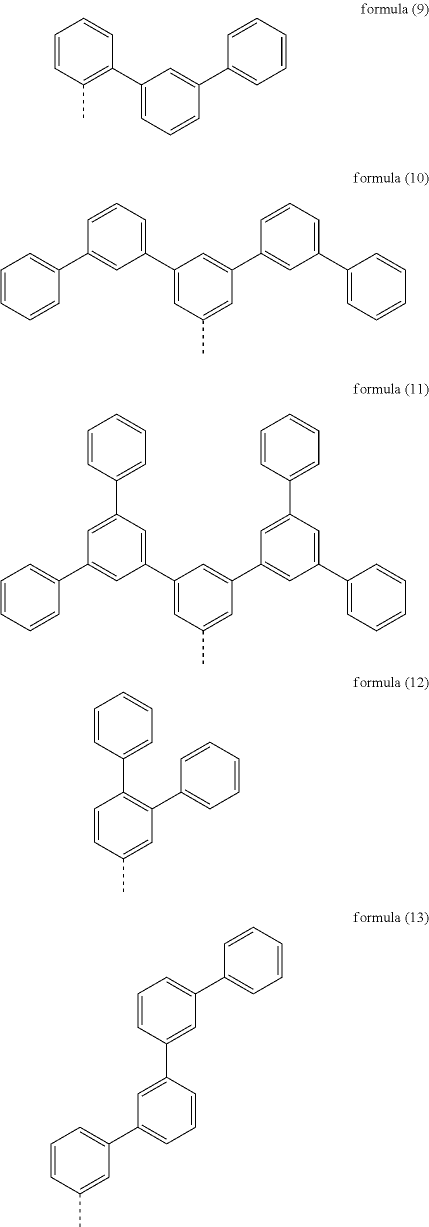

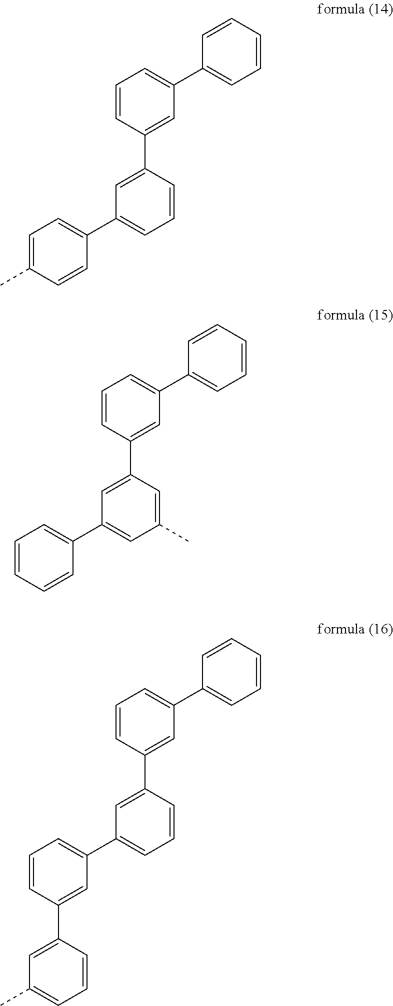

Of the structures of the formulae (2) to (16), the structures of the formulae (3), (5), (7), (10), (11), (13) and (15) are very particularly preferred.

If the radical R stands for an N(Ar)₂ group, this group is preferably selected from the groups of the formula (17) or formula (18):

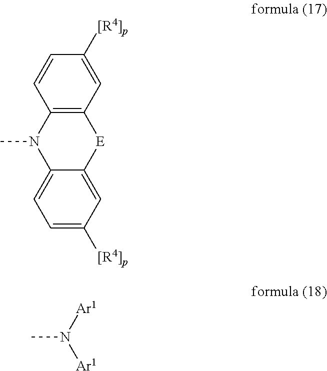

where R⁴ has the meaning indicated above, and furthermore:
E stands for a single bond, O, S, N(R⁴) or C(R⁴)₂;
Ar¹ is, identically or differently on each occurrence, an aromatic or heteroaromatic ring system having 5 to 24 aromatic ring atoms or a triarylamine group having 15 to 30 aromatic ring atoms, each of which may be substituted by one or more radicals R⁴, preferably an aryl or heteroaryl group having 6 to 14 aromatic ring atoms or a triarylamine group having 18 to 30 aromatic ring atoms, preferably having 18 to 22 aromatic ring atoms, each of which may be substituted by one or more radicals R⁴;
p is on each occurrence, identically or differently, 0 or 1.
Ar¹ particularly preferably stands, identically or differently on each occurrence, for phenyl, biphenyl, 1-naphthyl, 2-naphthyl, 2-spirobifluorenyl, 2-, 3- or 4-triphenylamine, 1- or 2-naphthyldiphenylamine, each of which may be bonded via the naphthyl or phenyl group, 1- or 2-dinaphthylphenylamine, each of which may be bonded via the naphthyl or phenyl group, N-carbazolyl, N-phenyl-2-carbazolyl or N-phenyl-3-carbazolyl. These groups may each be substituted by one or more alkyl groups having 1 to 4 C atoms or by fluorine.

In a preferred embodiment of the invention, all symbols R in compounds of the formula (1) are selected identically. In a further preferred embodiment of the invention, both substituents R which are bonded to the same ring are each selected identically, but differ from the substituents R on the other ring.

In a further preferred embodiment of the invention, the symbol R² stands, identically or differently on each occurrence, for H, F, a straight-chain alkyl group having 1 to 10 C atoms, in particular having 1 to 6 C atoms, or a branched or cyclic alkyl group having 3 to 10 C atoms, in particular having 3 to 6 C atoms. R² particularly preferably stands for H, F or methyl, in particular for H.

The compounds of the formula (1) particularly preferably have the preferences mentioned above at the same time. Particularly preferred embodiments of the compounds of the formula (1) are therefore the compounds of the formulae (19), (20) and (21):

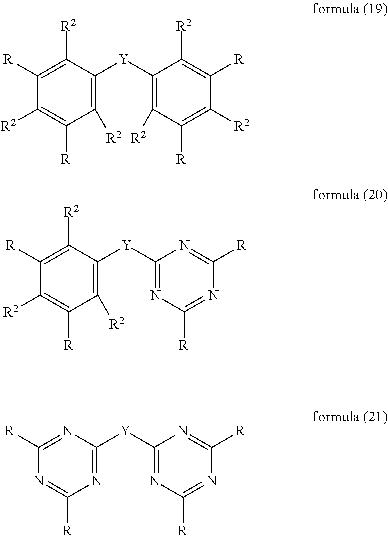

where Ar, R³ and R⁴ are as defined above, and the following applies to the other symbols used:
Y is C═O or C(R¹)₂;
R is on each occurrence, identically or differently, an aromatic or heteroaromatic ring system having 5 to 30 aromatic ring atoms, preferably an aromatic ring system having 6 to 30 aromatic ring atoms, in particular selected from the group consisting of phenyl, o-biphenyl, m-biphenyl, p-biphenyl, o-terphenyl, m-terphenyl, p-terphenyl, 3,5-(diphenyl)phenyl, m-quaterphenyl, 1-naphthyl, 2-naphthyl, anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, N-benzimidazolyl, phenyl-N-benzimidazolyl, N-phenylbenzimidazolyl or phenyl-N-phenylbenzimidazolyl, in particular selected from formulae (2) to (16) depicted above or an N(Ar)₂ group, preferably selected from formulae (17) and (18) depicted above, C(═O)Ar or P(═O)Ar₂;
R¹ is on each occurrence, identically or differently, H, F, a linear alkyl group having 1 to 10 C atoms, preferably having 1 to 6 C atoms, in particular methyl, or a branched or cyclic alkyl group having 3 to 10 C atoms, preferably having 3 to 6 C atoms; a plurality of radicals R¹ here may form a ring system with one another;
R² is on each occurrence, identically or differently, H, F, a straight-chain alkyl group having 1 to 10 C atoms, in particular having 1 to 6 C atoms, or a branched or cyclic alkyl group having 3 to 10 C atoms, in particular having 3 to 6 C atoms, preferably H, F or methyl, particularly preferably H.

A particularly preferred embodiment of the compounds of the formula (1) are the compounds of the formula (22)

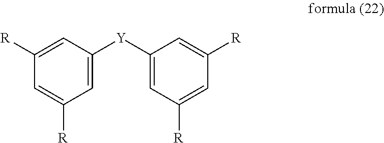

where Ar, R³ and R⁴ are as defined above, and the following applies to the other symbols used:
Y is C═O, CH₂, CF₂ or C(alkyl)₂, where alkyl represents an alkyl group having 1 to 6 C atoms, in particular methyl;
R is on each occurrence, identically or differently, an aromatic ring system having 6 to 30 aromatic ring atoms, in particular selected from the group consisting of phenyl, o-biphenyl, m-biphenyl, p-biphenyl, o-terphenyl, m-terphenyl, p-terphenyl, 3,5-(diphenyl)phenyl, m-quaterphenyl, 1-naphthyl, 2-naphthyl, anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, N-benzimidazolyl, phenyl-N-benzimidazolyl, N-phenylbenzimidazolyl or phenyl-N-phenylbenzimidazolyl, in particular groups of the formulae (2) to (16) depicted above or an N(Ar)₂ group, preferably selected from formulae (17) and (18) depicted above, or C(═O)Ar or P(═O)Ar₂.

In a further preferred embodiment of the invention, the symbol Ar in cornpounds of the formula (1) and formulae (19) to (22) stands, identically or differently on each occurrence, for an aromatic ring system having 6 to 30 aromatic ring atoms, in particular selected from the group consisting of phenyl, o-biphenyl, m-biphenyl, p-biphenyl, o-terphenyl, m-terphenyl, p-terphenyl, 3,5-(diphenyl)phenyl, m-quaterphenyl, 1-naphthyl, 2-naphthyl, anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, N-benzimidazolyl, phenyl-N-benzimidazolyl, N-phenylbenzimidazolyl or phenyl-N-phenylbenzimidazolyl.

In a further preferred embodiment of the invention, the symbol R³ in compounds of the formula (1) and formulae (19) to (22) stands, identically or differently on each occurrence, for H, F, N(Ar)₂, C(═O)Ar, P(═O)(Ar)₂, S(═O)Ar, S(═O)₂Ar, CR²═CR²Ar, Si(R⁴)₃, B(OR⁴)₂, B(N(R⁴)₂)₂, a straight-chain alkyl or alkoxy group having 1 to 10 C atoms or a branched or cyclic alkyl or alkoxy group having 3 to 10 C atoms, each of which may be substituted by one or more radicals R⁴, where one or more non-adjacent CH₂ groups may be replaced by R⁴C═CR⁴ or C═O, NR⁴, O or S and where one or more H atoms may be replaced by F, or an aromatic or heteroaromatic ring system having 5 to 30 aromatic ring atoms, which may in each case be substituted by one or more radicals R⁴, or an aryloxy or heteroaryloxy group having 5 to 30 aromatic ring atoms, which may be substituted by one or more radicals R⁴, or a combination of these systems; two or more adjacent substituents R³ here may also form a mono- or polycyclic, aliphatic or aromatic ring system with one another. R³ particularly preferably stands, identically or differently on each occurrence, for H, F, N(Ar)₂, C(═O)Ar, P(═O)(Ar)₂, Si(R⁴)₃, B(OR⁴)₂, B(N(R⁴)₂)₂, a straight-chain alkyl group having 1 to 6 C atoms, in particular methyl or ethyl, or a branched or cyclic alkyl group having 3 to 6 C atoms, each of which may be substituted by one or more radicals R⁴, where one or more H atoms may be replaced by F, in particular isopropyl or tert-butyl, or an aromatic or heteroaromatic ring system having 5 to 20 aromatic ring atoms, which may in each case be substituted by one or more radicals R⁴, or a combination of these systems; two or more adjacent substituents R³ here may also form a mono- or polycyclic, aliphatic or aromatic ring system with one another. In the case of compounds which are processed from solution, linear or branched alkyl groups having up to 10 C atoms are also preferred.

Examples of preferred compounds of the formulae (1) and (19) to (22) are structures (1) to (250) depicted below.

| | |
|---|---|
| (1) | 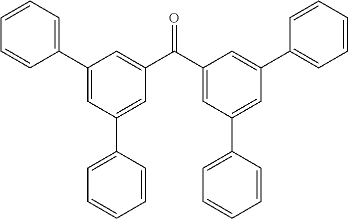 |
| (2) | 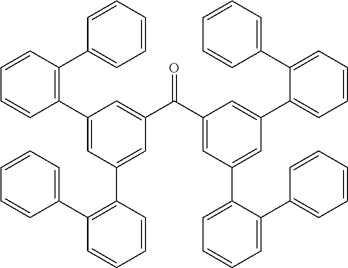 |
| (3) | 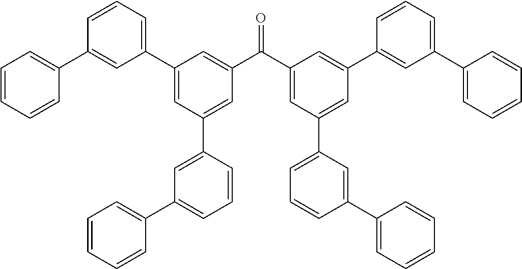 |
| (4) | 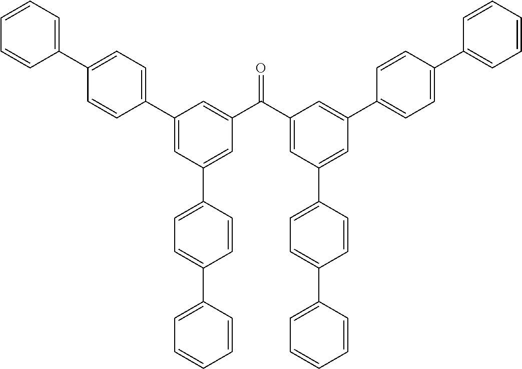 |
| (5) | 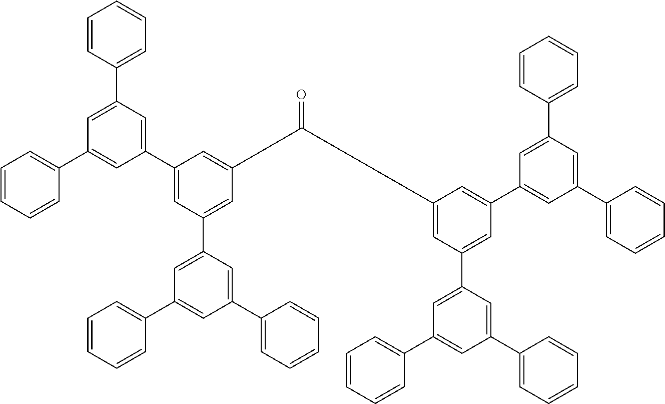 |
| (6) | 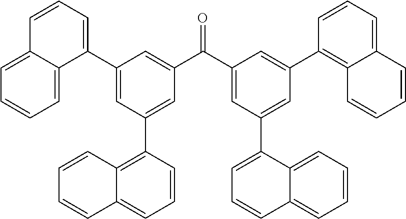 |
| (7) | 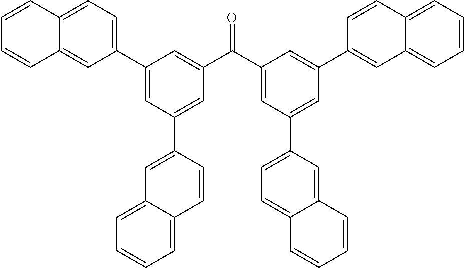 |
| (8) | 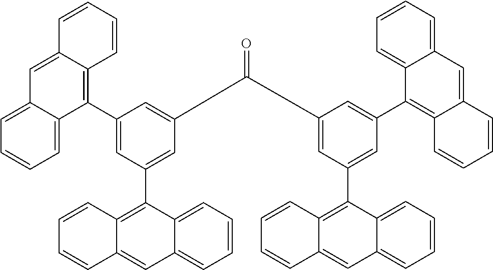 |
| (9) | 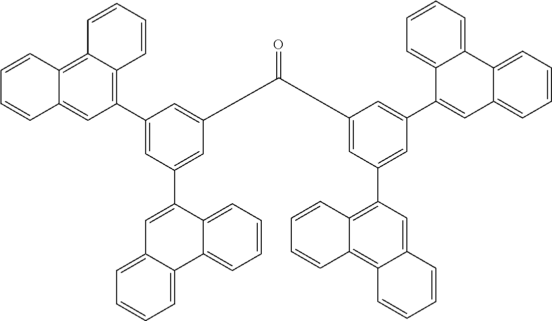 |
| (10) | 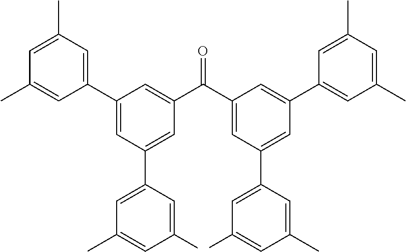 |
| (11) | 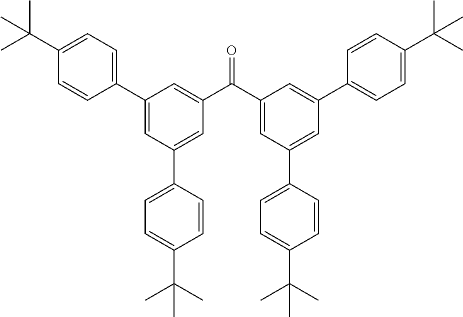 |
| (12) | 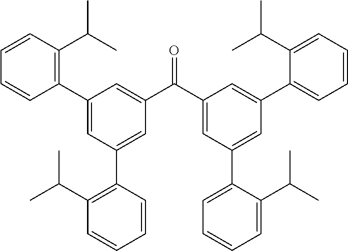 |
| (13) | 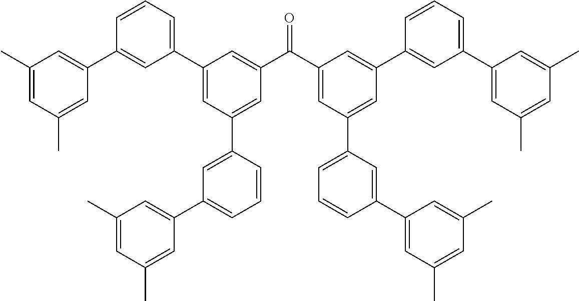 |
| (14) | 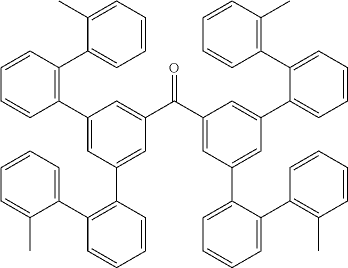 |
| (15) | 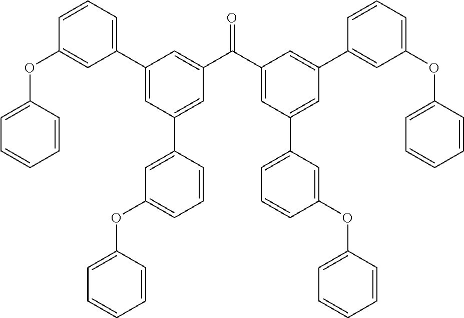 |
| (16) | 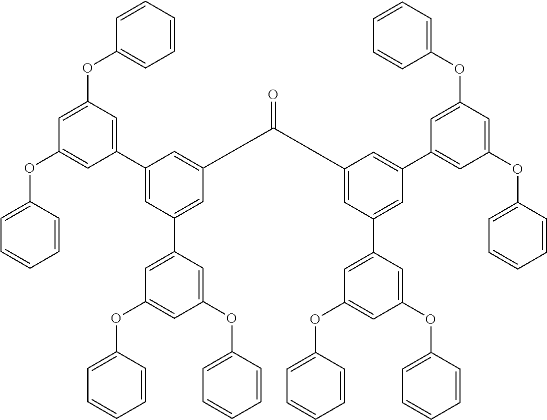 |
| (17) | 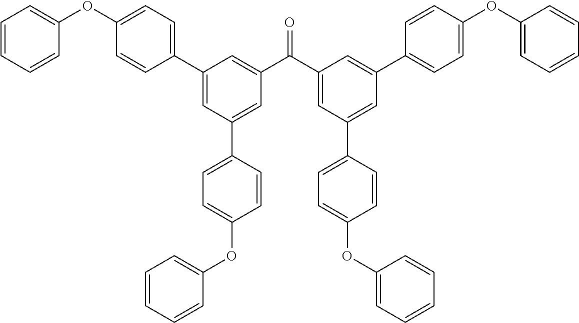 |
| (18) | 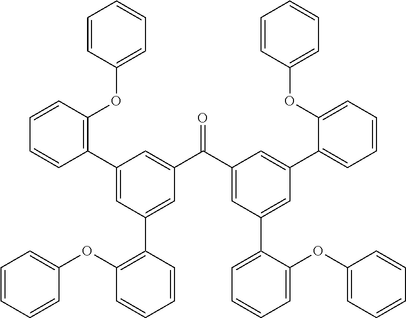 |
| (19) | 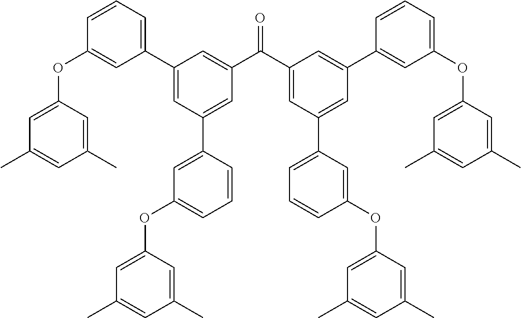 |
| (20) | 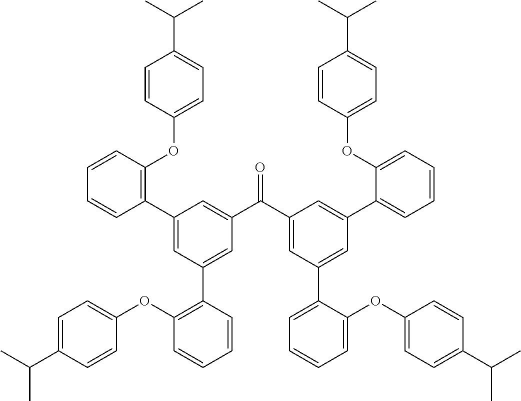 |
| (21) | 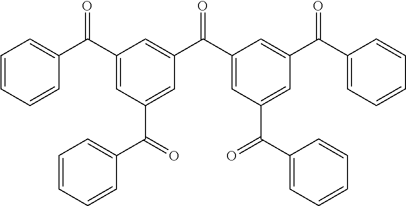 |
| (22) | 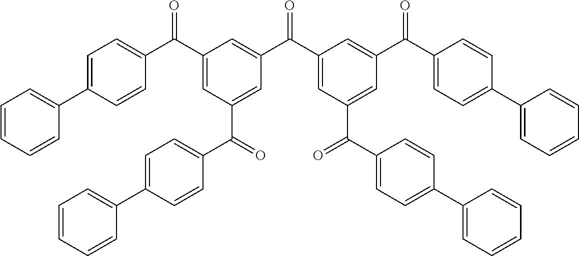 |
| (23) | 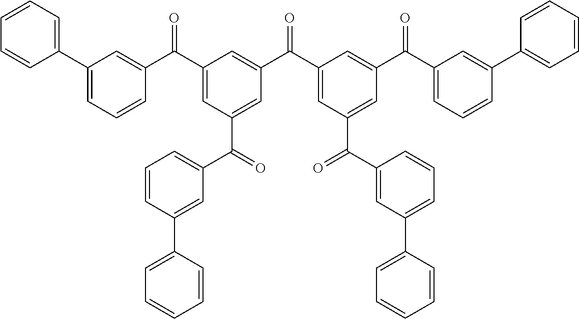 |
| (24) | 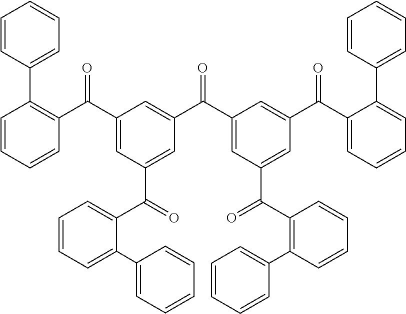 |
| (25) | 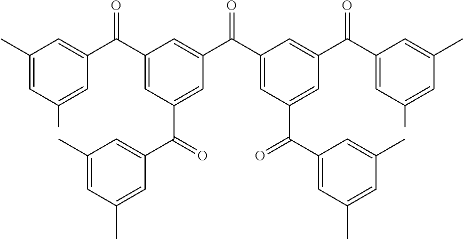 |
| (26) | 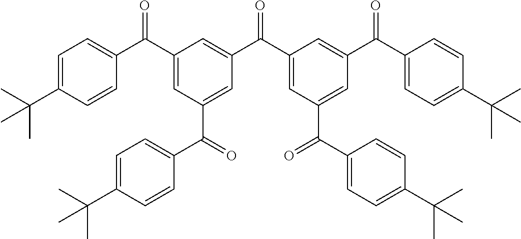 |
| (27) | 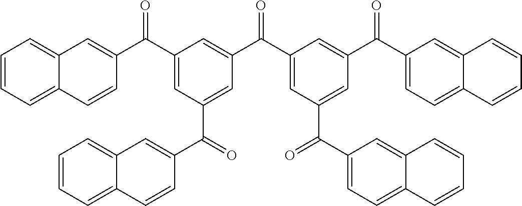 |
| (28) | 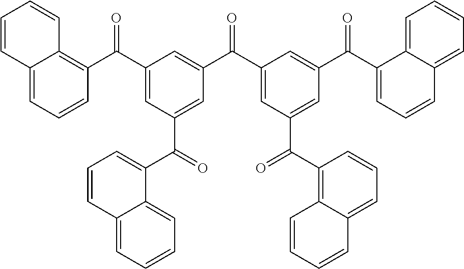 |
| (29) | 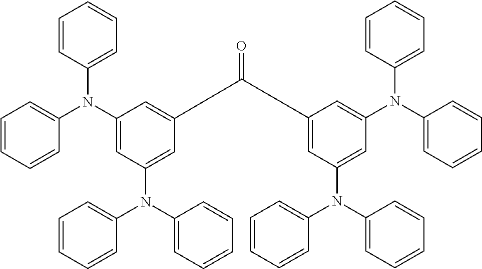 |
| (30) | 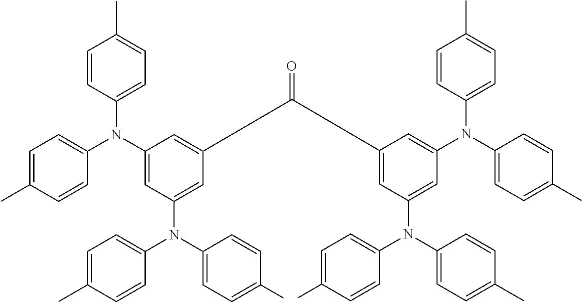 |
| (31) | 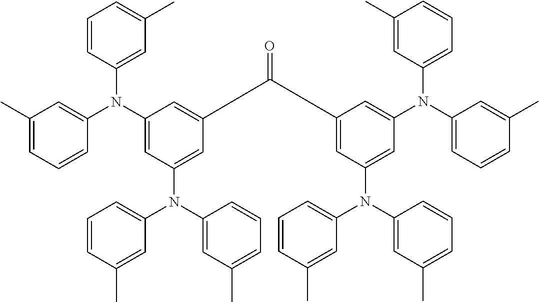 |
| (32) | 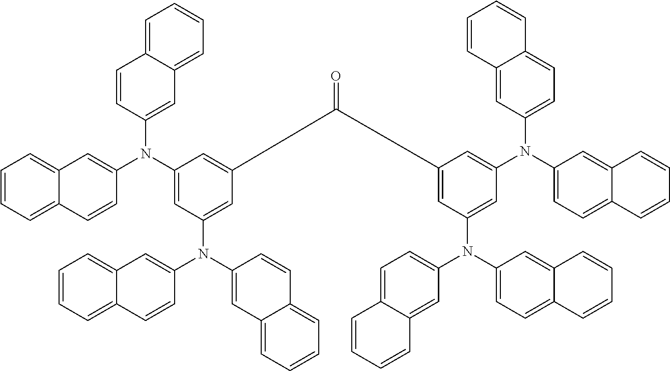 |
| (33) | 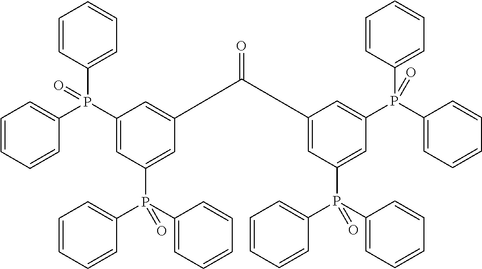 |
| (34) | 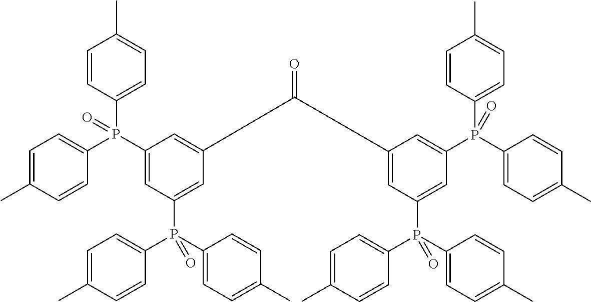 |
| (35) | 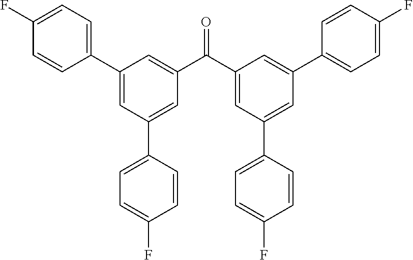 |
| (36) | 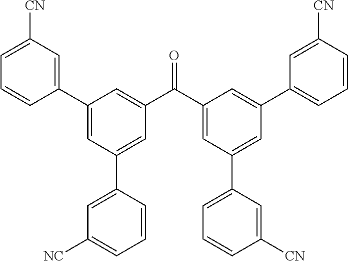 |
| (37) | 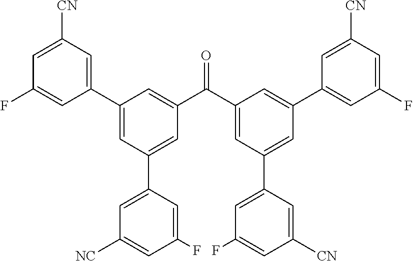 |
| (38) | 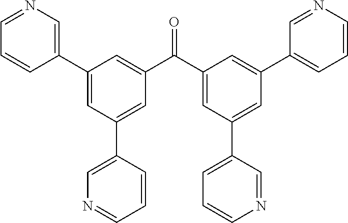 |
| (39) | 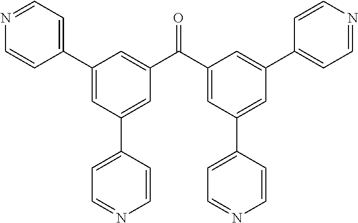 |
| (40) | 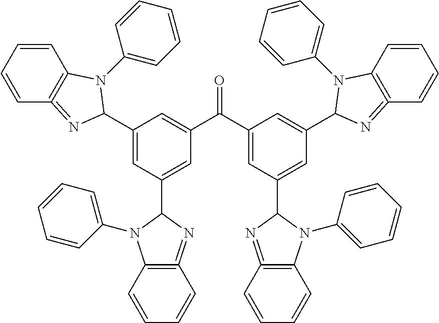 |
| (41) | 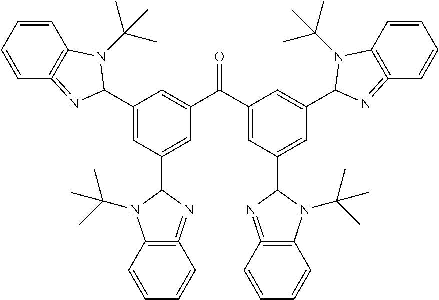 |
| (42) | 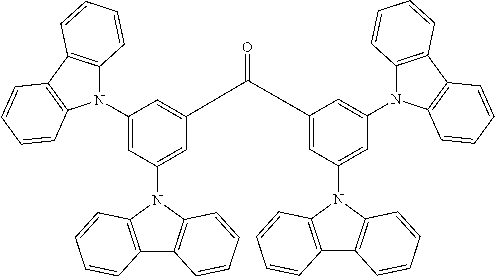 |
| (43) | 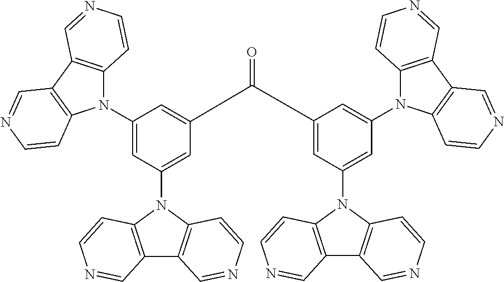 |
| (44) | 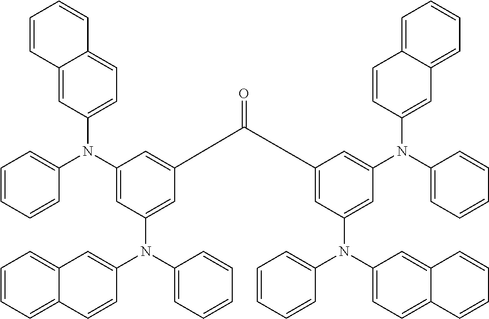 |
| (45) | 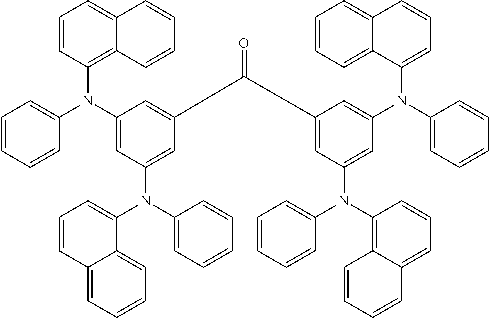 |
| (46) | 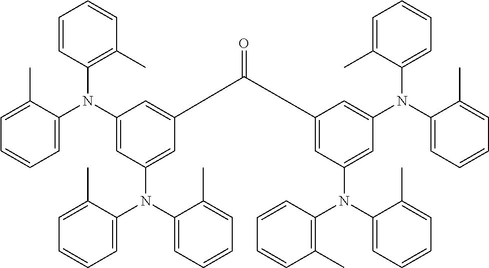 |
| (47) | 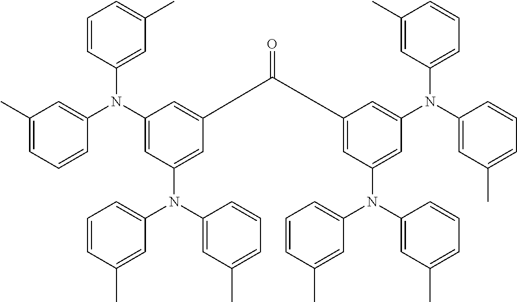 |
| (48) | 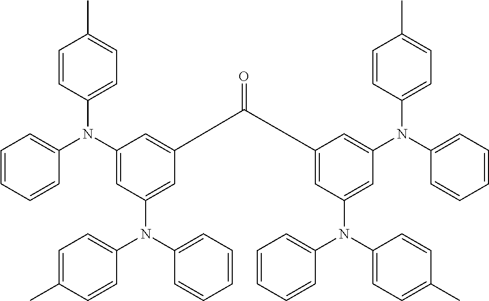 |
| (49) | 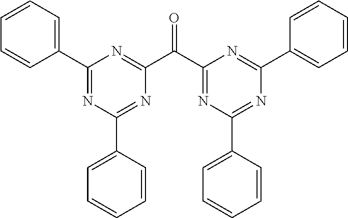 |
| (50) | 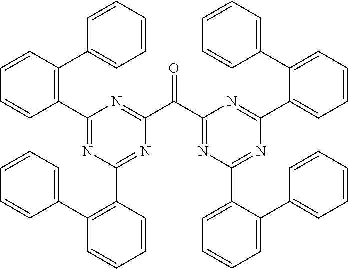 |
| (51) | 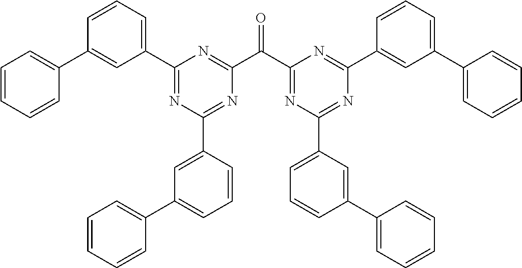 |
| (52) | 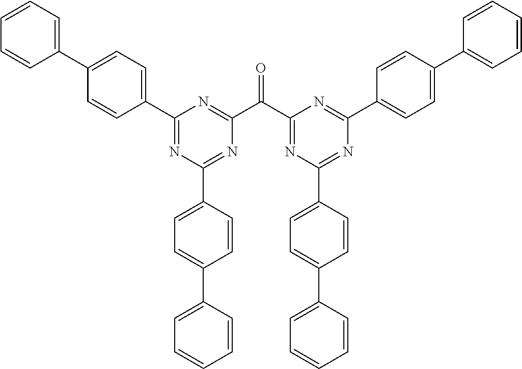 |
| (53) | 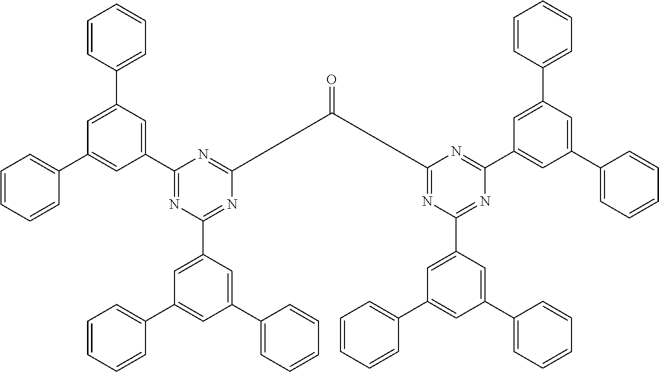 |
| (54) | 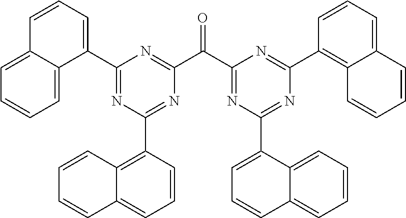 |
| (55) | 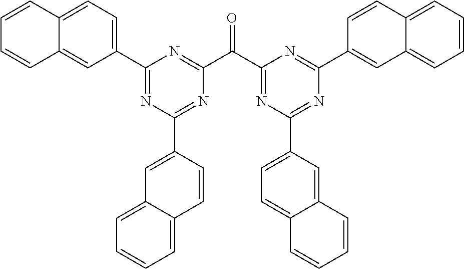 |
| (56) | 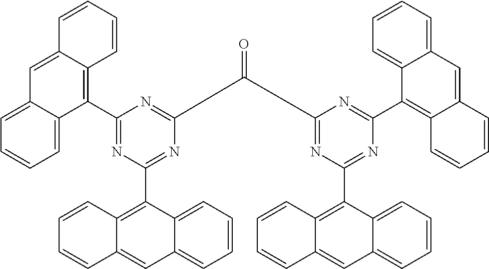 |
| (57) | 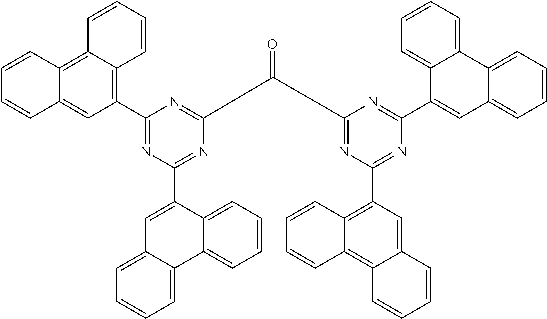 |
| (58) | 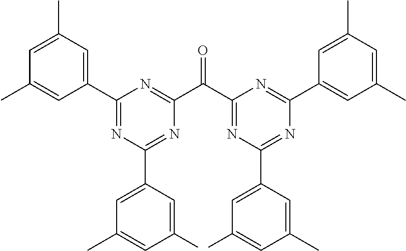 |
| (59) | 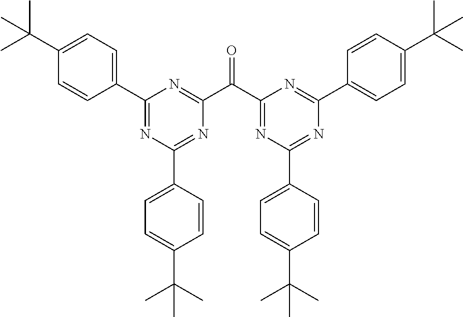 |
| (60) | 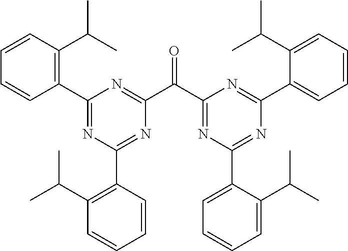 |
| (61) | 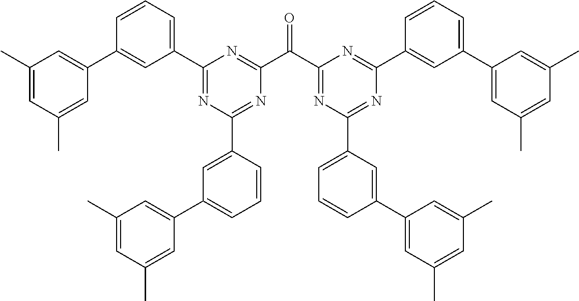 |
| (62) | 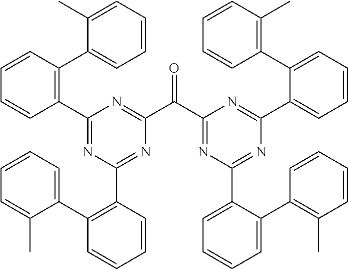 |
| (63) | 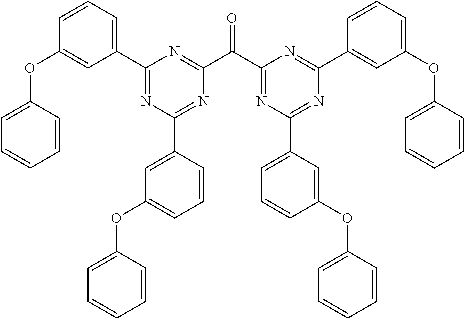 |
| (64) | 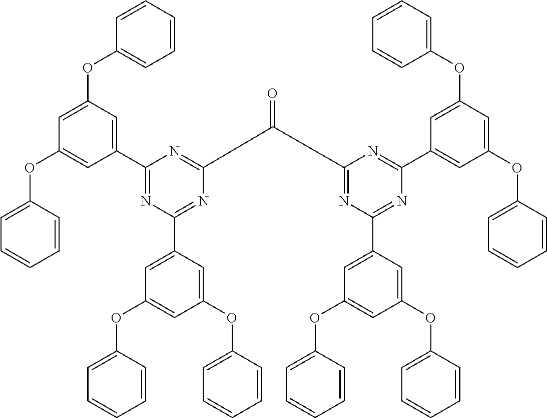 |
| (65) | 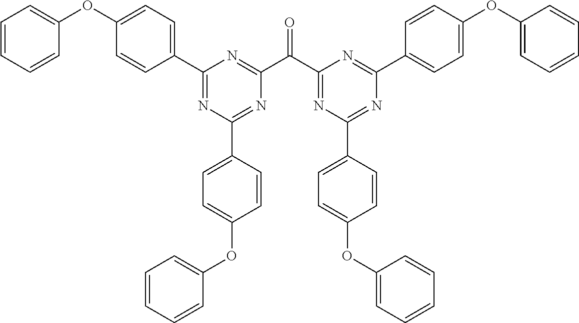 |
| (66) | 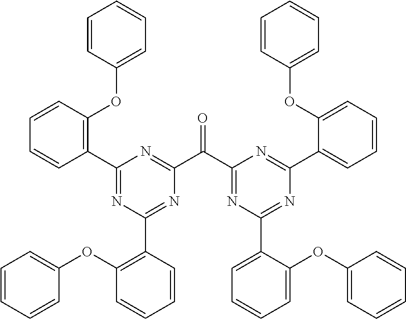 |
| (67) | 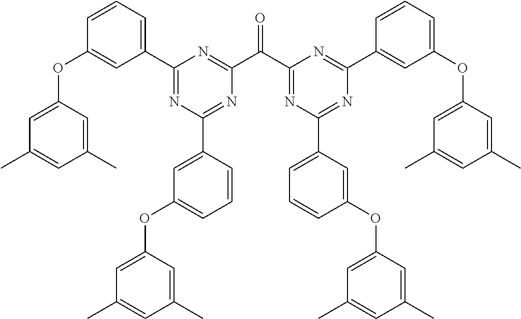 |
| (68) | 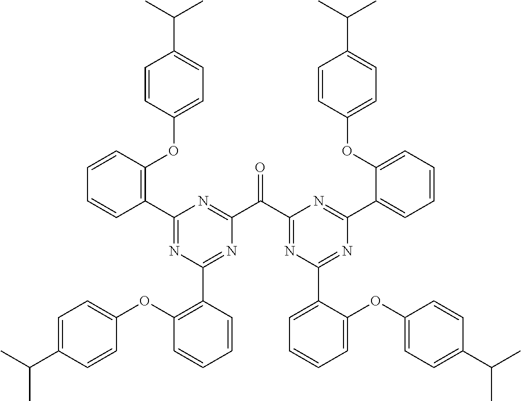 |
| (69) | 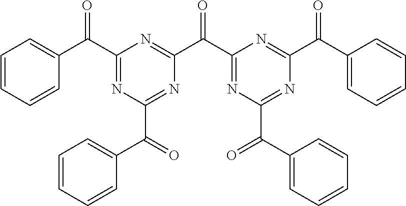 |
| (70) | 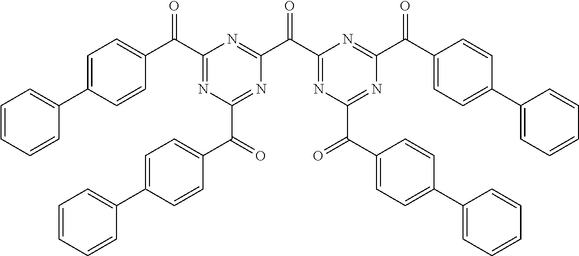 |
| (71) | 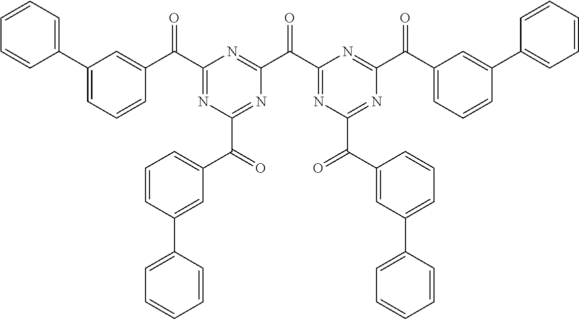 |
| (72) | 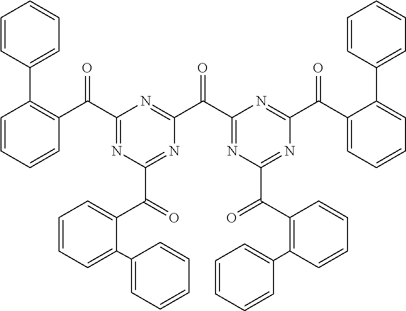 |
| (73) | 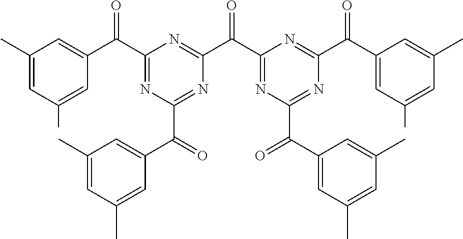 |
| (74) | 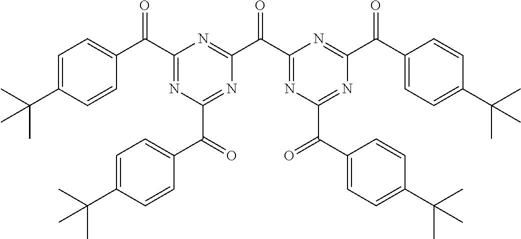 |
| (75) | 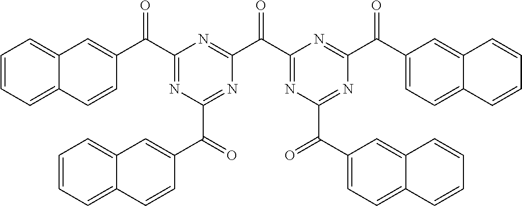 |
| (76) | 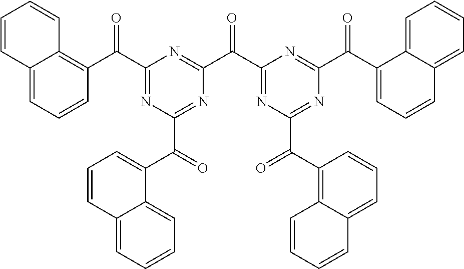 |
| (77) | 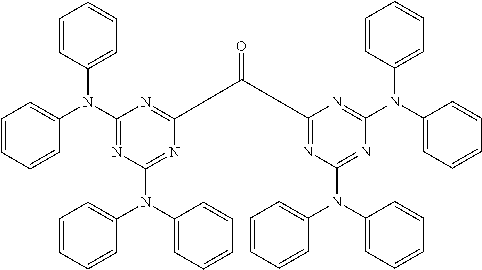 |
| (78) | 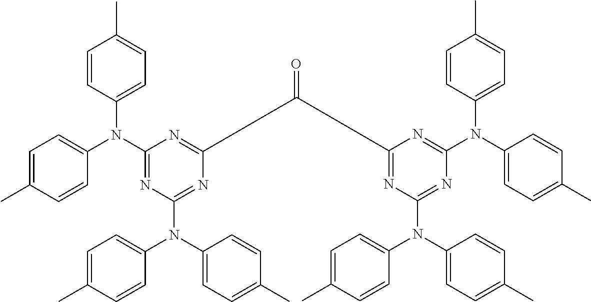 |
| (79) | 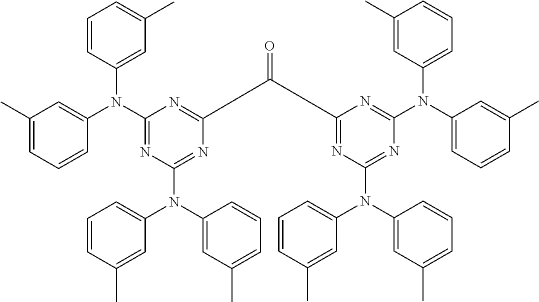 |
| (80) | 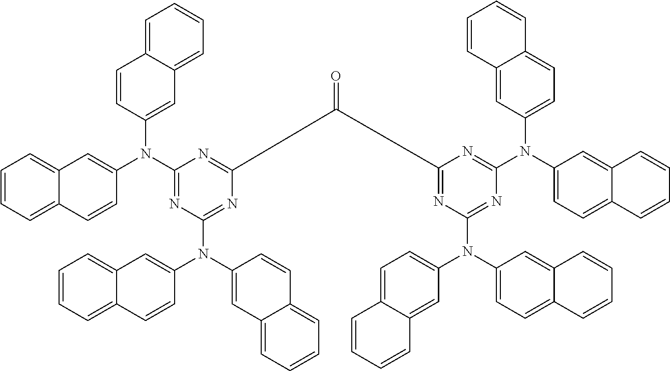 |
| (81) | 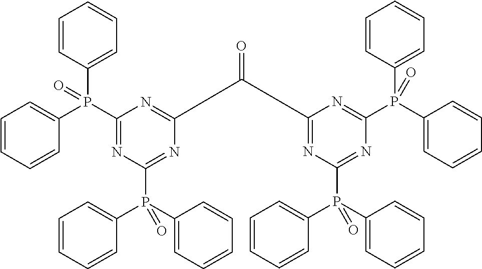 |
| (82) | 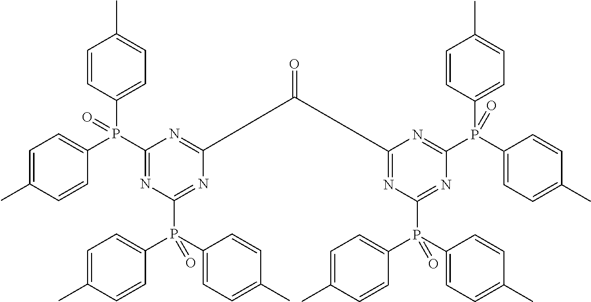 |
| (83) | 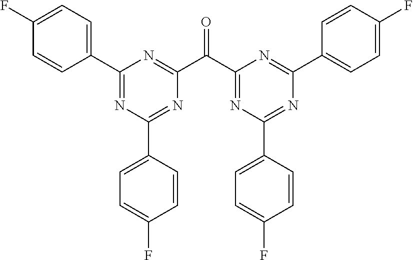 |
| (84) | 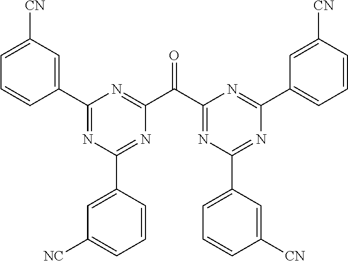 |
| (85) | 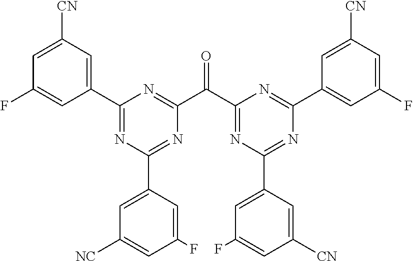 |
| (86) | 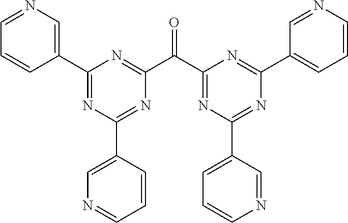 |
| (87) | 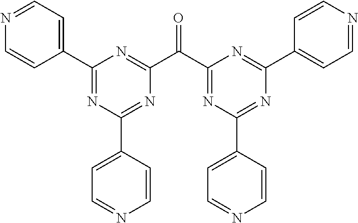 |
| (88) | 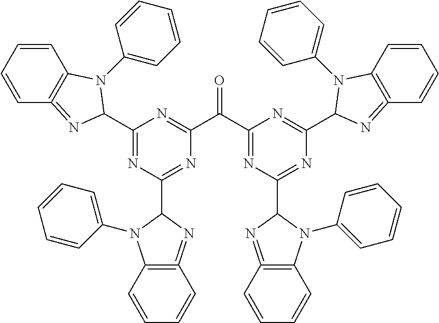 |
| (89) | 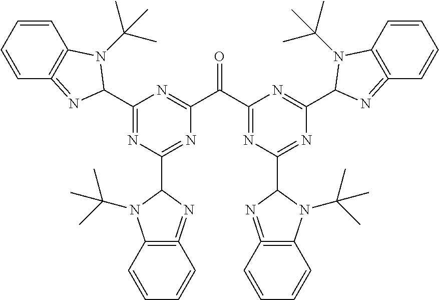 |
| (90) | 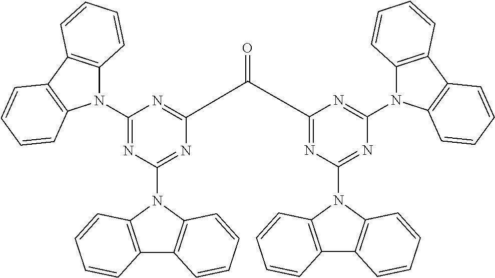 |
| (91) | 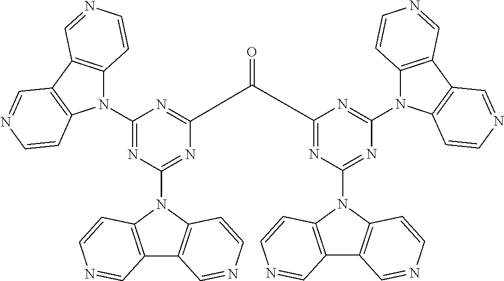 |
| (92) | 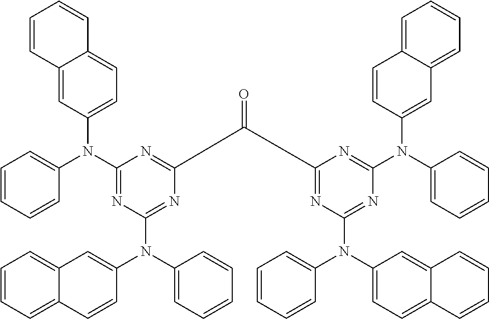 |
| (93) | 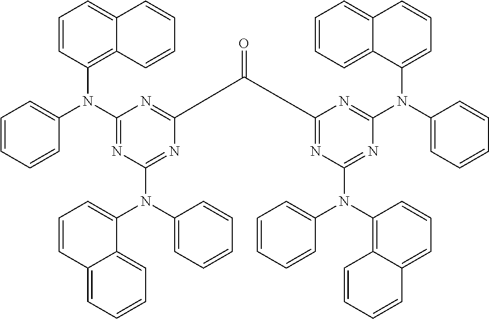 |
| (94) | 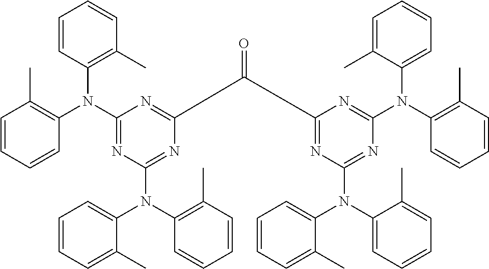 |
| (95) | 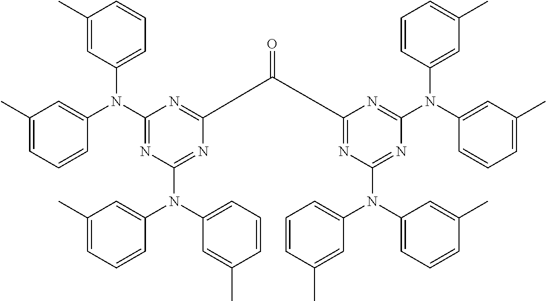 |
| (96) | 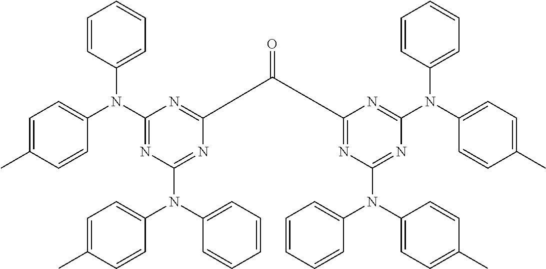 |
| (97) | 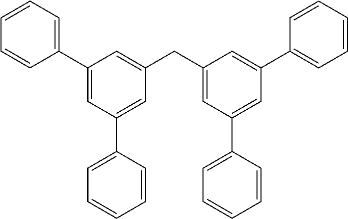 |
| (98) | 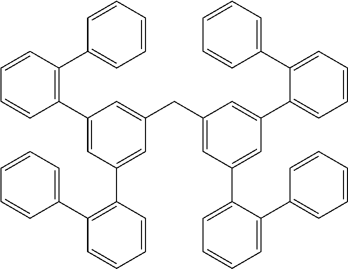 |
| (99) | 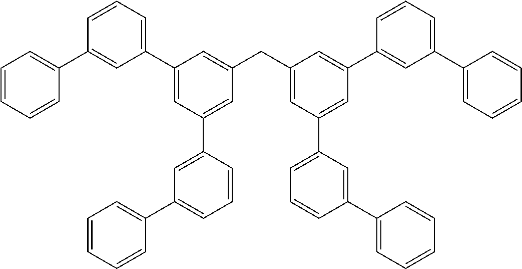 |
| (100) | 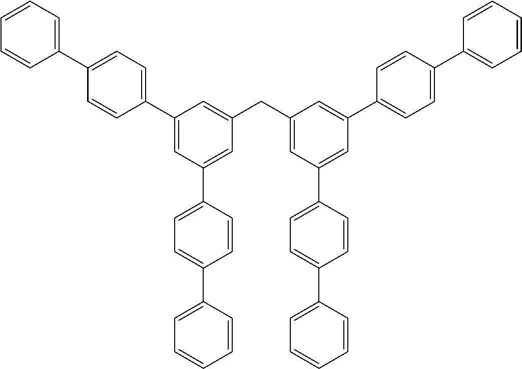 |
| (101) | 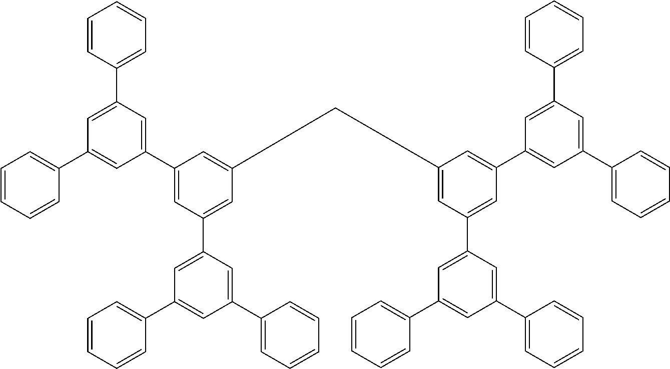 |
| (102) | 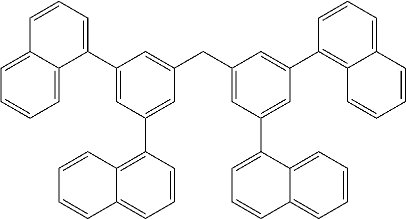 |
| (103) | 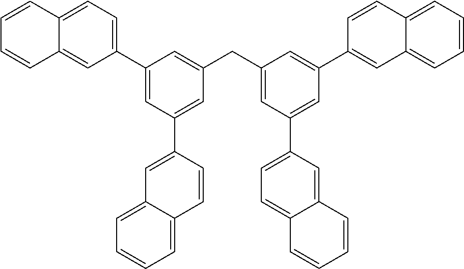 |
| (104) | 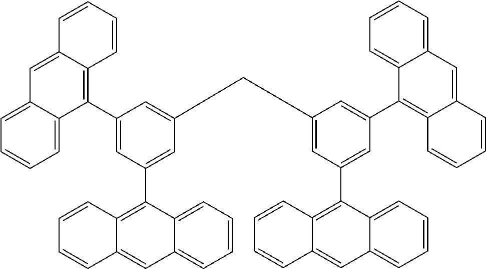 |
| (105) | 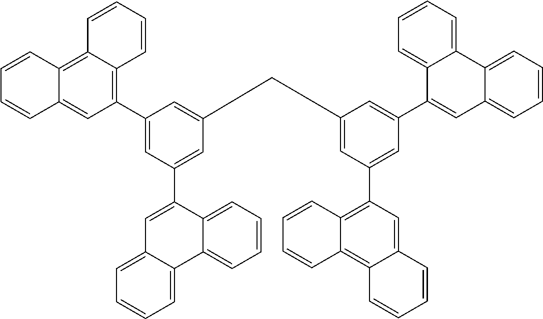 |
| (106) | 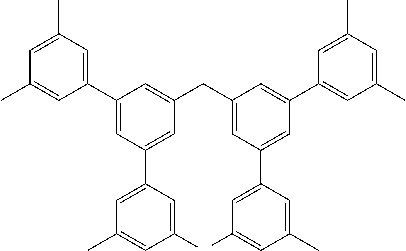 |
| (107) | 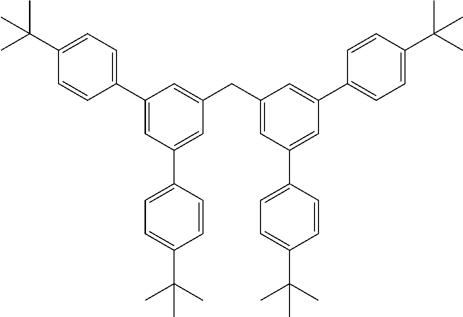 |
| (108) | 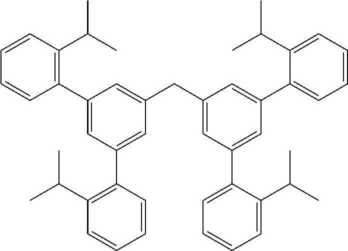 |
| (109) | 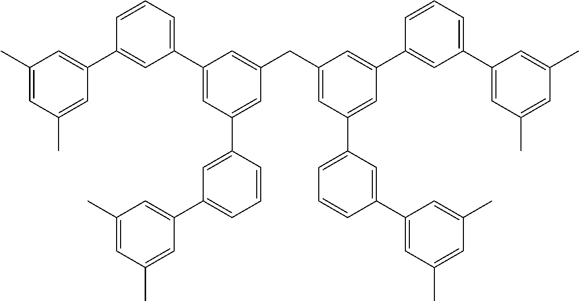 |
| (110) | 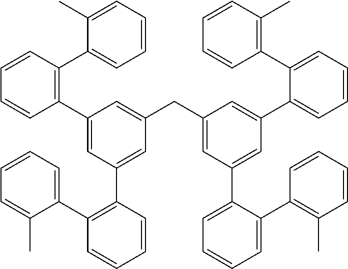 |
| (111) | 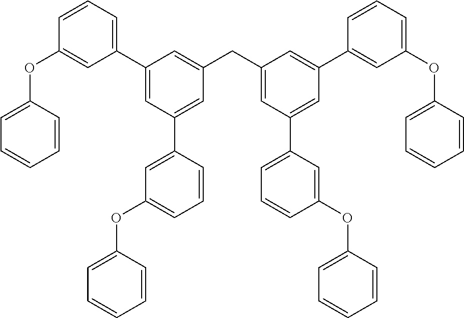 |
| (112) | 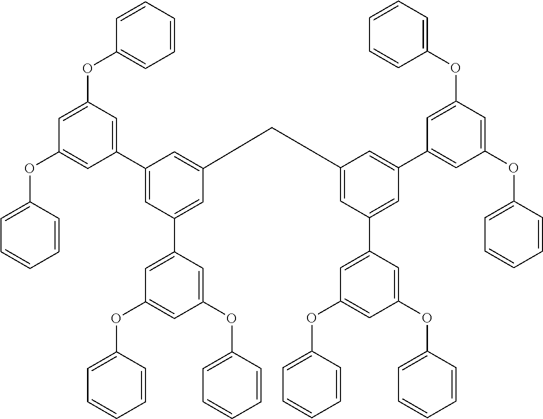 |
| (113) | 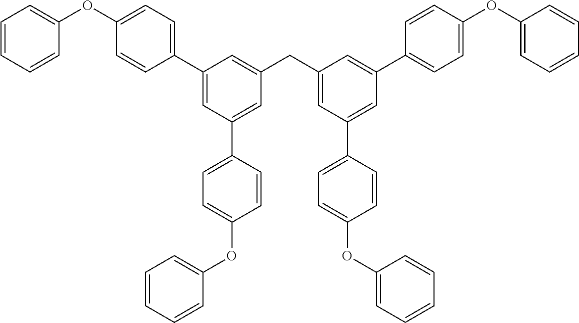 |
| (114) | 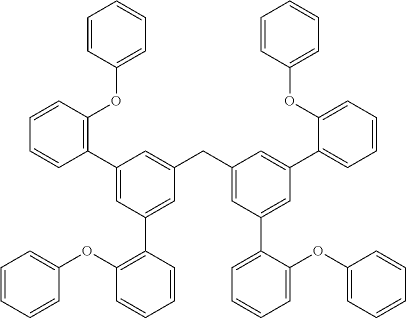 |
| (115) | 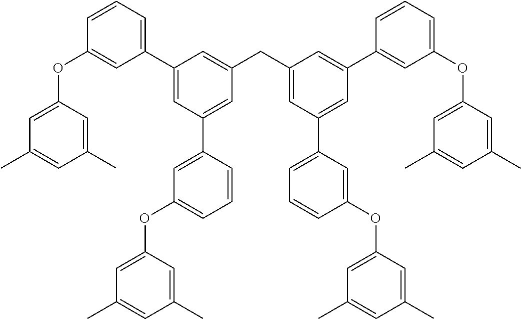 |
| (116) | 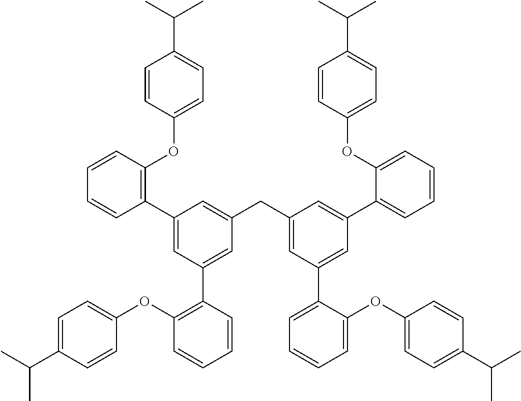 |
| (117) | 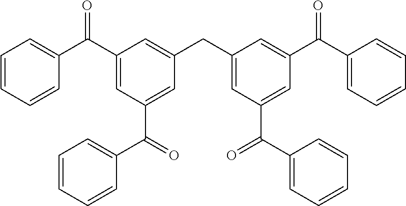 |
| (118) | 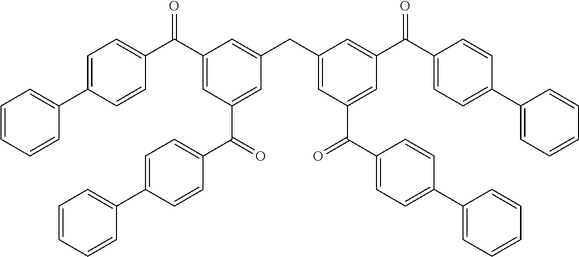 |
| (119) | 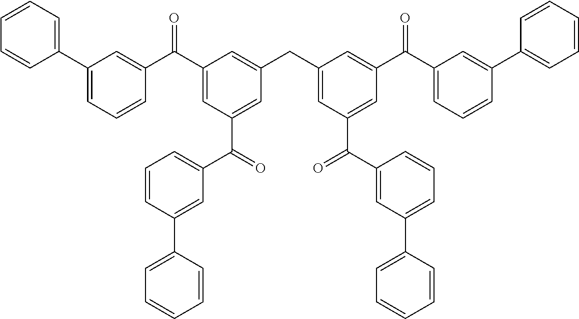 |
| (120) | 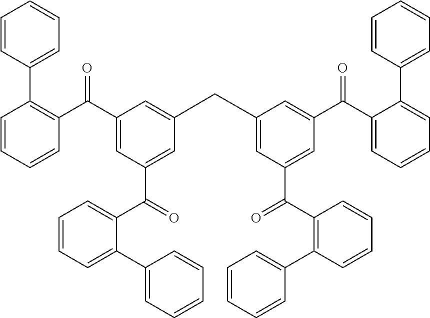 |
| (121) | 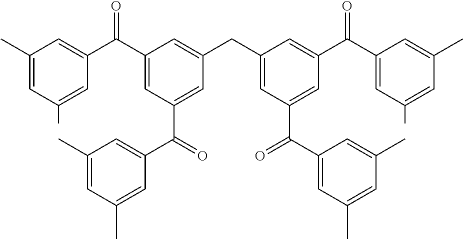 |
| (122) | 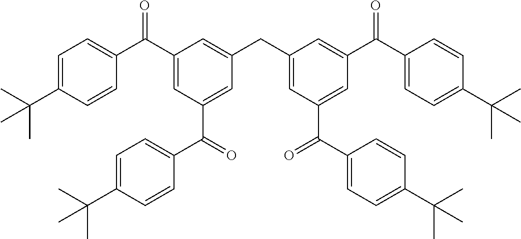 |
| (123) | 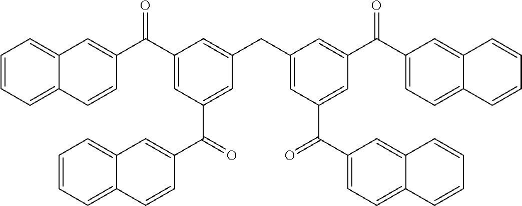 |
| (124) | 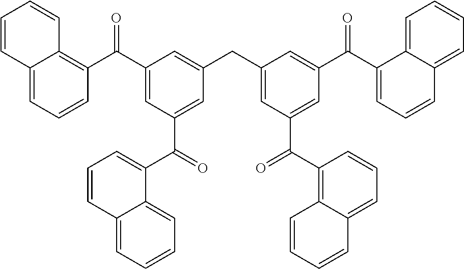 |
| (125) | 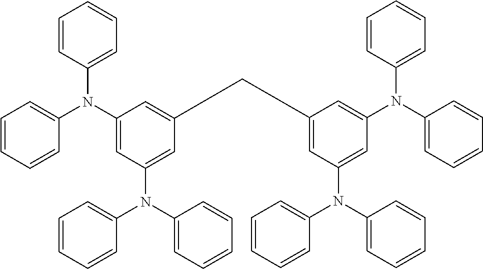 |
| (126) | 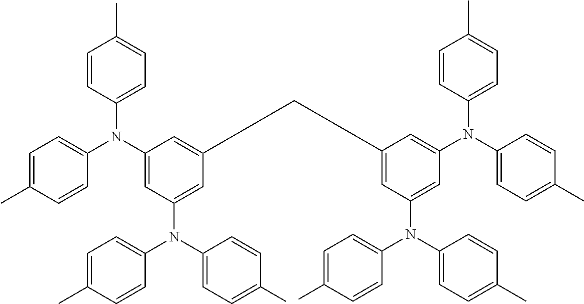 |
| (127) | 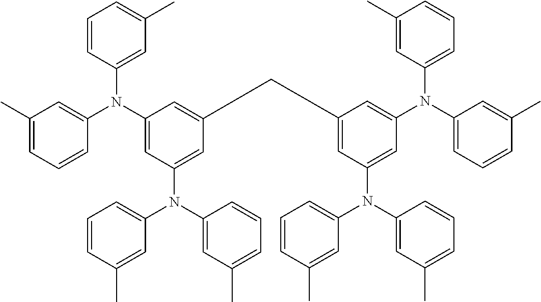 |
| (128) | 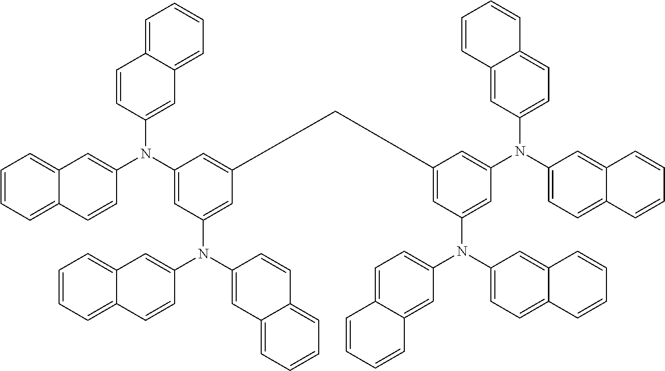 |
| (129) | 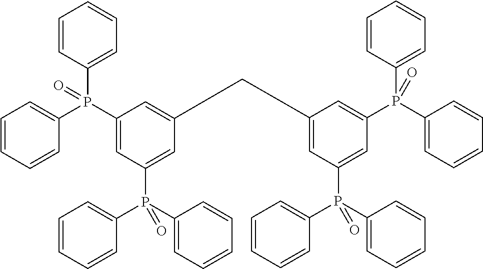 |
| (130) | 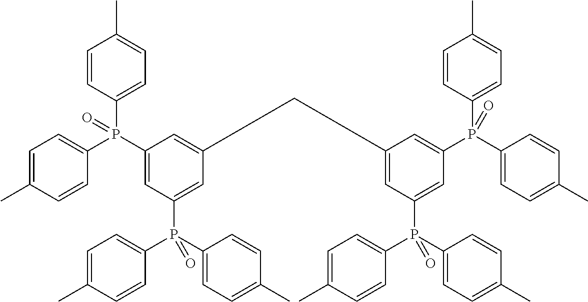 |
| (131) | 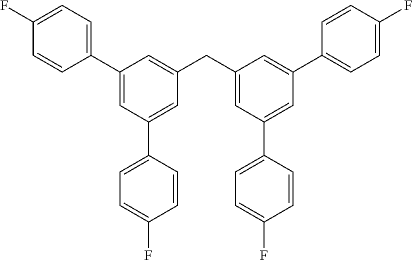 |
| (132) | 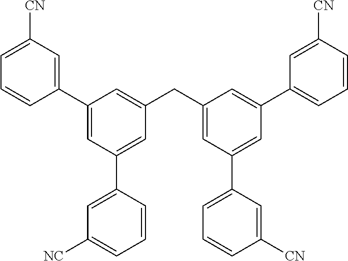 |
| (133) | 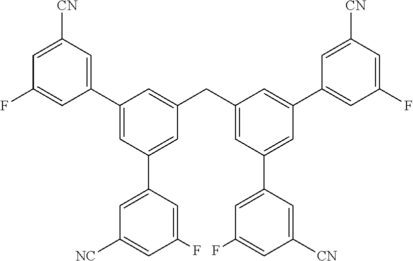 |
| (134) | 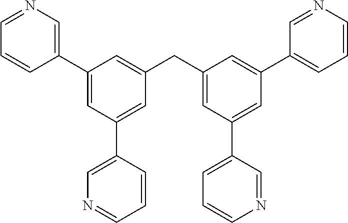 |
| (135) | 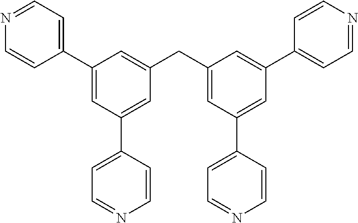 |
| (136) | 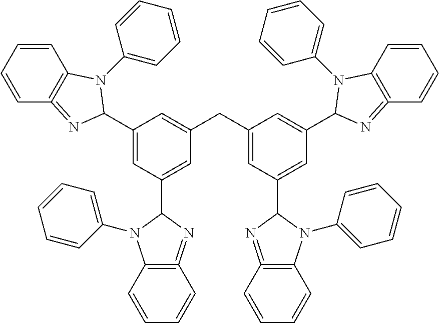 |
| (137) | 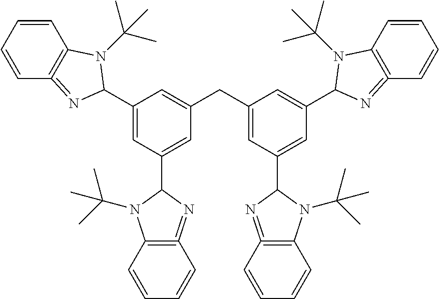 |
| (138) | 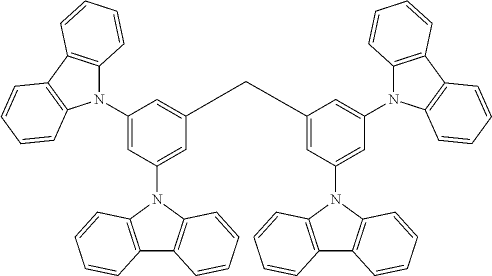 |
| (139) | 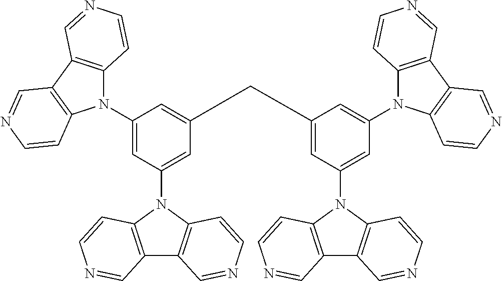 |
| (140) | 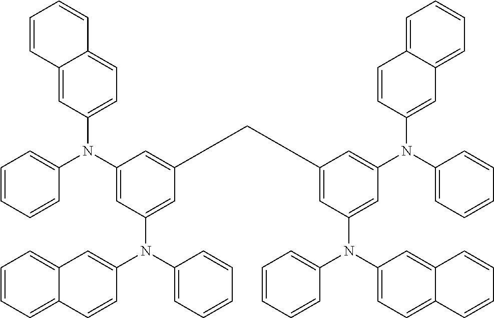 |
| (141) | 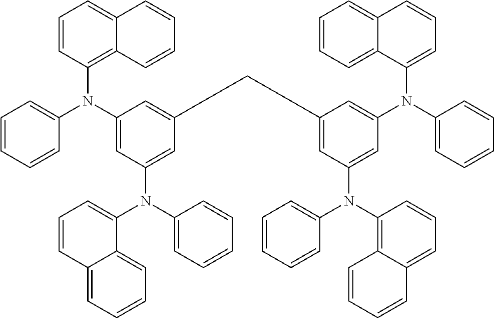 |
| (142) | 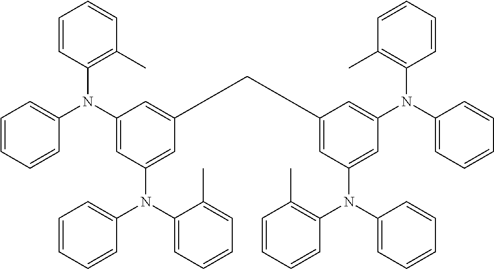 |
| (143) | 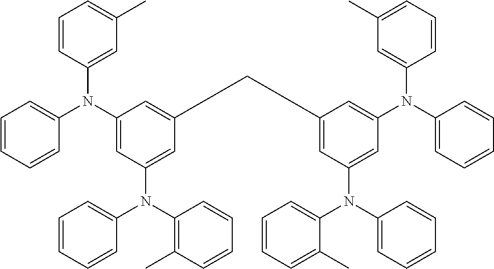 |
| (144) | 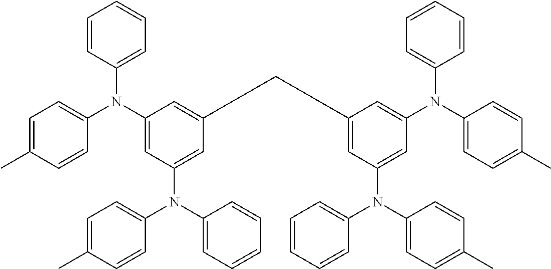 |
| (145) | 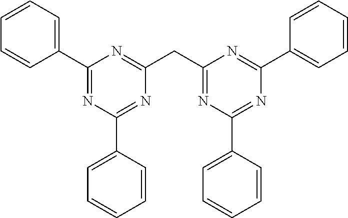 |
| (146) | 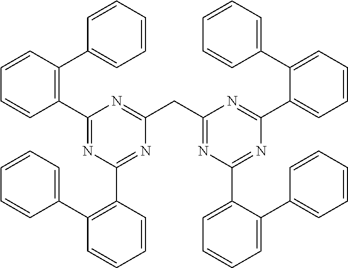 |
| (147) | 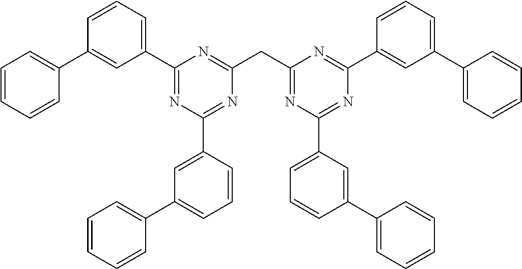 |
| (148) | 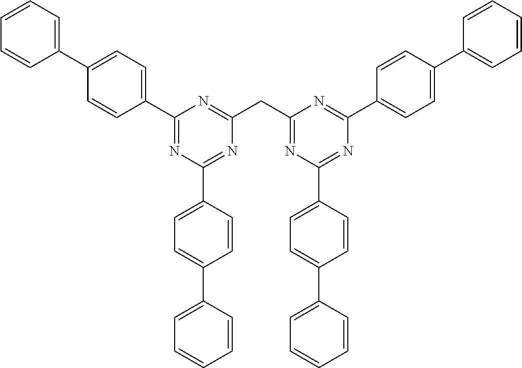 |
| (149) | 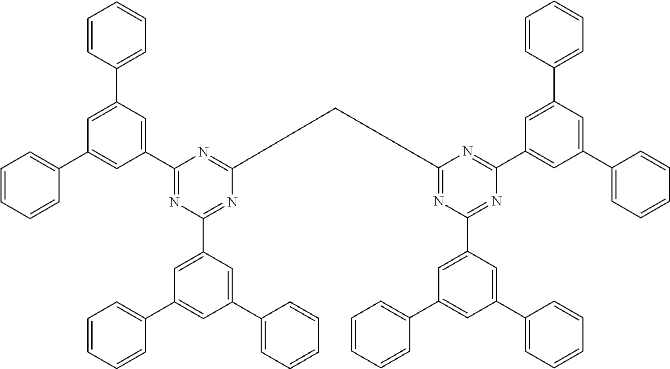 |
| (150) | 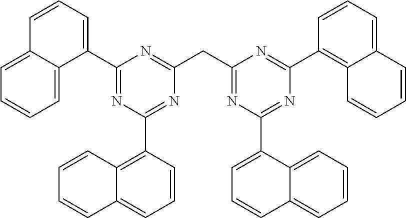 |
| (151) | 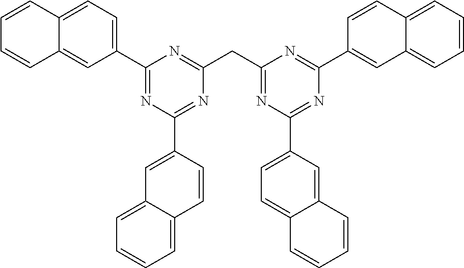 |
| (152) | 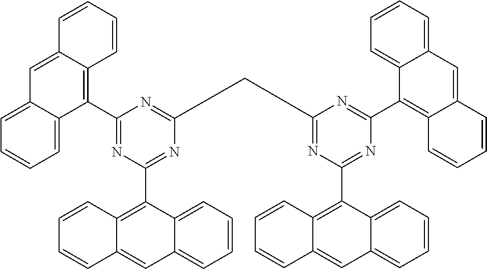 |
| (153) | 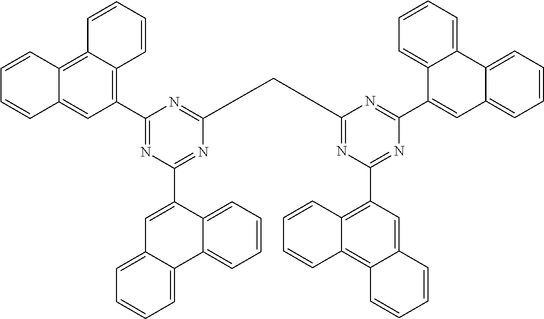 |
| (154) | 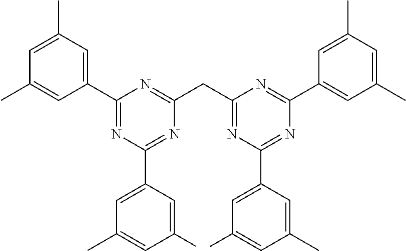 |
| (155) | 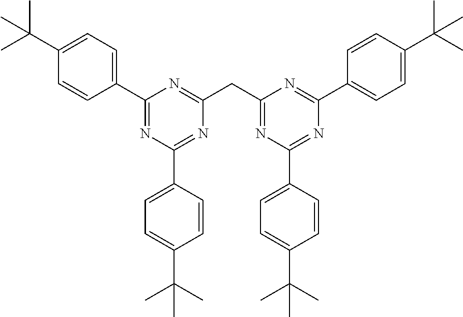 |
| (156) | 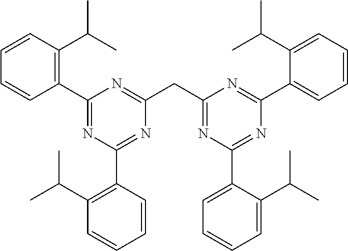 |
| (157) | 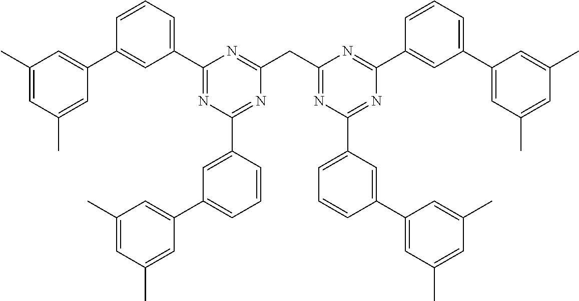 |
| (158) | 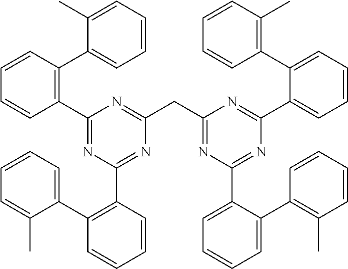 |
| (159) | 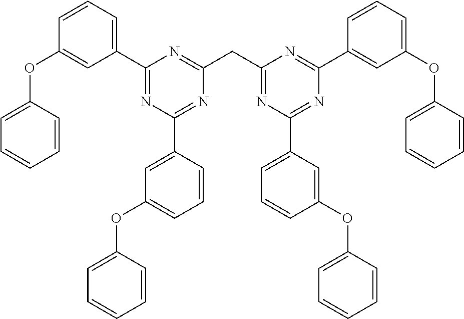 |
| (160) | 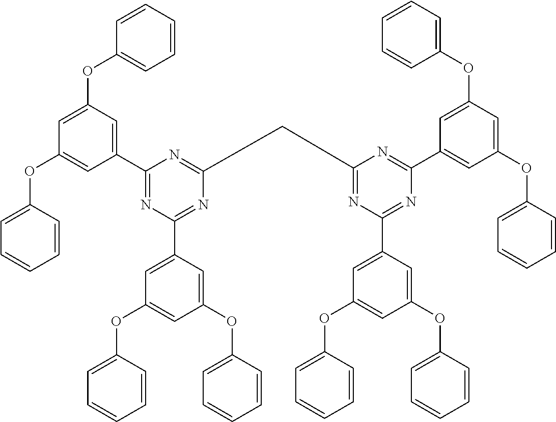 |
| (161) | 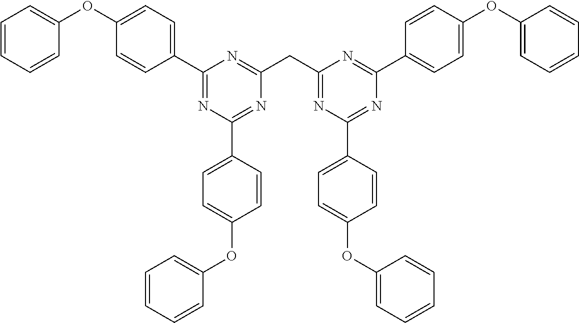 |
| (162) | 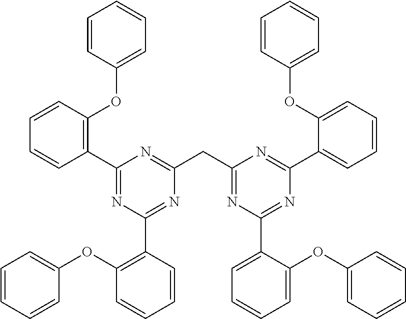 |
| (163) | 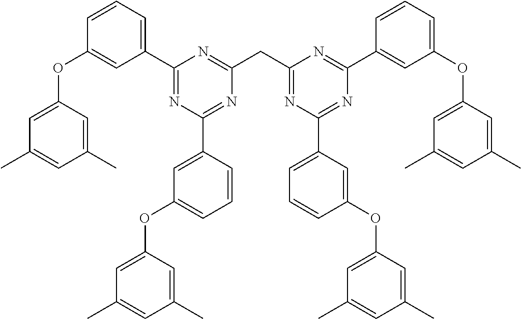 |
| (164) | 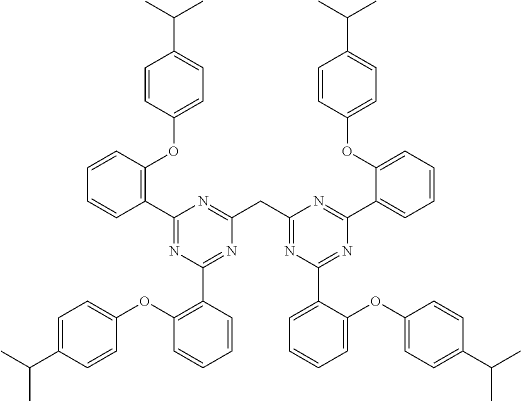 |
| (165) | 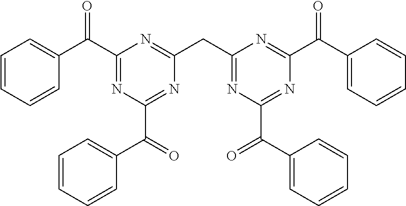 |
| (166) | 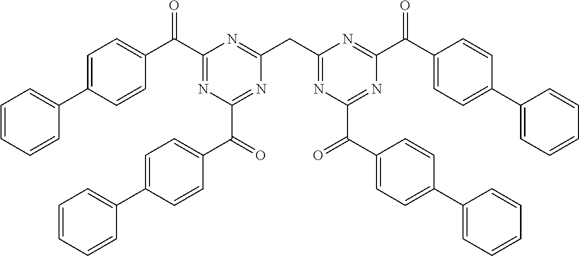 |
| (167) | 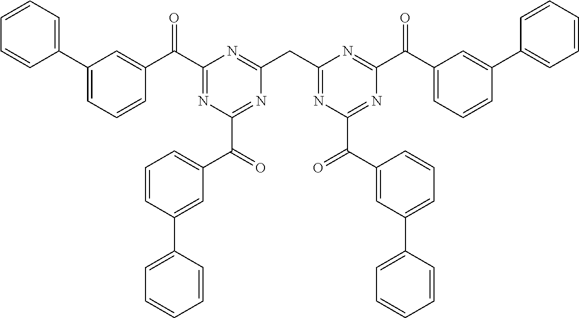 |
| (168) | 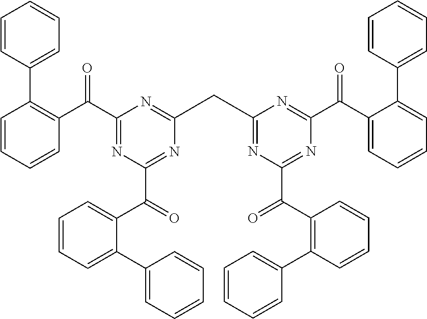 |
| (169) | 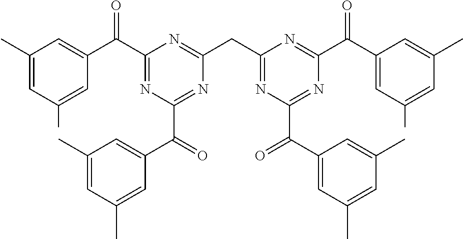 |
| (170) | 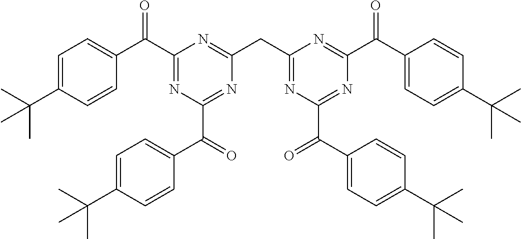 |
| (171) | 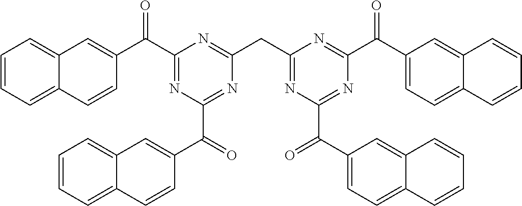 |
| (172) | 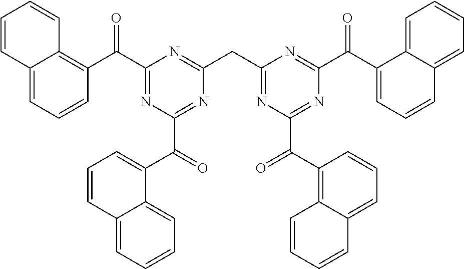 |
| (173) | 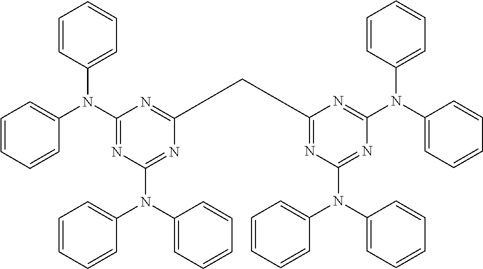 |
| (174) | 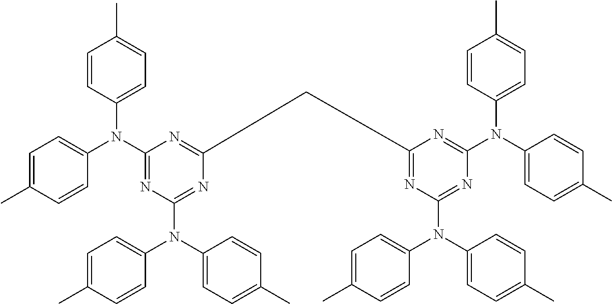 |
| (175) | 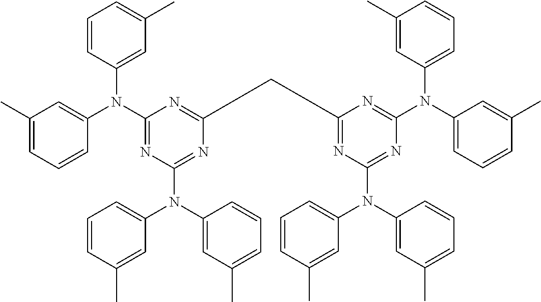 |
| (176) | 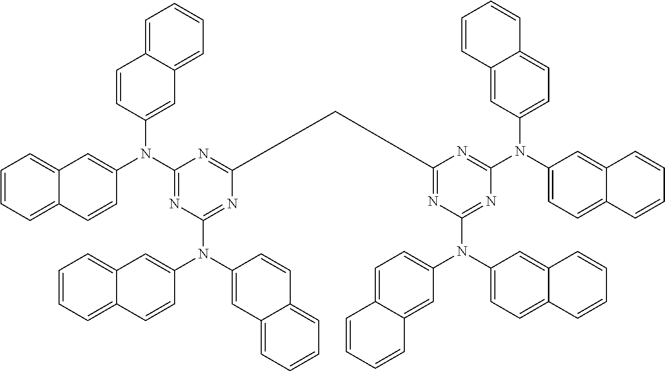 |
| (177) | 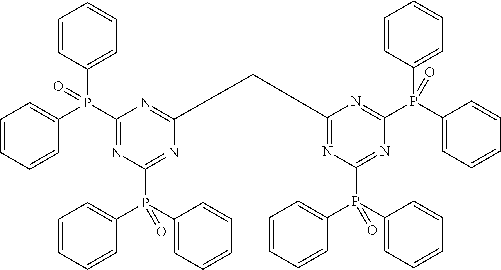 |
| (178) | 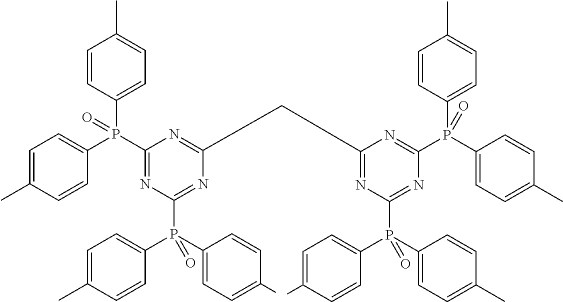 |
| (179) | 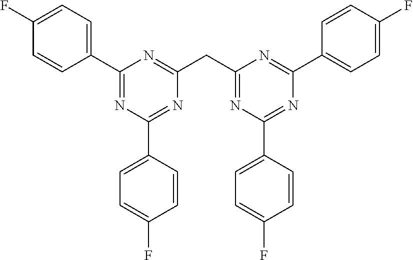 |
| (180) | 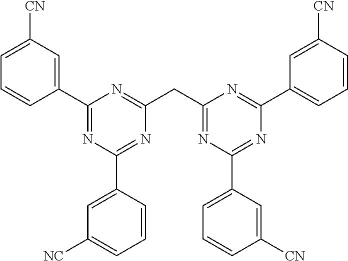 |
| (181) | 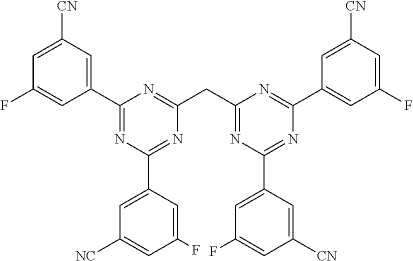 |
| (182) | 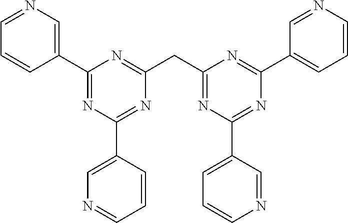 |
| (183) | 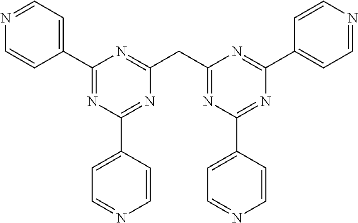 |
| (184) | 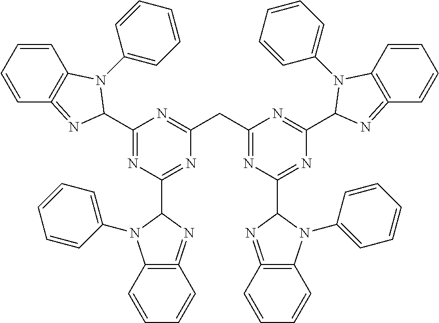 |
| (185) | 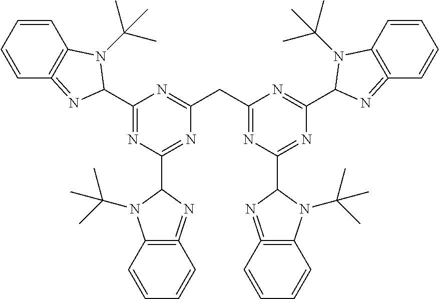 |
| (186) | 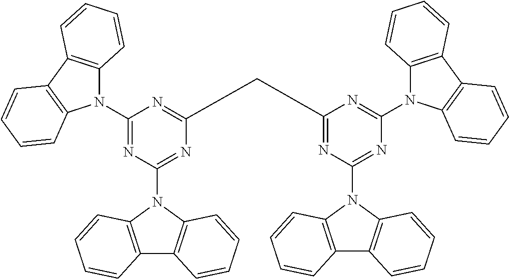 |
| (187) | 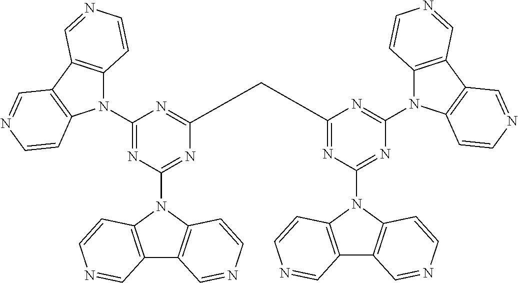 |
| (188) | 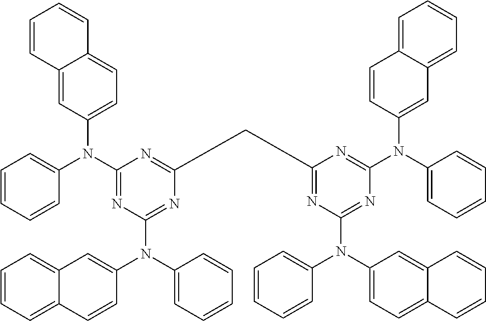 |
| (189) | 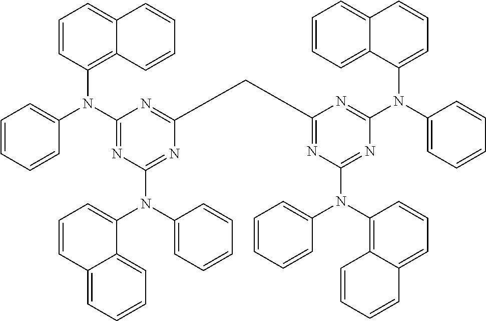 |
| (190) | 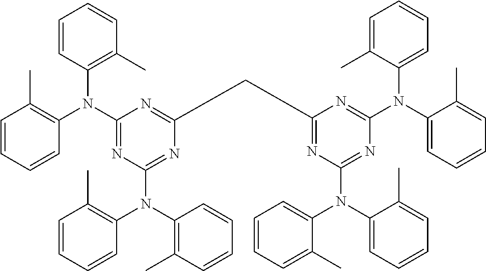 |
| (191) | 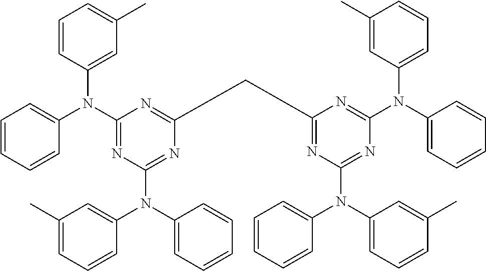 |
| (192) | 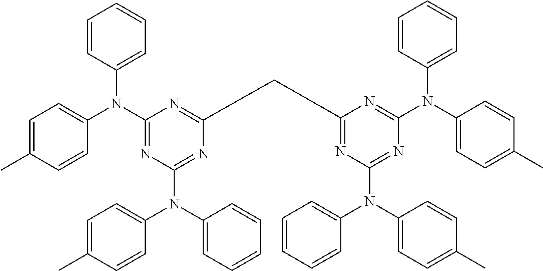 |
| (193) | 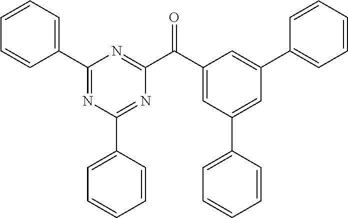 |
| (194) | 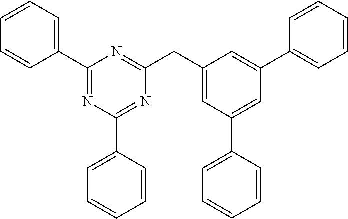 |
| (195) | 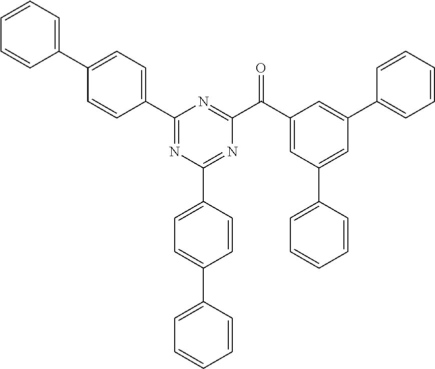 |
| (196) | 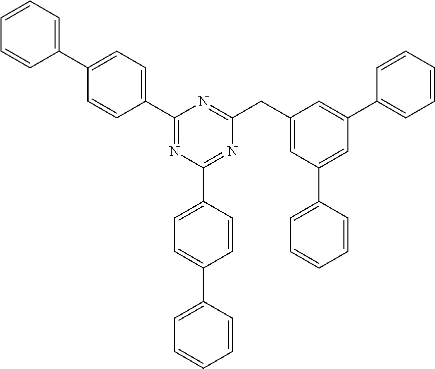 |
| (197) | 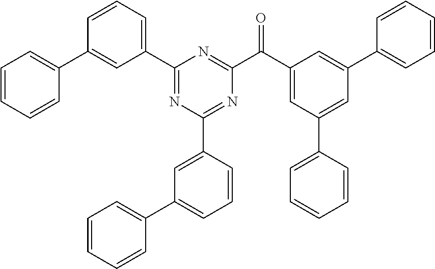 |
| (198) | 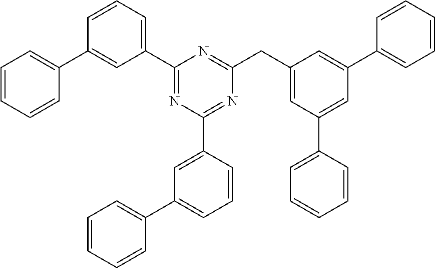 |
| (199) | 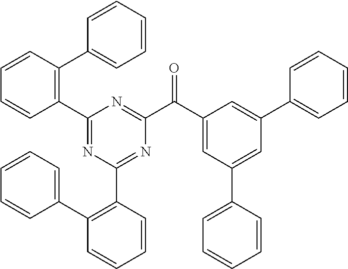 |
| (200) | 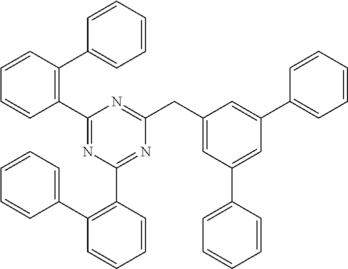 |
| (201) | 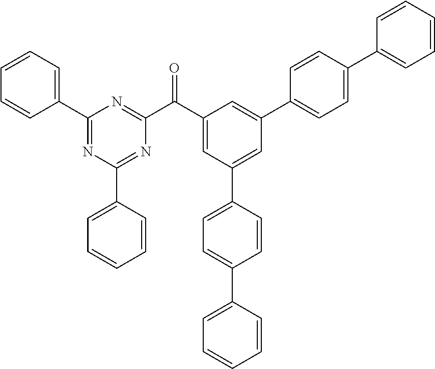 |
| (202) | 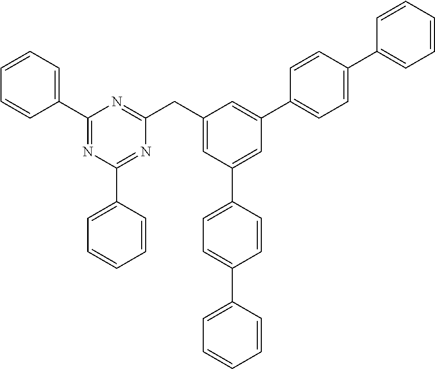 |
| (203) | 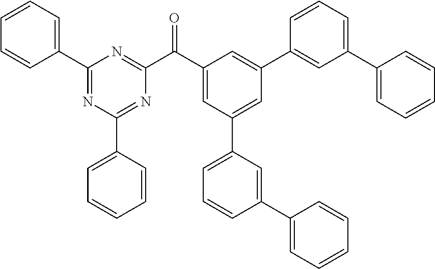 |
| (204) | 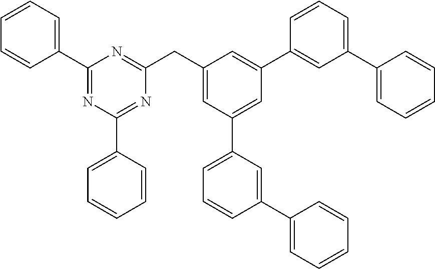 |
| (205) | 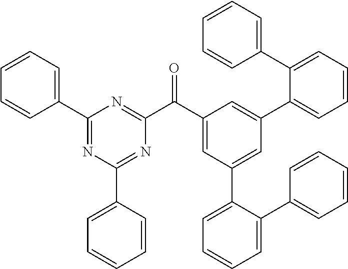 |
| (206) | 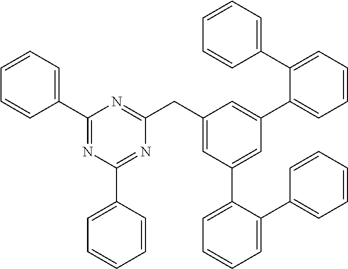 |
| (207) | 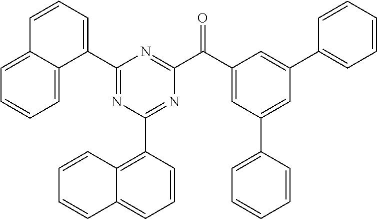 |
| (208) | 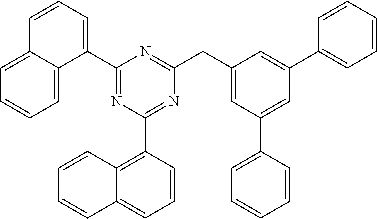 |
| (209) | 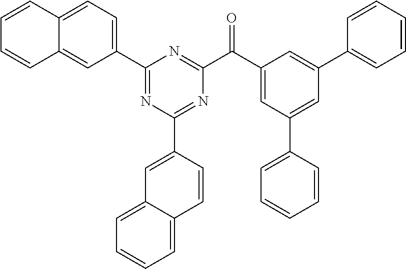 |
| (210) | 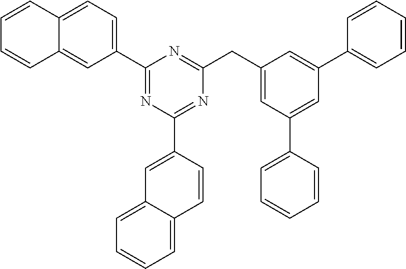 |
| (211) | 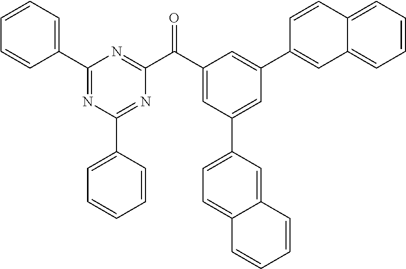 |
| (212) | 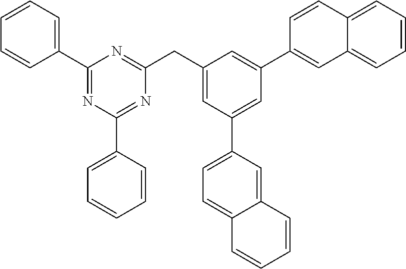 |
| (213) | 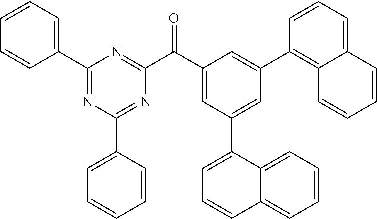 |
| (214) | 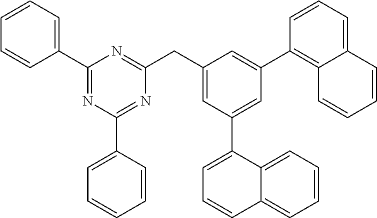 |
| (215) | 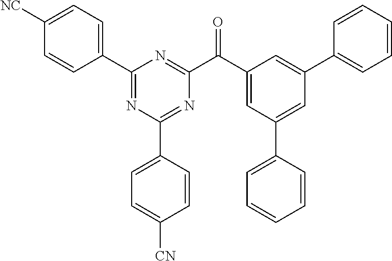 |
| (216) | 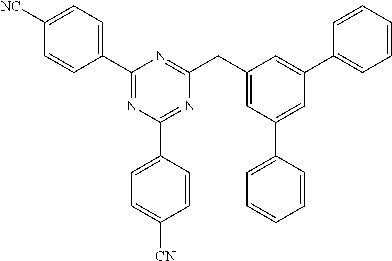 |
| (217) | 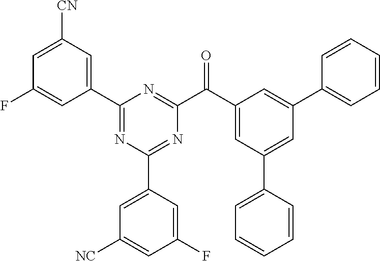 |
| (218) | 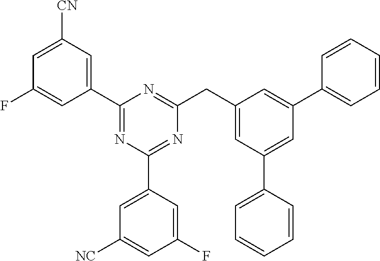 |
| (219) | 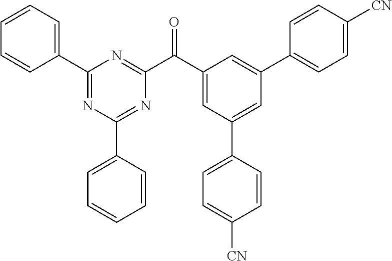 |
| (220) | 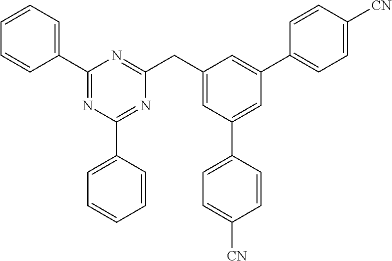 |
| (221) | 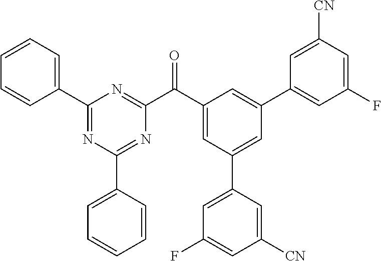 |
| (222) | 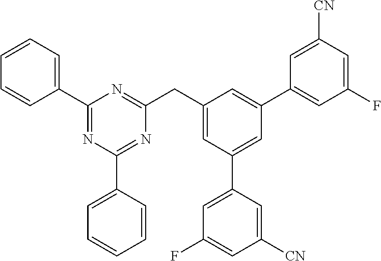 |
| (223) | 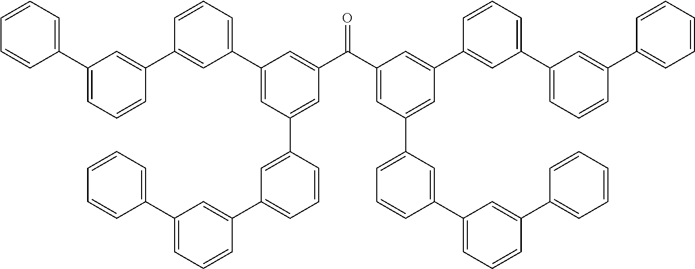 |
| (224) | 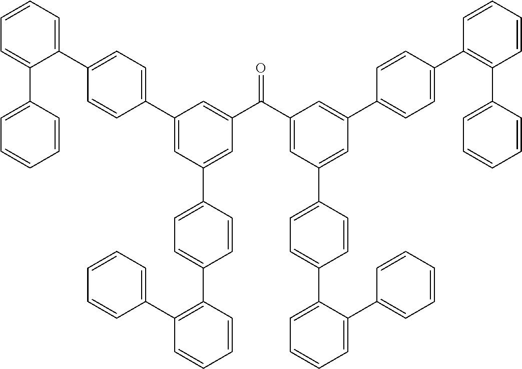 |
| (225) | 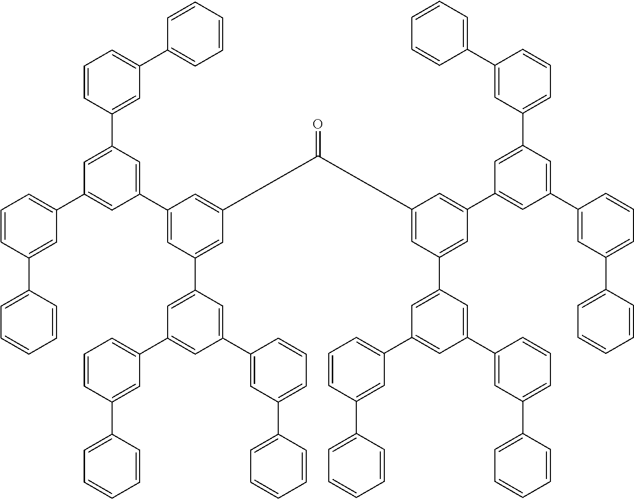 |
| (226) | 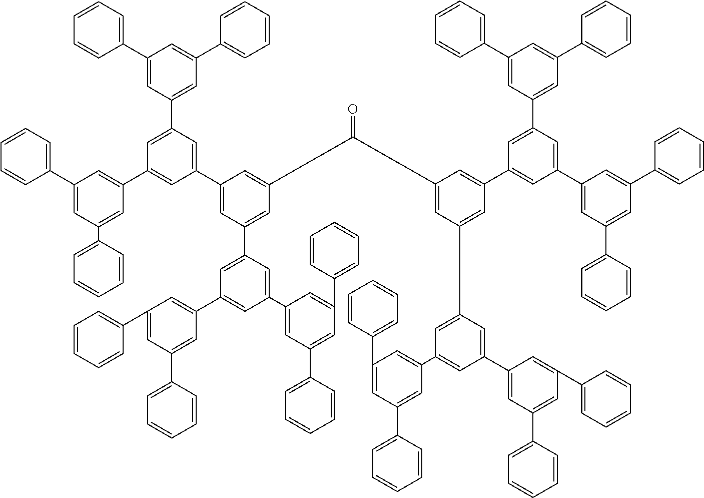 |
| (227) | 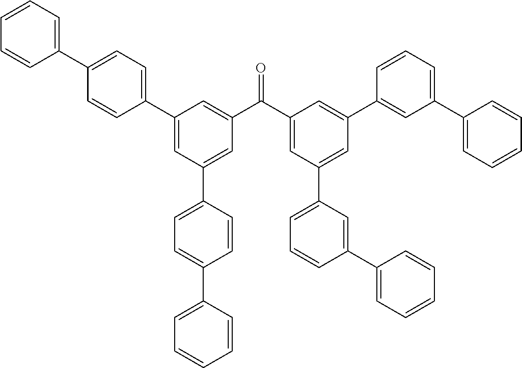 |
| (228) | 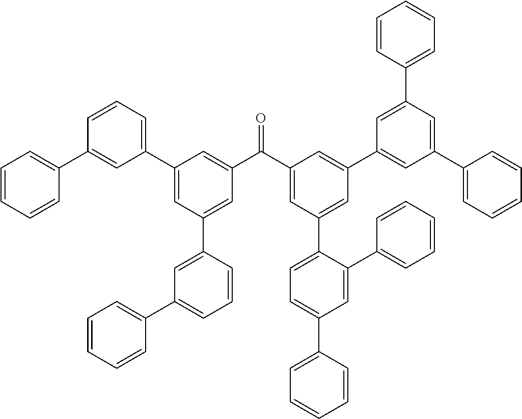 |
| (229) | 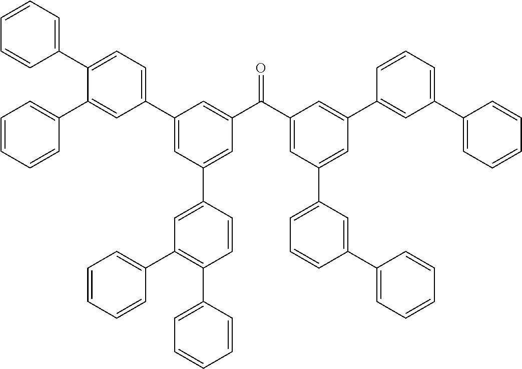 |
| (230) | 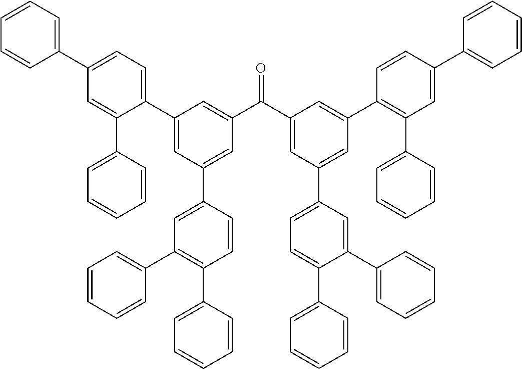 |
| (231) | 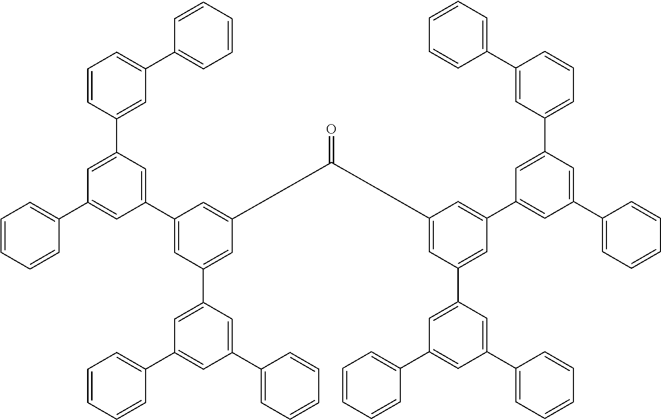 |
| (232) | 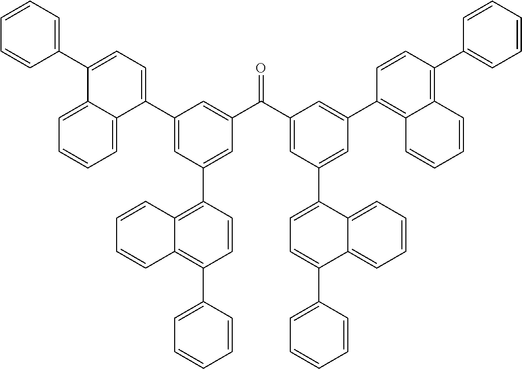 |
| (233) | 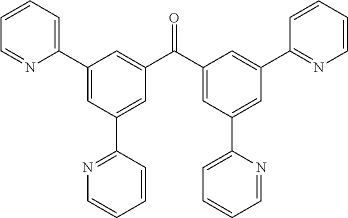 |
| (234) | 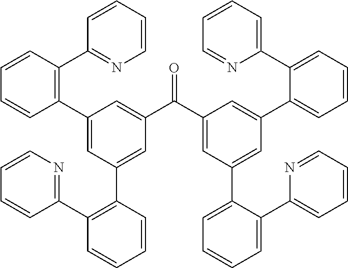 |
| (235) | 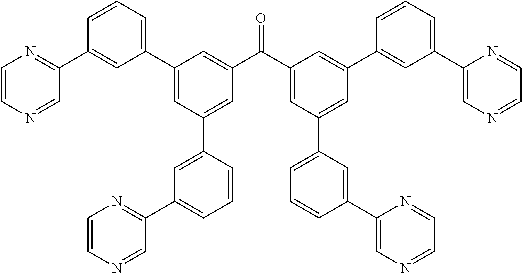 |
| (236) | 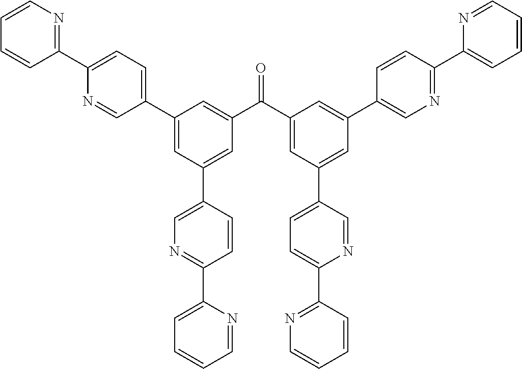 |
| (237) | 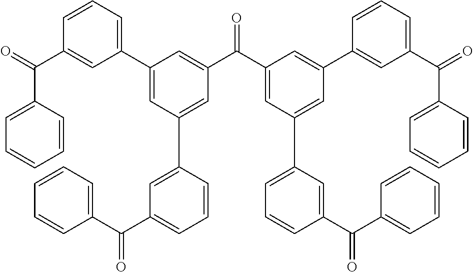 |
| (238) | 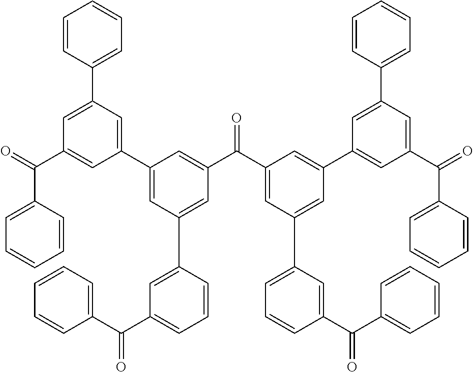 |
| (239) | 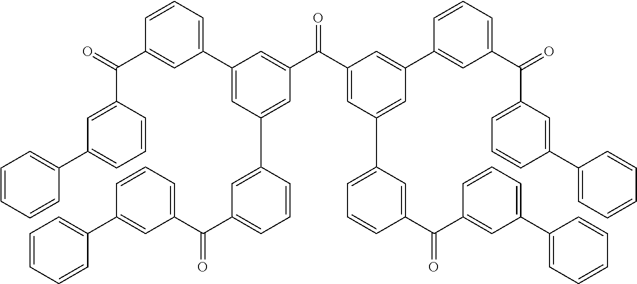 |
| (240) | 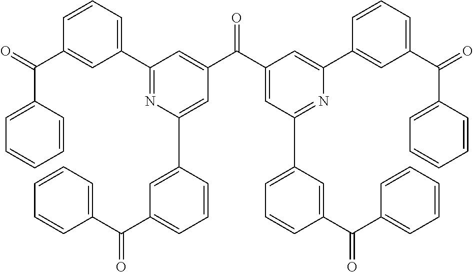 |
| (241) | 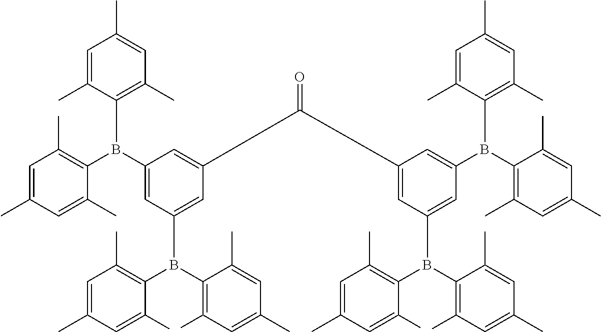 |
| (242) | 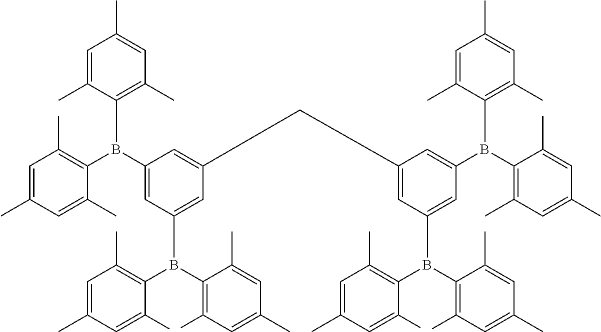 |
| (243) | 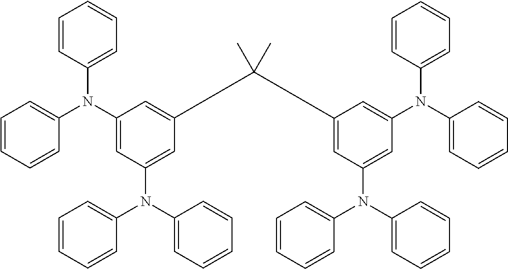 |
| (244) | 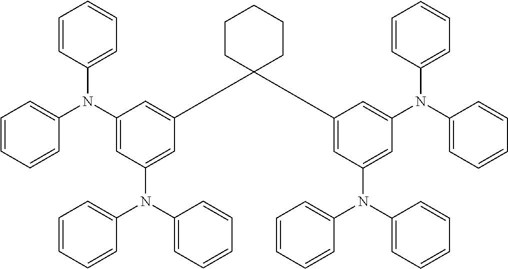 |
| (245) | 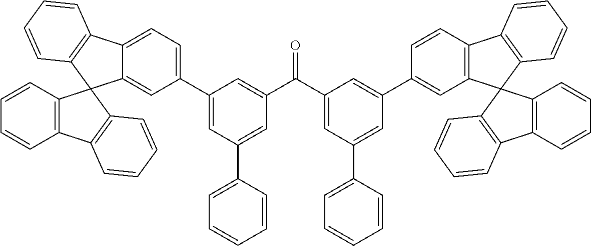 |
| (236) | 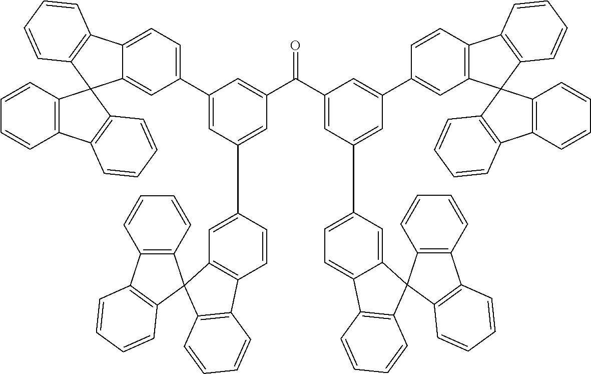 |
| (247) | 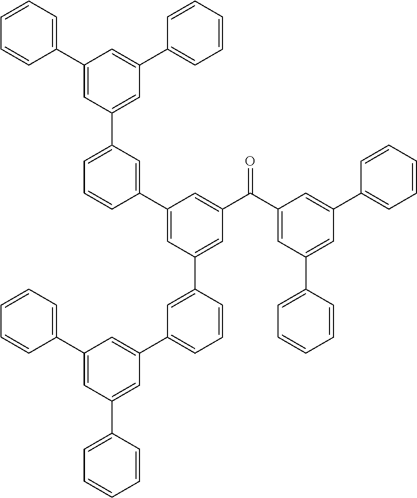 |
| (248) | 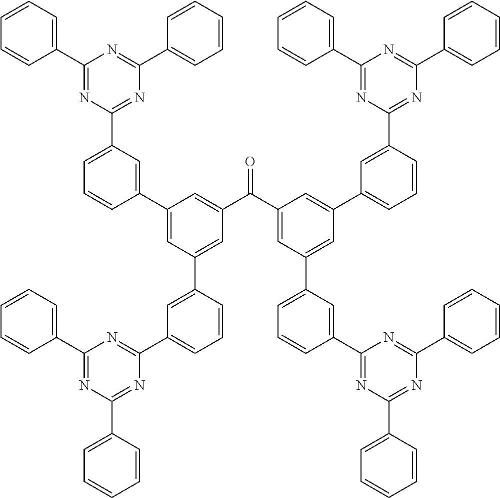 |
| (249) | 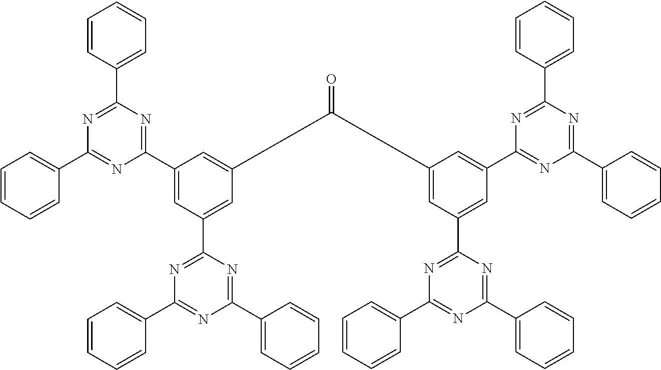 |
| (250) | 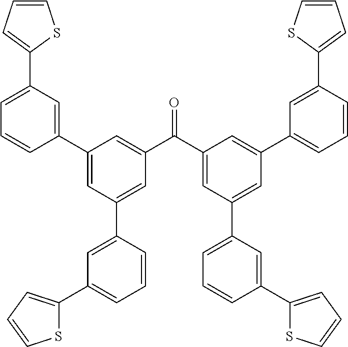 |

The compounds of the formula (1) and formulae (19) to (22) according to the invention can be prepared by the synthetic steps described below, which are generally known to the person skilled in the art. The starting compound for symmetrically substituted compounds according to the invention can be, for example, 3,3′,5,5′-tetrabromobenzophenone (Eur. J. Org. Chem. 2006, 2523-2529) or bis(3,5-dibromophenyl)methane (J. Org. Chem. 1994, 59, 7701-7703). These tetrabromides can be converted by methods known to the person skilled in the art. The palladium-catalysed reaction with boronic acids or boronic acid derivatives (Suzuki coupling) or the palladium-catalysed reaction with organozinc compounds (Negishi coupling) leads to aromatic or heteroaromatic compounds according to the invention (Scheme 1). A further process for the preparation of 3,3′,5,5′-tetrabromobenzophenone consists in the monolithiation of 1,3,5-tribromobenzene, followed by reaction with N,N-dimethylcarbamoyl chloride, as described in detail below for precursor 1.

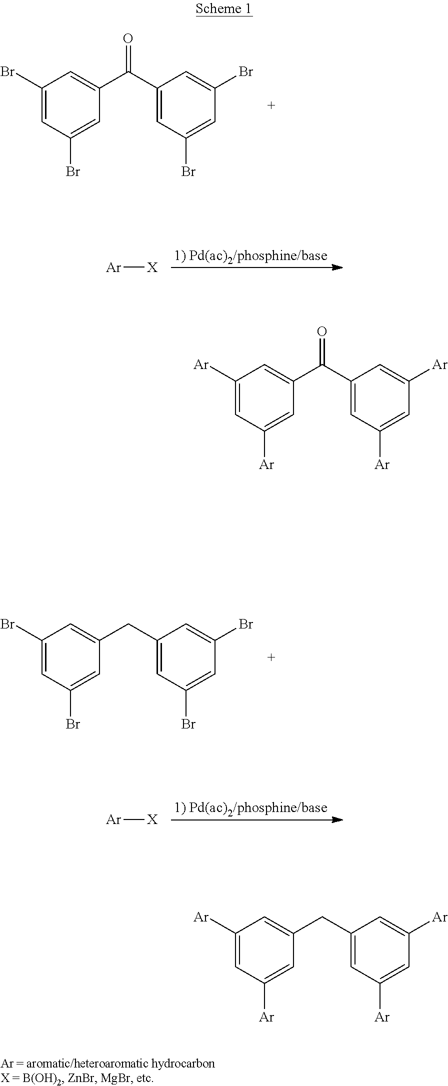

The palladium-catalysed reaction with amines (Hartwig-Buchwald coupling) leads to aromatic or heteroaromatic amines according to the invention (Scheme 2).

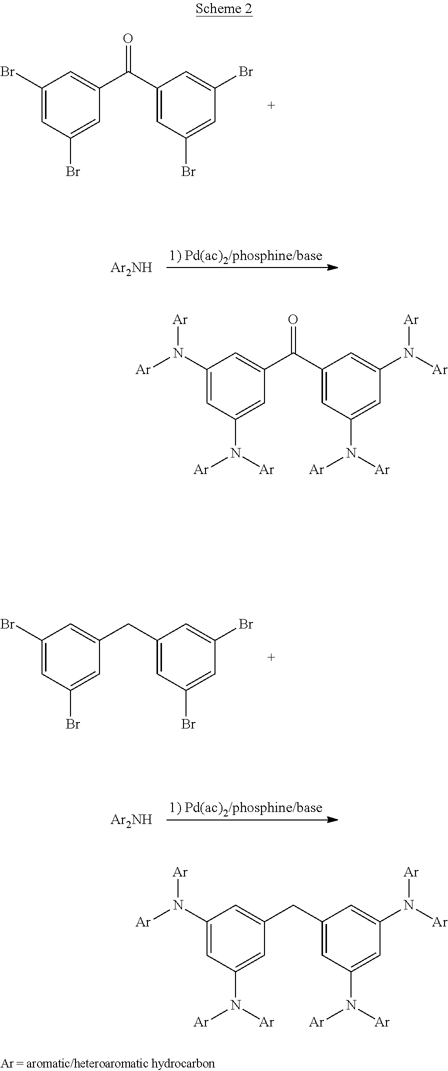

The bromine function can be converted by trans-metallation using organolithium compounds or Grignard compounds into an electrophilic group, which can then be coupled to a multiplicity of electrophiles, such as, for example, arylboron halides, aldehydes, ketones, nitriles, esters, haloesters, carbon dioxide, arylphosphine halides, halosulfinic acids, haloarylsulfonic acids, etc., where the compounds obtained in this way can be end products according to the invention or alternatively intermediates, which can be reacted further. This is illustrated by way of example with reference to the example of the preparation of a ketone according to the invention and a phosphine oxide (Scheme 3).

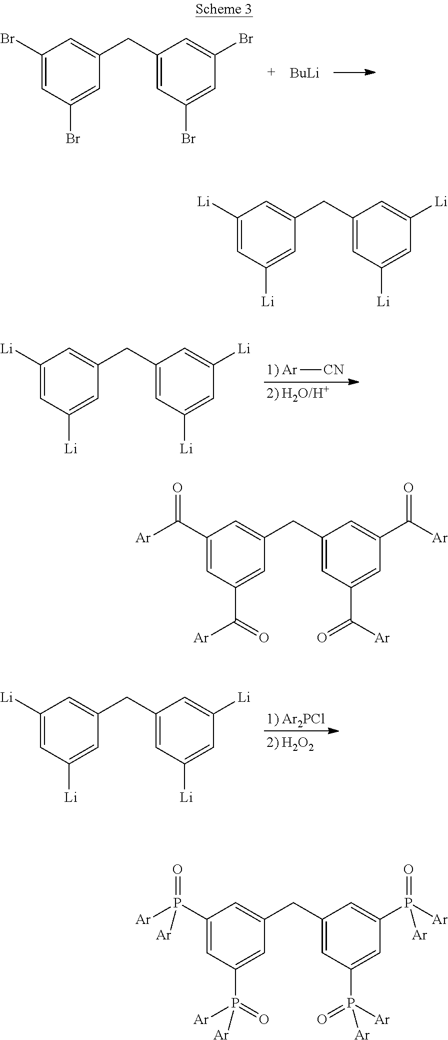

In compounds in which the group Y stands for C═O, a protecting group must firstly be introduced before the reaction with an organolithium compound or a Grignard compound and can be removed again at the end of the reaction. Suitable protecting groups for carbonyl functions are known to the person skilled in the art of organic synthesis. A suitable protecting group is, for example, 1,3-dioxolane (Scheme 4), which can be introduced by reaction with 2-chloroethanol and potassium tert-butoxide in dimethylformamide.

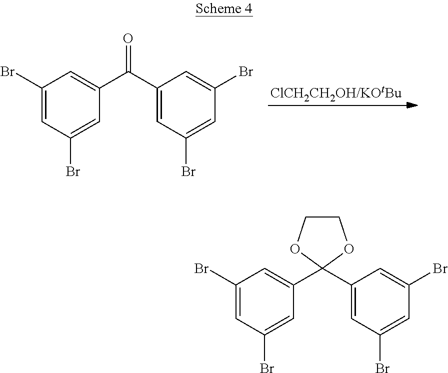

The bromine function can be converted into a nitrile function by the action of a cyanide, for example zinc cyanide, in the presence of zinc and tetrakis(triphenylphosphine)palladium in dimethylacetamide (Scheme 5).

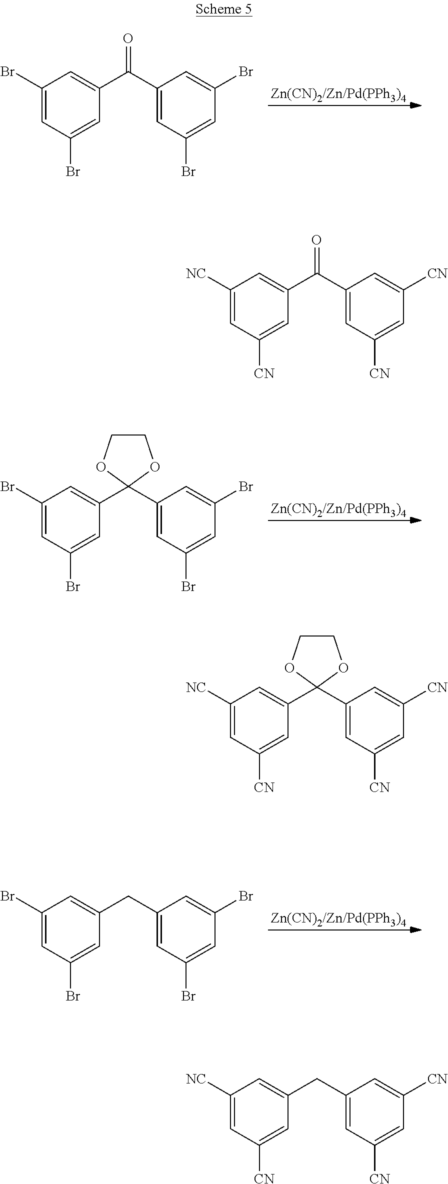

Reaction of the nitrile function with Grignard compounds and subsequent acidic hydrolysis enables the preparation of ketones according to the invention therefrom (Scheme 6).

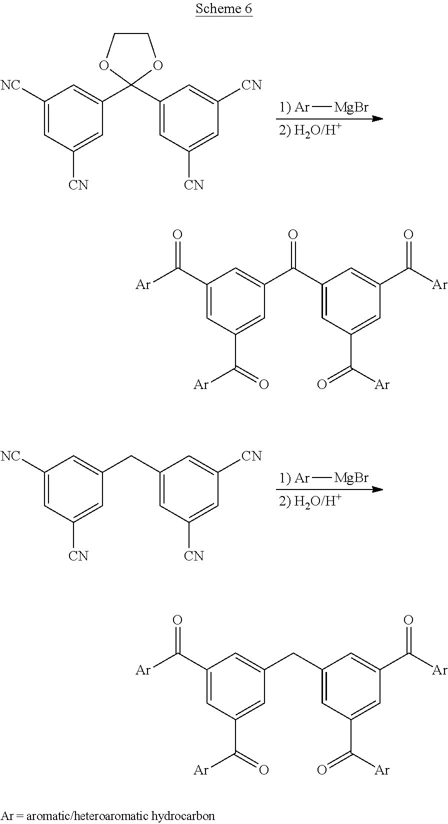

A further possibility is basic hydrolysis of the nitrile function to the carboxylic acid, which can be converted into the corresponding acid chloride by further reaction with thionyl chloride and into benzimidazole derivatives according to the invention by further reaction with suitable diamines (Scheme 7).

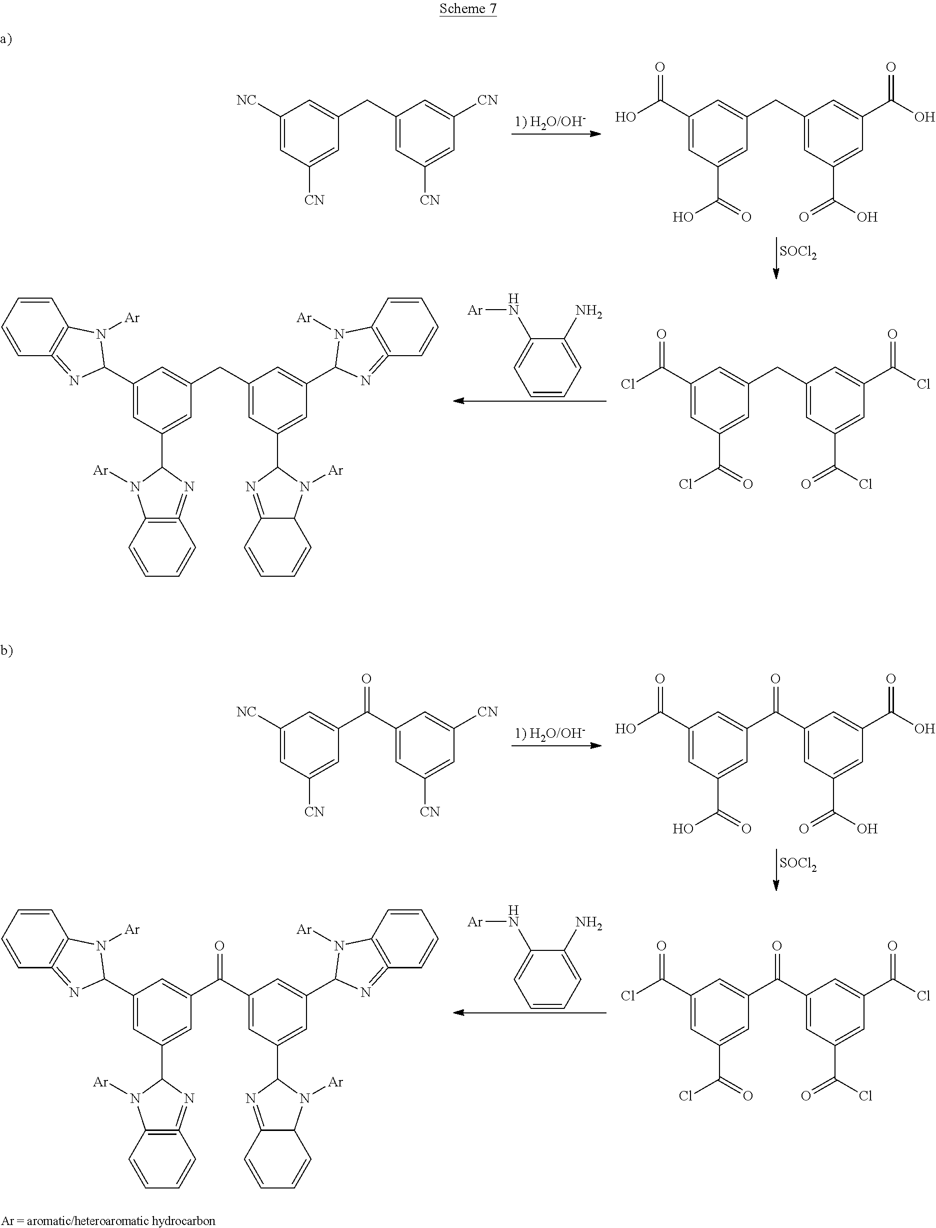

The preparation of aryl triazinyl ketones or bistriazinyl ketones can be carried out, for example, starting from cyanuric chloride, which can be converted, in accordance with EP 810453, into the 1-chloro-3,5-diaryltriazine, which can be converted consecutively by the action of magnesium in THF in accordance with U.S. Pat. No. 2,959,589 into the 3,5-diaryltriazin-1-ylmagnesium chloride, which can be reacted with suitable electrophiles, such as nitriles or carbamoyl chlorides, where acidic hydrolysis of the addition compounds obtained in this way gives the desired ketones (Scheme 8).

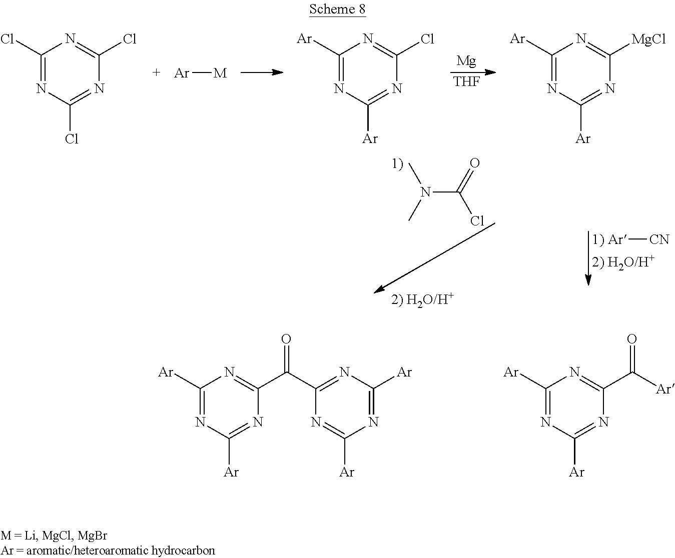

The invention furthermore relates to a process for the preparation of the compounds of the formula (1) and formulae (19) to (22) comprising the coupling of a substituted or unsubstituted bis(3,5-dibromobenzophenone) to an aromatic or heteroaromatic boronic acid or a corresponding boronic acid derivative with metal catalysis or to a primary or secondary aromatic amine with metal catalysis or to a metal cyanide with metal catalysis.

The compounds according to the invention described above, in particular compounds which are substituted by reactive leaving groups, such as bromine, iodine, triflate, tosylate, boronic acid or boronic acid ester, can be used as monomers for the preparation of corresponding dimers, trimers, tetramers, pentamers, oligomers, polymers or as the core of dendrimers.

The oligomerisation or polymerisation here is preferably carried out via the halogen functionality or the boronic acid functionality.

The invention therefore furthermore relates to dimers, trimers, tetramers, pentamers, oligomers, polymers or dendrimers containing one or more compounds of the formula (1), where one or more radicals R¹ to R⁴ represent bonds between the compounds of the formula (1) or formulae (19) to (22) in the dimer, trimer, tetramer or pentamer or bonds from the compound of the formula (1) or formulae (19) to (22) to the polymer, oligomer or dendrimer or where this bonding takes place via substituents on the groups R. For the purposes of this invention, an oligomer is taken to mean a compound which has at least six units of the formula (1) or formulae (19) to (22). The polymers, oligomers or dendrimers may be conjugated, partially conjugated or non-conjugated. The trimers, tetramers, pentamers, oligomers or polymers may be linear or branched.

For the recurring units of the formula (1) in dimers, trimers, tetramers, pentamers, oligomers and polymers, the same preferences apply as described above. Preferred recurring units are therefore again the units of the formulae (19) to (22).

For the preparation of the oligomers or polymers, the monomers according to the invention are homopolymerised or copolymerised with further monomers. Suitable and preferred comonomers are selected from fluorenes (for example in accordance with EP 842208 or WO 00/22026), spirobifluorenes (for example in accordance with EP 707020, EP 894107 or WO 06/061181), para-phenylenes (for example in accordance with WO 92/18552), carbazoles (for example in accordance with WO 04/070772 or WO 04/113468), thiophenes (for example in accordance with EP 1028136), dihydrophenanthrenes (for example in accordance with WO 05/014689), cis- or trans-indenofluorenes (for example in accordance with WO 04/041901 or WO 04/113412), ketones (for example in accordance with WO 05/040302), phenanthrenes (for example in accordance with WO 05/104264 or WO 07/017,066) or also a plurality of these units. The polymers, oligomers and dendrimers usually also contain further units, for example emitting (fluorescent or phosphorescent) units, such as, for example, vinyltriarylamines (for example in accordance with WO 07/068,325) or phosphorescent metal complexes (for example in accordance with WO 06/003000), and/or charge-transport units, in particular triarylamine derivatives.

The present invention furthermore relates to mixtures comprising at least one compound of the formula (1) or formulae (19) to (22) or a corresponding dimer, trimer, tetramer, pentamer, oligomer or polymer and at least one further compound. The further compound may be, for example, a fluorescent or phosphorescent dopant if the compound of the formula (1) or formulae (19) to (22) is used as matrix material. Suitable fluorescent and phosphorescent dopants are shown below in connection with the organic electroluminescent devices and are also preferred for the mixtures according to the invention. The further compound may also be a dopant if the compound of the formula (1) or formulae (19) to (22) is a hole-transport or electron-transport compound. Suitable dopants are shown below in connection with the organic electroluminescent devices.

The present invention again furthermore relates to solutions comprising at least one compound of the formula (1) or a corresponding dimer, trimer, tetramer, pentamer, oligomer or polymer and at least one organic solvent.

Solutions of this type are necessary for the production of the organic electronic device from solution, for example by spin coating or by printing processes.

The compounds of the formula (1) or formulae (19) to (22) according to the invention and corresponding dimers, trimers, tetramers, pentamers, oligomers, polymers or dendrimers are suitable for use in electronic devices, in particular in organic electroluminescent devices (OLEDs, PLEDs), organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electrochemical cells (LECs), organic laser diodes (O-lasers) or organic photoreceptors.

Depending on the substitution, the compounds are employed in different functions and layers.

The invention therefore furthermore relates to the use of compounds of the formula (1) or formulae (19) to (22) or corresponding dimers, trimers, tetramers, pentamers, oligomers, polymers or dendrimers in electronic devices, in particular in organic electroluminescent devices. The preferred embodiments mentioned above also apply to the use of compounds in organic electronic devices.

The invention again furthermore relates to electronic devices, in particular the electronic devices mentioned above, comprising at least one compound of the formula (1) or formulae (19) to (22) or a corresponding dimer, trimer, tetramer, pentamer, oligomer, polymer or dendrimer, in particular organic electroluminescent devices, comprising anode, cathode and at least one emitting layer, characterised in that at least one organic layer, which may be an emitting layer or another layer, comprises at least one compound of the formula (1) or formulae (19) to (22) or a corresponding dimer, trimer, tetramer, pentamer, oligomer, polymer or dendrimer. The preferred embodiments mentioned above also apply to organic electronic devices.

Apart from cathode, anode and the emitting layer, the organic electroluminescent device may also comprise further layers. These are selected, for example, from in each case one or more hole-injection layers, hole-transport layers, hole-blocking layers, electron-transport layers, electron-injection layers, electron-blocking layers, exciton-blocking layers, charge-generation layers and/or organic or inorganic p/n junctions. Furthermore, the layers, in particular the charge-transport layers, may also be doped. The doping of the layers may be advantageous for improved charge transport. However, it should be pointed out that each of these layers does not necessarily have to be present, and the choice of layers is always dependent on the compounds used and in particular also on whether the electroluminescent device is fluorescent or phosphorescent.

In a further preferred embodiment of the invention, the organic electroluminescent device comprises a plurality of emitting layers, where at least one organic layer comprises at least one compound of the formula (1) or formulae (19) to (22). These emission layers particularly preferably have in total a plurality of emission maxima between 380 nm and 750 nm, resulting overall in white emission, i.e. various emitting compounds which are able to fluoresce or phosphoresce and which emit blue and yellow, orange or red light are used in the emitting layers. Particular preference is given to three-layer systems, i.e. systems having three emitting layers, where at least one of these layers comprises at least one compound of the formula (1) or formulae (19) to (22) and where the three layers exhibit blue, green and orange or red emission (for the basic structure, see, for example, WO 05/011013). Emitters which have broad-band emission bands and thus exhibit white emission are likewise suitable for white emission.

In a preferred embodiment of the invention, the compounds of the formula (1) or formulae (19) to (22) are employed as matrix material for fluorescent or phosphorescent compounds in an emitting layer. In a matrix material for phosphorescent compounds, Y preferably stands for C═O and/or one or more groups R stand for C(═O)Ar, S(═O)Ar, S(═O)₂Ar, P(═O)Ar₂ or N-carbazolyl. In particular, the group Y stands for C═O. In a matrix material for fluorescent compounds, one or more groups R preferably stand for an aromatic or heteroaromatic ring system, in particular for an aromatic ring system containing anthracene. The group Y then preferably stands for C(R¹)₂.

A matrix material in a system comprising matrix and dopant is taken to mean the component which is present in the higher proportion in the system. In a system comprising a matrix and a plurality of dopants, the matrix is taken to mean the component whose proportion in the mixture is the highest. It is also possible to use a mixture of a plurality of matrix materials.

If the compound of the formula (1) or formulae (19) to (22) is employed as matrix material for an emitting compound in an emitting layer, it can be employed in combination with one or more phosphorescent materials (triplet emitters). For the purposes of this invention, phosphorescence is taken to mean the luminescence from an excited state of relatively high spin multiplicity, i.e. a spin state >1, in particular from an excited triplet state. For the purposes of the present invention, all luminescent iridium and platinum complexes, in particular, are intended to be taken to mean phosphorescent compounds. The mixture of the compound of the formula (1) and the emitting compound then comprises between 99 and 50% by vol., preferably between 98 and 50% by vol., particularly preferably between 97 and 60% by vol., in particular between 95 and 85% by vol., of the compound of the formula (1), based on the entire mixture of emitter and matrix material. Correspondingly, the mixture comprises between 1 and 50% by vol., preferably between 2 and 50% by vol., particularly preferably between 3 and 40% by vol., in particular between 5 and 15% by vol., of the emitter, based on the entire mixture of emitter and matrix material.

Preference is also given to the use of a plurality of matrix materials as mixture. In particular, one component of this mixture is a hole-transporting compound and a further component of the mixture is an electron-transporting compound. If the compound of the formula (1) or formulae (19) to (22) is an electron-transporting compound, i.e. if, for example, the group Y stands for C═O and/or the groups R stand for C(═O)Ar or for aromatic or heteroaromatic ring systems, this compound is preferably used in combination with a hole-transporting matrix material. Preferred hole-conducting matrix materials are triarylamines and carbazole derivatives. If the compound of the formula (1) or formulae (19) to (22) is a hole-conducting cornpound, i.e. if, for example, the group Y stands for C(R¹)₂ and the groups R stand for N(Ar)₂, this compound is preferably used in combination with an electron-transporting matrix material. Preferred electron-transporting matrix materials are aromatic ketones, aromatic phosphine oxides, aromatic sulfoxides, aromatic sulfones and triazine derivatives.

Suitable phosphorescent compounds (=triplet emitters) are, in particular, compounds which emit light, preferably in the visible region, on suitable excitation and in addition contain at least one atom having an atomic number of greater than 20, preferably greater than 38 and less than 84, particularly preferably greater than 56 and less than 80. The phosphorescence emitters used are preferably compounds which contain copper, molybdenum, tungsten, rhenium, ruthenium, osmium, rhodium, iridium, palladium, platinum, silver, gold or europium, in particular compounds which contain iridium or platinum.

Examples of the emitters described above are revealed by the applications WO 00/70655, WO 01/41512, WO 02/02714, WO 02/15645, EP 1191613, EP 1191612, EP 1191614 and WO 05/033244. In general, all phosphorescent complexes as used in accordance with the prior art for phosphorescent OLEDs and as are known to the person skilled in the art in the area of organic electroluminescence are suitable, and the person skilled in the art will be able to use further phosphorescent complexes without inventive step.

If the compound of the formula (1) or formulae (19) to (22) is employed as matrix material for fluorescent compounds, the proportion of matrix material in the emitting layer is between 50.0 and 99.9% by vol., preferably between 80.0 and 99.5% by vol., particularly preferably between 90.0 and 99.0% by vol. Correspondingly, the proportion of the dopant is between 0.1 and 50.0% by vol., preferably between 0.1 and 20.0% by vol., particularly preferably between 0.5 and 15% by vol., very particularly preferably between 1.0 and 10.0% by vol.

Preferred dopants are selected from the class of the monostyrylamines, the distyrylamines, the tristyrylamines, the tetrastyrylamines, the styrylphosphines, the styryl ethers and the arylamines. A monostyrylamine is taken to mean a compound which contains one substituted or unsubstituted styryl group and at least one, preferably aromatic, amine. A distyrylamine is taken to mean a compound which contains two substituted or unsubstituted styryl groups and at least one, preferably aromatic, amine. A tristyrylamine is taken to mean a compound which contains three substituted or unsubstituted styryl groups and at least one, preferably aromatic, amine. A tetrastyrylamine is taken to mean a compound which contains four substituted or unsubstituted styryl groups and at least one, preferably aromatic, amine. The styryl groups are particularly preferably stilbenes, which may also be further substituted. Corresponding phosphines and ethers are defined analogously to the amines. For the purposes of this invention, an arylamine or an aromatic amine is taken to mean a compound which contains three substituted or unsubstituted aromatic or heteroaromatic ring systems bonded directly to the nitrogen. At least one of these aromatic or heteroaromatic ring systems is preferably a condensed ring system, preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 2-position or in the 9-position. An aromatic anthracenediamine is taken to mean a compound in which two diarylamino groups are bonded directly to an anthracene group, preferably in the 2,6-position or in the 9,10-position. Aromatic pyrenamines, pyrenediamines, chrysenamines and chrysenediamines are defined analogously thereto, where the diarylamino groups on the pyrene are preferably bonded in the 1-position or in the 1,6-position. Further preferred dopants are selected from indenofluorenamines or indenofluorenediamines, for example in accordance with WO 06/122630, benzoindenofluorenamines or benzoindenofluorenediamines, for example in accordance with WO 08/006,449, and dibenzoindenofluorenamines or dibenzoindenofluorenediamines, for example in accordance with WO 07/140,847. Examples of dopants from the class of the styrylamines are substituted or unsubstituted tristilbenamines or the dopants described in WO 06/000388, WO 06/058737, WO 06/000389, WO 07/065,549 and WO 07/115,610.

In a further embodiment of the invention, the compounds of the formula (1) or formulae (19) to (22) are employed as hole-transport material or as hole-injection material or as electron-blocking material or as exciton-blocking material. The group Y then preferably stands for C(R¹)₂. The compounds are then preferably substituted by at least one N(Ar)₂ group, preferably by at least two N(Ar)₂ groups, and/or contain further groups which improve hole transport. All groups R here particularly preferably stand for N(Ar)₂. The N(Ar)₂ groups are preferably selected from the formulae (17) and (18) described above. Further preferred groups which improve hole transport are, for example, electron-rich heteroaromatic compounds, in particular thiophene, pyrrole or furan as group R. The compound is preferably employed in a hole-transport or hole-injection or electron-blocking or exciton-blocking layer. For the purposes of this invention, a hole-injection layer is a layer which is directly adjacent to the anode. For the purposes of this invention, a hole-transport layer is a layer which is located between a hole-injection layer and an emission layer. For the purposes of this invention, an electron-blocking or exciton-blocking layer is a layer which is directly adjacent to an emitting layer on the anode side. If the compounds of the formula (1) are used as hole-transport or hole-injection material, it may be preferred for them to be doped with electronacceptor compounds, for example with F₄-TCNQ or with compounds as described in EP 1476881 or EP 1596445.

In still a further embodiment of the invention, the compounds of the formula (1) or formulae (19) to (22) are employed as electron-transport material or as hole-blocking material in an electron-transport layer or a hole-blocking layer respectively. It is preferred here for the group Y to stand for C═O and/or for at least one of the aromatic groups to represent a triazine, i.e. compounds of the formulae (20) and (21), and/or for at least one of the substituents R to stand for a heteroaryl group which represents an electron-deficient heterocycle, such as, for example, imidazole, pyrazole, thiazole, benzimidazole, benzothiazole, triazole, oxadiazole, benzothiadiazole, pyridine, pyrazine, pyrimidine, pyridazine, triazine, bipyridine, quinoline, isoquinoline, quinoxaline, phenanthroline, etc., or for C(═O)Ar, P(═O)Ar₂, S(═O)Ar or S(O)₂Ar. It may furthermore be preferred for the compound to be doped in the electron-transport layer, for example with electron-donor compounds or lithium salts, such as, for example, Liq. For the purposes of this invention, a hole-blocking layer is a layer which is located between an emitting layer and an electron-transport layer and is directly adjacent to the emitting layer.

Also in polymers, recurring units of the formula (1) or formulae (19) to (22) can either be employed as polymer backbone, as hole-transporting unit and/or as electron-transporting unit. The preferred substitution patterns here correspond to those described above.

Preference is furthermore given to an organic electroluminescent device, characterised in that one or more layers are applied by means of a sublimation process, in which the materials are vapour-deposited in vacuum sublimation units at an initial pressure of less than 10⁻⁵ mbar, preferably less than 10⁻⁶ mbar. However, it should be noted that the pressure may also be even lower, for example less than 10⁻⁷ mbar.

Preference is likewise given to an organic electroluminescent device, characterised in that one or more layers are applied by means of the OVPD (organic vapour phase deposition) process or with the aid of carrier-gas sublimation, in which the materials are applied at a pressure between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett. 2008, 92, 053301).

Preference is furthermore given to an organic electroluminescent device, characterised in that one or more layers are applied from solution, such as, for example, by spin coating, or by means of any desired printing process, such as, for example, screen printing, flexographic printing or offset printing, but particularly preferably LITI (light induced thermal imaging, thermal transfer printing) or ink-jet printing. Soluble compounds are necessary for this purpose. High solubility can be achieved through suitable substitution of the compounds. It is possible here to apply not only solutions of individual materials, but also solutions which comprise a plurality of compounds, for example matrix materials and dopants.

The organic electroluminescent device may also be produced as a hybrid system by applying one or more layers from solution and vapour-depositing one or more other layers. Since the compounds of the formula (1) or formulae (19) to (22) generally have high solubility in organic solvents, these compounds can be processed well from solution. Thus, for example, it is possible to apply an emitting layer comprising a compound of the formula (1) or formulae (19) to (22) and a fluorescent or phosphorescent dopant from solution and to apply a hole-blocking layer and/or an electron-transport layer on top by vacuum vapour deposition.

These processes are generally known to the person skilled in the art and can be applied by him without problems to organic electroluminescent devices comprising compounds of the formula (1) or formulae (19) to (22) or the preferred embodiments mentioned above.

The compounds according to the invention have the following surprising advantages over the prior art on use in organic electroluminescent devices:
1. The compounds according to the invention have high thermal stability and can be sublimed without decomposition.
2. On use as matrix material for phosphorescent emitters, the compounds according to the invention in which Y stands for C═O, in particular those in which the radicals R each stand for an aromatic or heteroaromatic ring system, result in a considerable improvement in the lifetime and a slight improvement in the efficiency of the organic electroluminescent device.
3. On use in an electron/exciton-blocking layer in a phosphorescent electroluminescent device, the compounds according to the invention, in particular those which contain diarylamino substituents as groups R, result in a considerable improvement in the efficiency compared with materials in accordance with the prior art. This applies, in particular, to compounds in which Y stands for C(R¹)₂.
4. The compounds according to the invention, in particular those which are substituted by diarylamino groups as groups R and/or which are substituted by electron-rich heteroaromatic groups, are very highly suitable for use as hole-injection and hole-transport material and result in a reduction in the operating voltage.

The invention is described in greater detail by the following examples, without wishing to restrict it thereby. The person skilled in the art, without being inventive, will be able to prepare further compounds according to the invention and use them in organic electronic devices.

### EXAMPLES

The following syntheses are—unless indicated otherwise—carried out under a protective-gas atmosphere in dried solvents. Solvents and reagents can be purchased from ALDRICH or ABCR.

The precursors can be prepared as follows (precursors 1-13):

### Precursor 1: Synthesis of bis(3,5-dibromophenyl)ketone

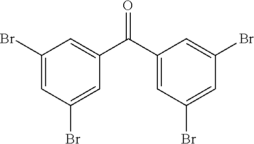

A suspension of 252.0 g (801 mmol) of 1,3,5-tribromobenzene in 4000 ml of diethyl ether is cooled to −72° C. 320 ml (800 mmol) of a solution of n-butyllithium (2.5 M in hexane) are added dropwise at such a rate that the temperature does not exceed −60° C., and the mixture is then stirred for a further one hour. A mixture of 38.8 ml (399 mmol) of N,N-dimethylcarbamoyl chloride and 80 ml of diethyl ether is subsequently added, and the mixture is stirred for a further one hour, and the cooling is then removed. From an internal temperature of 0° C., a mixture of 1000 ml of water and 80 ml of acetic acid is added dropwise over the course of one hour. The mixture is stirred for a further two hours and left to stand overnight, during which a solid deposits. 3000 ml of diethyl ether are removed by suction, and the solid is then filtered off with suction, washed once with 100 ml of ethanol, twice with 100 ml of ethanol/water (1:1), twice with 100 ml of ethanol and dried in vacuo. Yield: 168.5 g (338 mmol), 84.9%, purity about 98.5% (HPLC).

### Precursor 2: Synthesis of bis(3,5-dibromophenyl)methanol

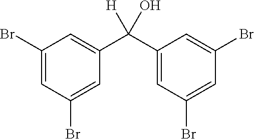

Bis(3,5-dibromophenyl)methanol is prepared as described in J. Org. Chem. 1994, 59, 7701-7703.

### Precursor 3: Synthesis of bis(3,5-dibromophenyl)methane

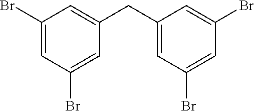

Bis(3,5-dibromophenyl)methane is prepared as described in J. Org. Chem. 1994, 59, 7701-7703.

### Precursor 4: Synthesis of bis(3,5-dicyanophenyl)ketone

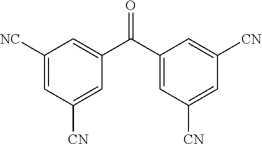

49.7 g (100 mmol) of bis(3,5-dibromophenyl)ketone, 29.4 g (250 mmol) of zinc cyanide, 2.0 g (31 mmol) of zinc dust and 8.1 g (7 mmol) of tetrakis(triphenylphosphine)palladium are heated at an internal temperature of 135° C. for seven days with vigorous stirring in 900 ml of dimethylacetamide. After cooling, 500 ml of water and 500 ml of ammonia solution (25%) are added to the mixture, which is then stirred for two hours. The solid which forms is filtered off with suction, washed five times with 200 ml of water, five times with 100 ml of methanol and subsequently taken up in dichloromethane. The solution is dried over sodium sulfate, filtered through silica gel and highly concentrated in vacuo. Addition of 200 ml of ethanol causes the precipitation of a solid, which is filtered off with suction and washed with ethanol. Recrystallisation four times from dimethylformamide leaves a colourless solid. Yield: 19.4 g (69 mmol), 68.7%, purity about 99% (HPLC).

### Precursor 5: Synthesis of bis(3,5-dicyanophenyl)methane

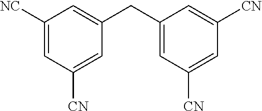

Bis(3,5-dicyanophenyl)methane is prepared analogously to precursor 4 from 38.7 g (80 mmol) of bis(3,5-dibromophenyl)methane. Yield: 15.2 g (57 mmol), 71.0%, purity about 99.5% (HPLC).

### Precursor 6: Synthesis of 3,3′,5,5′-benzophenonetetracarboxylic acid

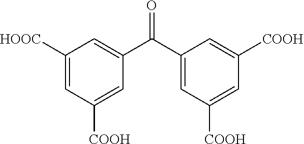

28.2 g (100 mmol) of bis(3,5-dicyanophenyl)ketone are suspended in 500 ml of ethanol, 40.0 g (1 mol) of sodium hydroxide in 500 ml of water are slowly added, and the mixture is heated under reflux for 24 hours. After cooling, the mixture is poured into 1000 ml of aqueous hydrochloric acid (5 M). The precipitate which forms is filtered off with suction, washed with water to the neutral point and subsequently washed twice with 150 ml of ethanol. Yield: 33.5 g (93.5 mmol), 93.5%, purity about 99.5% (HPLC).

### Precursor 7: Synthesis of 3,3′,5,5′-diphenylmethanetetracarboxylic acid

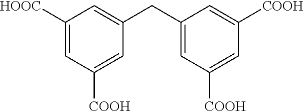

3,3′,5,5′-Diphenylmethanetetracarboxylic acid is prepared analogously to precursor 6 from 16.1 g (60 mmol) of bis(3,5-dicyanophenyl)methane. Yield: 20.0 g (58 mmol), 96.8%, purity about 99.5% (HPLC).

### Precursor 8: Synthesis of 3,3′,5,5′-benzophenonetetracarbonyl chloride

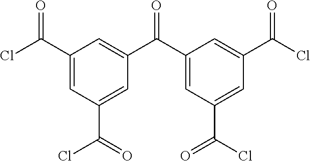

28.7 g (80 mmol) of 3,3′,5,5′-benzophenonetetracarboxylic acid are heated under reflux for four hours with 150 ml of thionyl chloride. After cooling, excess thionyl chloride is removed in vacuo. Yield: 34.2 g (79 mmol), 98.8%, purity about 99.5% (HPLC).

### Precursor 9: Synthesis of 3,3′,5,5′-diphenylmethanetetracarbonyl chloride

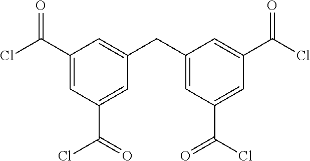

3,3′,5,5′-Diphenylmethanetetracarbonyl chloride is prepared analogously to precursor 8 from 17.2 g (50 mmol) of 3,3′,5,5′-diphenylmethanetetracarboxylic acid. Yield: 20.6 g (49 mmol), 98.6%, purity about 99.5% (HPLC).

### Precursor 10: Synthesis of 2,2-bis(3,5-dibromophenyl)-1,3-dioxolane

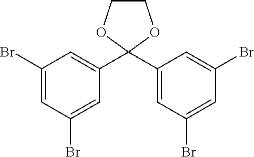

99.6 g (200 mmol) of bis(3,5-dibromophenyl)ketone are initially introduced, and 20.1 ml (300 mmol) of 2-chloroethanol are slowly added. 300 ml of dimethylformamide are then added, and the mixture is cooled to −63° C. A mixture of 33.7 g (112 mmol) of potassium tert-butoxide and 250 ml of dimethylformamide is added dropwise over the course of 40 minutes, during which a pale-brown suspension forms. After six hours, the cooling is removed. A mixture of 200 ml of saturated ammonium chloride solution and 20 ml of ammonia solution (25%) is added at an internal temperature of about −45° C. After warming to room temperature, the solid is filtered off with suction, washed once with water and three times with ethanol/water (1:1). The residue is dried three times azeotropically with toluene in a rotary evaporator, taken up in 300 ml of n-heptane, filtered off, washed with n-heptane and dried in vacuo. Yield: 100.0 g (179 mmol), 89.5%, purity about 97% (HPLC).

### Precursor 11: Synthesis of m-terphenyl-3-boronic acid

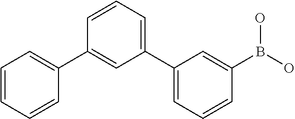

m-Terphenyl-3-boronic acid is prepared as described in WO 07/043,357.

### Precursor 12: Synthesis of m-quaterphenyl-3-boronic acid

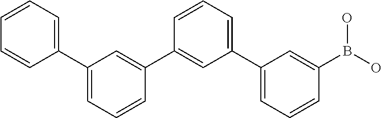

m-Quaterphenyl-3-boronic acid is prepared in accordance with m-terphenyl-3-boronic acid, as described in WO 07/043357.

### Precursor 13: 2-Chloro-4,6-diphenyl-1,3,5-triazine

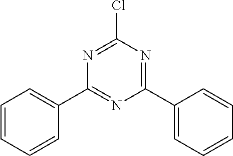

Preparation according to EP 810453. Analogous 2-chloro-4,6-diaryl-1,3,5-triazines (aryl=4-biphenyl, 3-biphenyl, 3-m-terphenyl, 3,5-diphenylphen-1-yl) are likewise obtained by this process.

### Example 1

### Synthesis of bis(3,5-diphenylphenyl)ketone

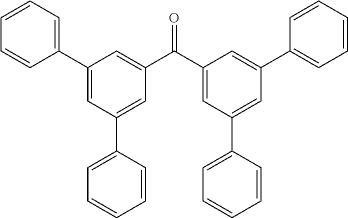

74.7 g (150 mmol) of bis(3,5-dibromophenyl)ketone, 109.7 g (900 mmol) of phenylboronic acid, 267.5 g (1162 mmol) of tripotassium phosphate monohydrate, 5.5 g (18 mmol) of tri-o-tolylphosphine and 673.5 mg (3 mmol) of palladium(II) acetate are suspended in a mixture of 600 ml of toluene, 300 ml of dioxane and 750 ml of water and heated under reflux for 72 h. After cooling, the organic phase is separated off, washed three times with water and dried over sodium sulfate. The mixture is subsequently filtered through aluminium oxide and concentrated to about 200 ml, and 500 ml of ethanol are added, during which the crude product precipitates out. The solid is filtered off with suction and washed with 100 ml of ethanol, then dissolved five times in boiling toluene and re-precipitated at elevated temperature by addition of ethanol. Yield: 44.0 g (90 mmol), 60.2%, purity about 99.9% (HPLC).

The following compounds according to the invention (Examples 2-11) are obtained analogously to Example 1 from bis(3,5-dibromophenyl)ketone and corresponding boronic acids:

| Ex. | Boronic acid | Product | Yield |
|---|---|---|---|
| 2 | 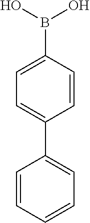 | 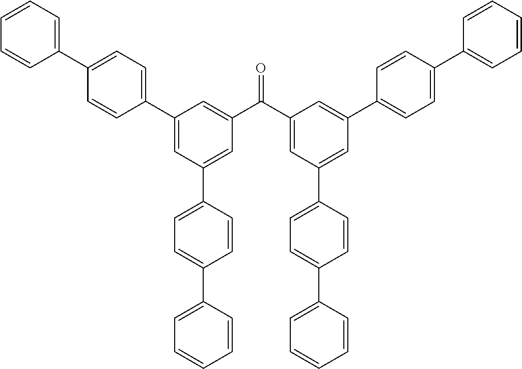 | 61.3% |
| 3 | 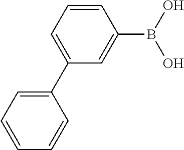 | 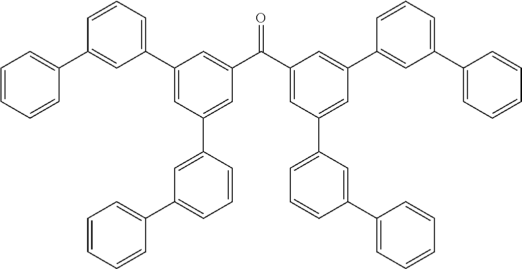 | 64.1% |
| 4 | 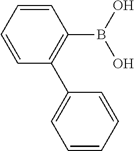 | 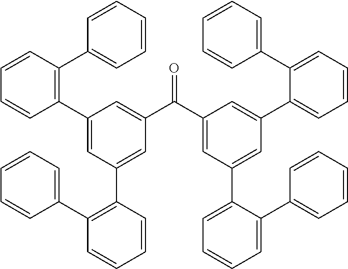 | 68.7% |
| 5 | 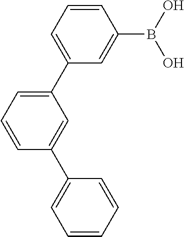 | 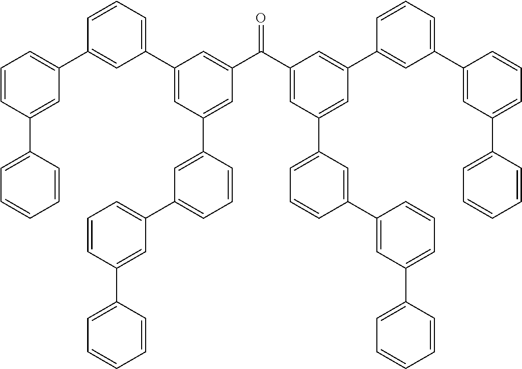 | 33.3% |
| 6 | 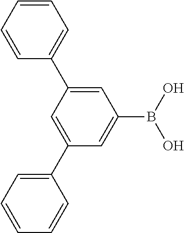 | 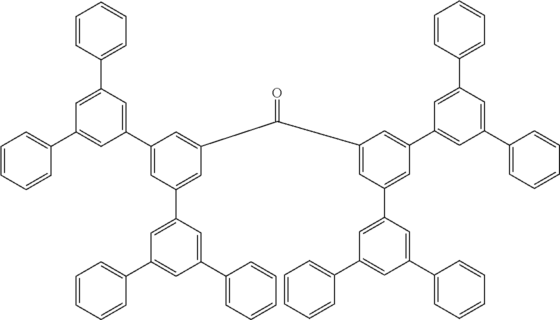 | 35.6% |
| 7 | 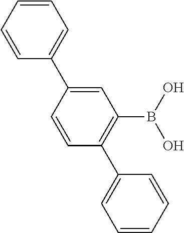 | 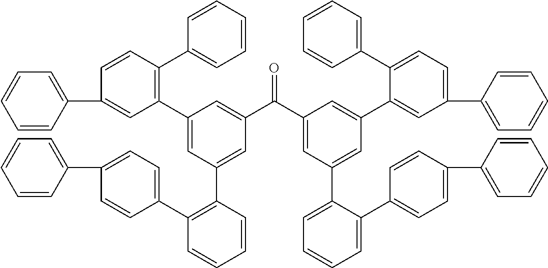 | 28.3% |
| 8 | 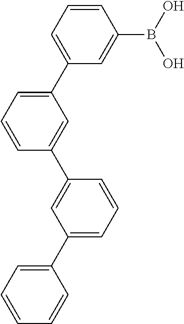 | 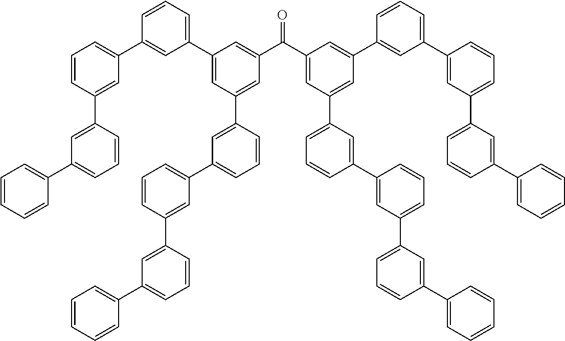 | 30.5% |
| 9 | 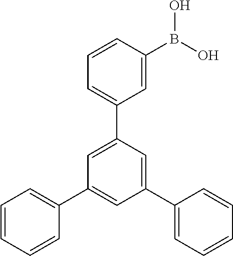 | 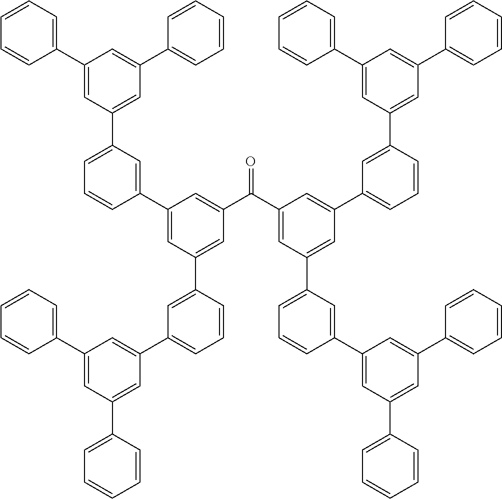 | 82.0% |
| 10 | 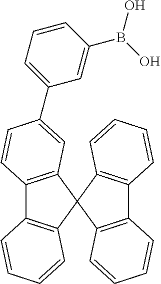 | 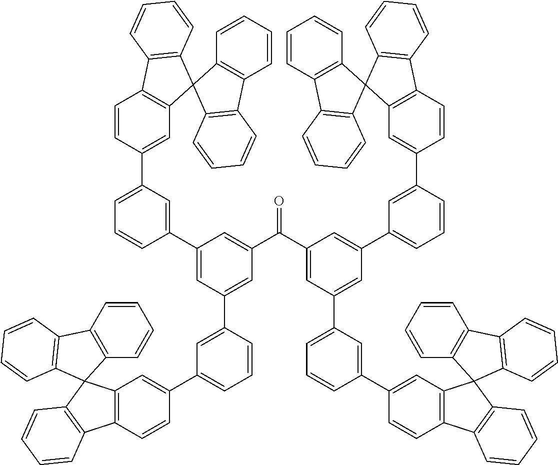 | 80.0% |
| 11 | 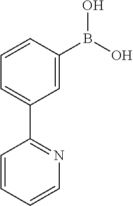 | 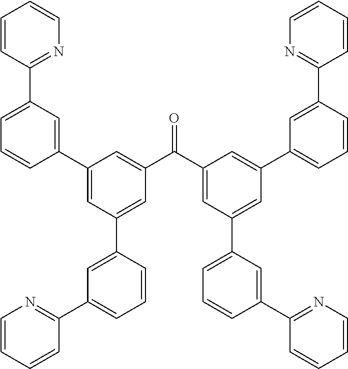 | 34.5% |

### Example 12

### Synthesis of bis(3,5-diphenylphenyl)methane

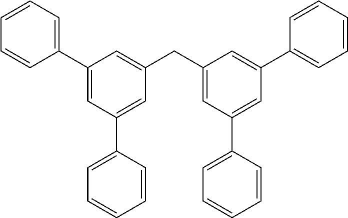

13.9 g (28 mmol) of bis(3,5-diphenylphenyl)ketone, 6.8 g (121 mmol) of potassium hydroxide and 4.9 ml (100 mmol) of hydrazine hydrate are suspended in 450 ml of diethylene glycol and heated at an internal temperature of 175° C. for five hours, during which a clear solution forms; water which forms is removed from the mixture via a water separator. On cooling to room temperature, a solid precipitates out. 400 ml of water are added, and the solid is filtered off with suction, washed with methanol, recrystallised twice from toluene and three times from dimethylformamide and subsequently dried in vacuo. Yield: 10.7 g (23 mmol), 80.9%, purity about 99.8% (HPLC).

The following compounds according to the invention (Examples 13-17) are obtained analogously to Example 12 from corresponding ketones:

| Ex. | Ketone | Product | Yield |
|---|---|---|---|
| 13 | 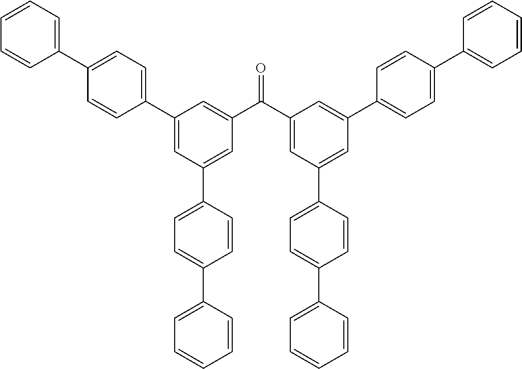 | 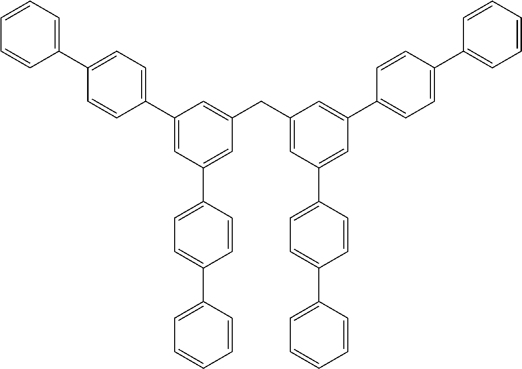 | 76.4% |
| 14 | 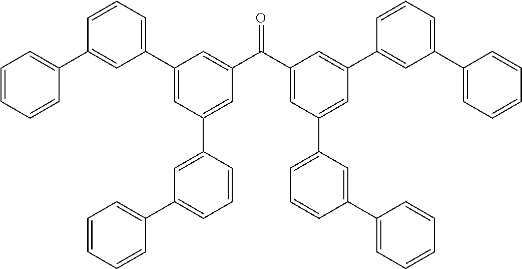 | 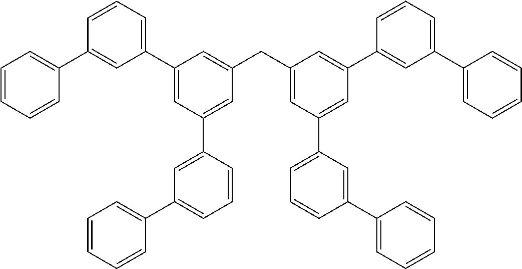 | 82.3% |
| 15 | 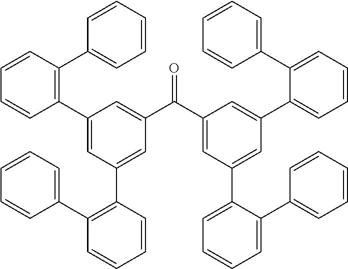 | 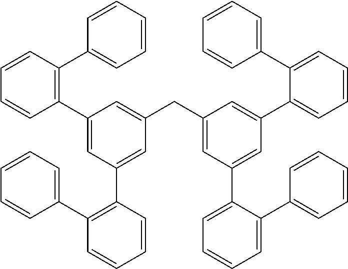 | 79.7% |
| 16 | 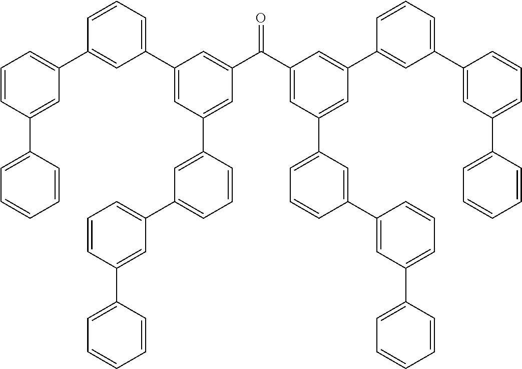 | 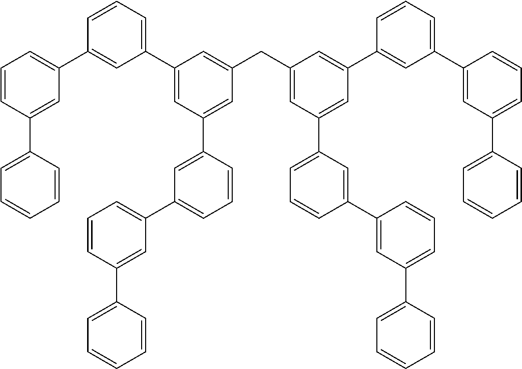 | 64.2% |
| 17 | 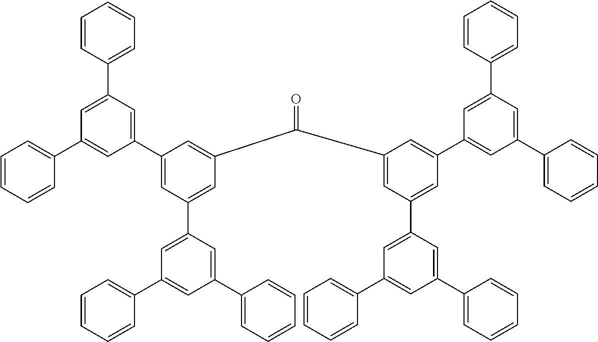 | 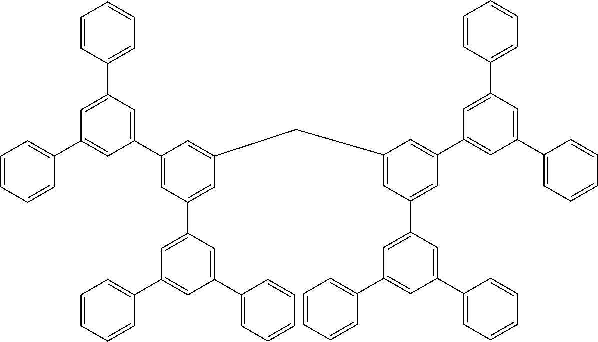 | 69.2% |

### Example 18

### Synthesis of 3,3′,5,5′-tetrabenzoylbenzophenone

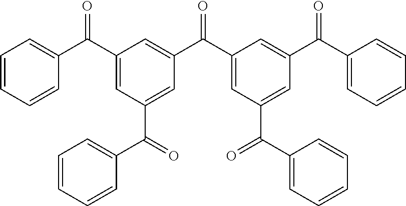

A solution of 16.3 g (30 mmol) of 2,2-bis(3,5-dibromophenyl)-1,3-dioxolane in 250 ml of tetrahydrofuran is brought to −62° C. 52 ml (130 mmol) of nbutyllithium are slowly added dropwise, and the mixture is stirred for a further two hours. 16.8 ml (165 mmol) of benzonitrile in 20 ml of tetrahydrofuran are then added dropwise, and the mixture is stirred for a further two hours and then allowed to warm to room temperature. 50 ml of 1 N aqueous hydrochloric acid are added, the mixture is boiled under reflux for 16 h, the solvent is removed in vacuo, the residue is taken up in 300 ml of dichloromethane and washed with saturated sodium hydrogencarbonate solution until neutral, and the organic phase is dried over magnesium sulfate. After removal of the dichloromethane in vacuo, the residue is chromatographed three times on silica gel (eluent heptane:ethyl acetate 3:1>1:1). Yield: 7.7 g (13 mmol), 42.9%, purity about 99.8% (HPLC).

### Example 19

### Synthesis of 3,3′,5,5′-tetrakis(1-phenylbenzimidazol-2-yl)benzophenone

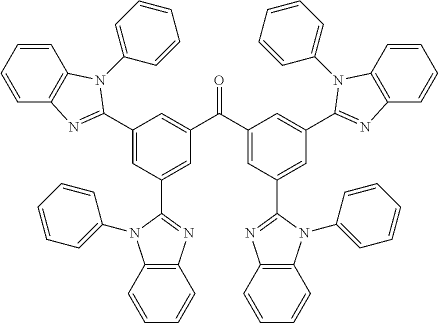

21.6 g (50 mmol) of 3,3′,5,5′-benzophenonetetracarbonyl chloride and 46.1 g (250 mmol) of 2-aminodiphenylamine are dissolved in 300 ml of dichloromethane. 78 ml (560 mmol) of triethylamine are slowly added dropwise, and the mixture is stirred for 24 hours. The organic phase is subsequently separated off, washed twice with 100 ml of water, dried over magnesium sulfate and freed from solvent in vacuo. The residue is taken up in 350 ml of ethanol and stirred for one hour. The solid is filtered off with suction and recrystallised four times from dimethylformamide. Yield: 19.9 g (21 mmol), 41.8%, purity about 99.5% (HPLC).

### Example 20

### Synthesis of 3,3′,5,5′-tetrakis(1-phenylbenzimidazol-2-yl)diphenylmethane

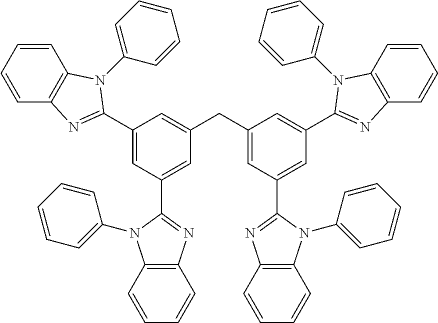

3,3′,5,5′-Tetrakis(1-phenylbenzimidazol-2-yl)diphenylmethane is prepared analogously to Example 16 from 16.7 g (40 mmol) of 3,3′,5,5′-diphenylmethanetetracarbonyl chloride. Yield: 17.8 g (19 mmol), 47.5%, purity about 99.7% (HPLC).

### Example 21

### Synthesis of bis(4,6-diphenyl-1,3,5-triazin-2-yl)ketone

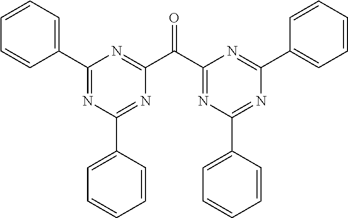

The corresponding Grignard compound is prepared from 53.5 g (200 mmol) of 2-chloro-4,6-diphenyl-1,3,5-triazine and 4.9 g (200 mmol) of magnesium, activated by means of a grain of iodine, with boiling in 700 ml of THF. The Grignard solution is cooled to −78° C., and a mixture of 9.7 g (90 mmol) of N,N-dimethylcarbamoyl chloride in 100 ml of THF is added dropwise with vigorous stirring. When the addition is complete, the mixture is stirred at −78° C. for a further 1 h, and the cooling is then removed. From an internal temperature of 0° C., a mixture of 500 ml of water and 30 ml of acetic acid is added dropwise over the course of one hour. The mixture is stirred for a further two hours and left to stand overnight. The water phase is separated off, the organic phase is dried in vacuo, 300 ml of ethanol are added to the residue, and the mixture is washed by stirring with ethanol for one hour. The solid is then filtered off with suction, washed once with 100 ml of ethanol, twice with 100 ml of ethanol/water (1:1) and twice with 100 ml of ethanol, dried in vacuo and finally recrystallised five times from DMF/BuOH. Yield: 22.0 g (45 mmol), 49.6%, purity about 99.9% (HPLC).

The following compounds according to the invention (Examples 22-25) are obtained analogously to Example 21 from corresponding 2-chloro-4,6-diaryl-1,3,5-triazines:

| Ex. | Triazine | Product | Yield |
|---|---|---|---|
| 22 | 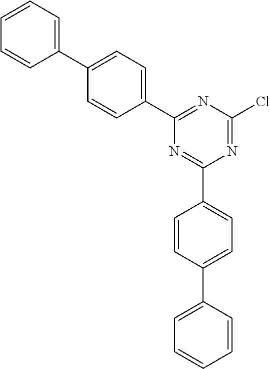 | 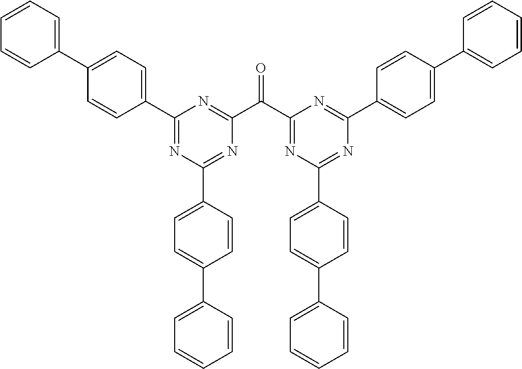 | 59.4% |
| 23 | 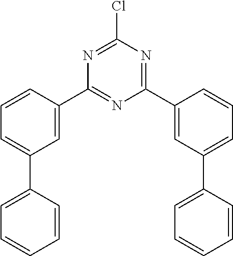 | 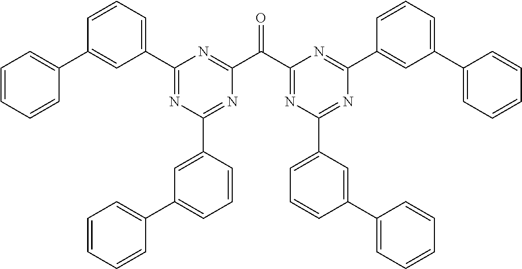 | 51.1% |
| 24 | 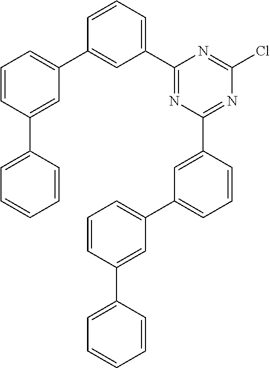 | 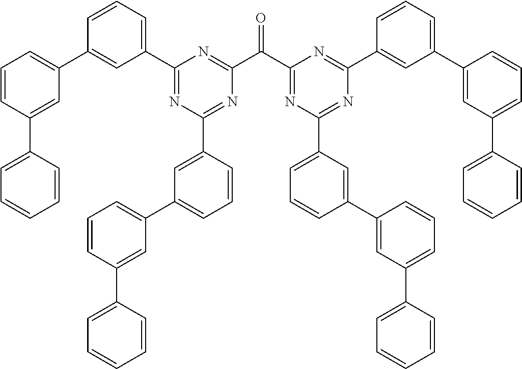 | 35.0% |
| 25 | 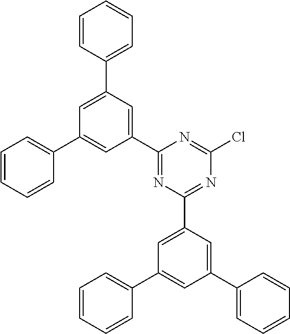 | 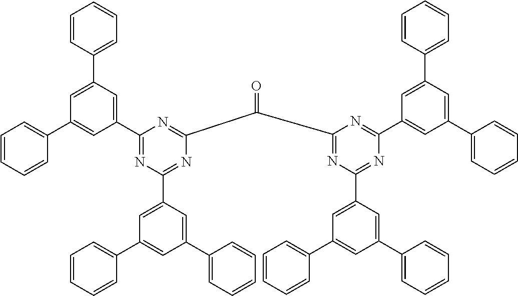 | 39.1% |

### Example 26

### Synthesis of (4,6-diphenyl-1,3,5-triazin-2-yl)-(3,6-diphenylphen-1-yl)ketone

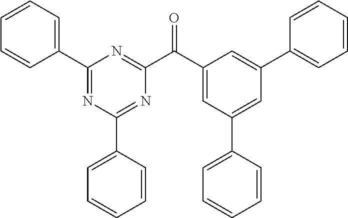

The corresponding Grignard compound is prepared from 53.5 g (200 mmol) of 2-chloro-4,6-diphenyl-1,3,5-triazine and 4.9 g (200 mmol) of magnesium, activated by means of a grain of iodine, with boiling in 700 ml of THF. The Grignard solution is cooled to 0° C., and a suspension of 48.5 g (190 mmol) of 1-cyano-3,5-diphenylbenzene in 250 ml of THF is added dropwise with vigorous stirring. When the addition is complete, the mixture is stirred at 0° C. for a further one hour, the cooling is then removed, and the mixture is stirred at room temperature for six hours and finally under reflux for 3 hours. After addition of a mixture of 200 ml of water and 100 ml of acetic acid, the mixture is heated under reflux for six hours. After cooling, the water phase is separated off, the organic phase is removed in vacuo, 300 ml of ethanol are added to the residue, and the mixture is washed by stirring with ethanol for one hour. The solid is then filtered off with suction, washed once with 100 ml of ethanol, twice with 100 ml of ethanol/water (1:1) and twice with 100 ml of ethanol, dried in vacuo and finally recrystallised five times from acetone/EtOH. Yield: 55.3 g (113 mmol), 59.5%, purity about 99.9% (HPLC).

The following compounds according to the invention (Examples 27-30) are obtained analogously to Example 26 from corresponding 2-chloro-4,6-diaryl-1,3,5-triazines:

| Ex. | Triazine | Product | Yield |
|---|---|---|---|
| 27 | 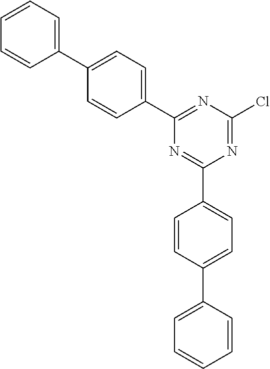 | 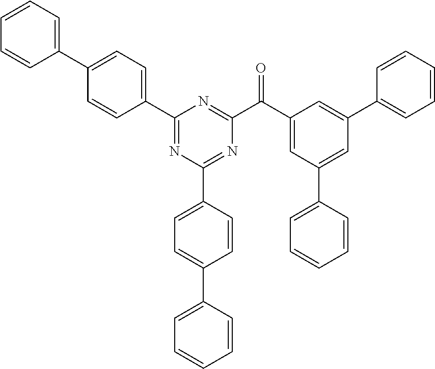 | 41.7% |
| 28 | 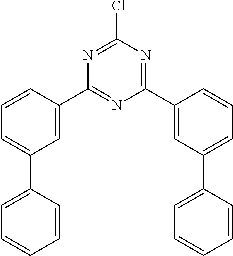 | 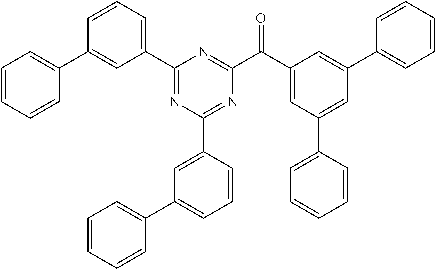 | 33.2% |
| 29 | 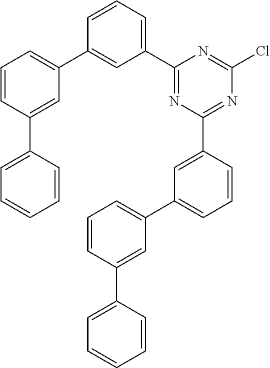 | 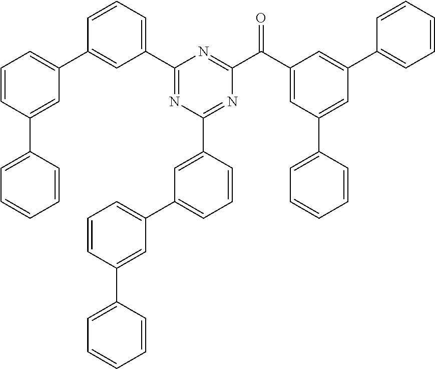 | 27.0% |
| 30 | 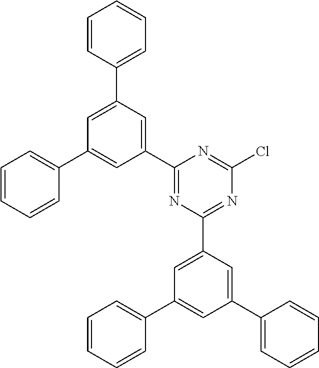 | 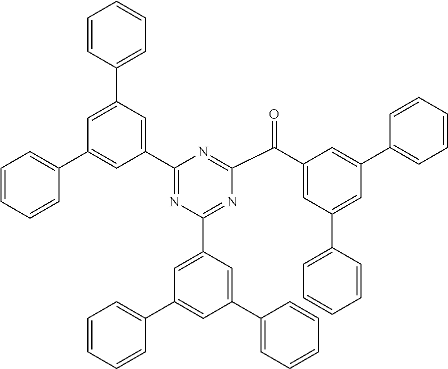 | 35.8% |

### Example 31

### Synthesis of bis-1,3-(diphenylaminophenyl)ketone

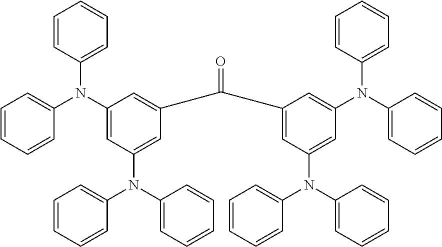

607 mg (3 mmol) of tri-tert-butylphosphine and then 337 mg (1.5 mmol) of palladium(II) acetate are added to a suspension of 37.3 g (75 mmol) of bis(3,5-dibromophenyl)ketone, 76.2 g (450 mmol) of diphenylamine and 49.0 g (510 mmol) of sodium tert-butoxide in 1000 ml of toluene, and the mixture is subsequently heated under reflux for 3 h. After cooling, 500 ml of water are added, the mixture is filtered through a short silica-gel column, and the organic phase is separated off, washed three times with 500 ml of water, dried over magnesium sulfate and subsequently evaporated virtually to dryness in vacuo. The residue is taken up in 1000 ml of warm ethanol. After cooling, the precipitated solid is filtered off with suction, dried in vacuo and recrystallised five times from DMF. Yield: 23.8 g (28 mmol), 37.7%, purity about 99.9% (HPLC).

The following compounds according to the invention (Examples 32-37) are obtained analogously to Example 31 from corresponding amines:

| Ex. | Amine | Product | Yield |
|---|---|---|---|
| 32 | 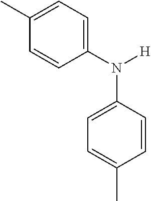 | 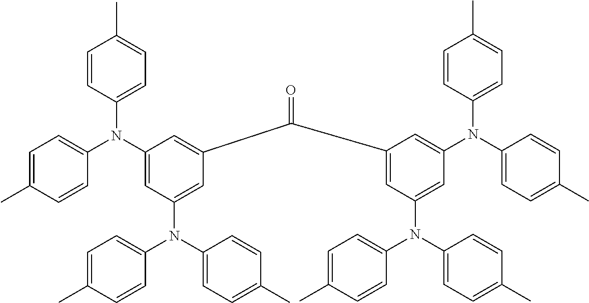 | 41.5% |
| 33 | 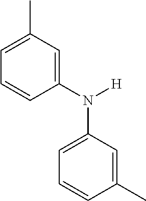 | 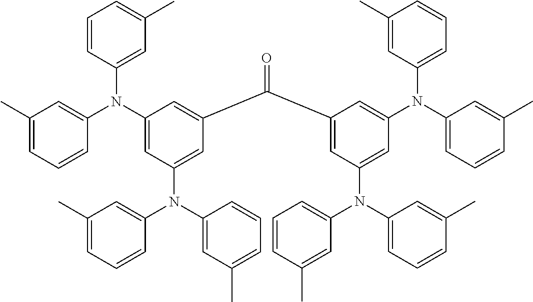 | 32.7% |
| 34 | 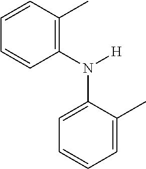 | 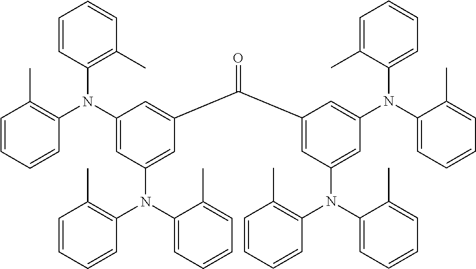 | 37.6% |
| 35 | 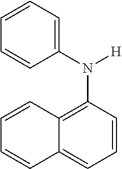 | 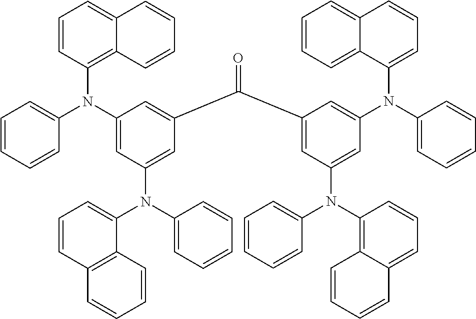 | 46.4% |
| 36 | 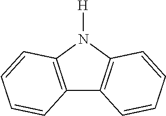 | 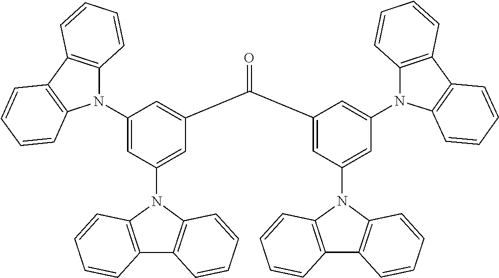 | 43.5% |
| 37 | 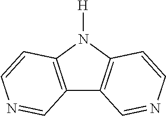 | 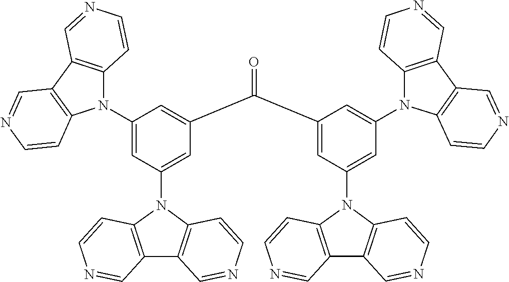 | 44.3% |

### Example 38

### Production and characterisation of organic electroluminescent devices

Electroluminescent devices according to the invention can be produced as described, for example, in WO 05/003253. The results for various OLEDs are compared here. The basic structure, the materials used, the degree of doping and the layer thicknesses thereof are identical for better comparability. The first four device examples describe comparative standards in accordance with the prior art, in which the emission layer consists of the host material (or matrix) bis(9,9′-spirobifluoren-2-yl)ketone (SK) and various guest materials (dopants) TEG for green emission or TER for red emission or TEB for blue emission. Furthermore, OLEDs of various structures are described. OLEDs having the following structure are produced analogously to the general process mentioned above:

| | |
|---|---|
| Hole-injection layer (HIL) | 20 nm of 2,2′,7,7′-tetrakis(di-para-tolyl- |
| | amino)spiro-9,9′-bifluorene |
| Hole-transport layer (HTL) | 20 nm of NPB (N-naphthyl-N-phenyl-4,4′- |
| | diaminobiphenyl) |
| Electron-blocking layer | 15 nm of EBL-1 (9,9-bis(3,5- |
| (EBL, optional) | diphenylaminophenyl)fluorene) |
| Emission layer (EML) | 40 nm of host material: spiroketone (SK) |
| | (bis(9,9′-spirobifluoren-2-yl) ketone) as |
| | comparison or compounds according to |
| | the invention. The host material may also |
| | consist of a mixture of two materials. |
| | Dopant: 10% by vol. doping; compounds |
| | see below |
| Hole-blocking layer | 10 nm of compound according to the |
| (HBL, optional) | invention (see Table 1) |
| Electron conductor (ETL) | 20 nm of AIQ₃ (tris(quinolinato)aluminium |
| | (III)) as comparison |
| Cathode | 1 nm of LiF, 100 nm of Al on top |

The structures of EBL-1, TEG-1 (synthesised in accordance with WO 04/085449), TEG-2 and TEG-3 (each synthesised in accordance with US 2001/0019782), TER-1, TER-2, TEB-1, SK, LSK, CBZ and TCTA are depicted below for clarity.

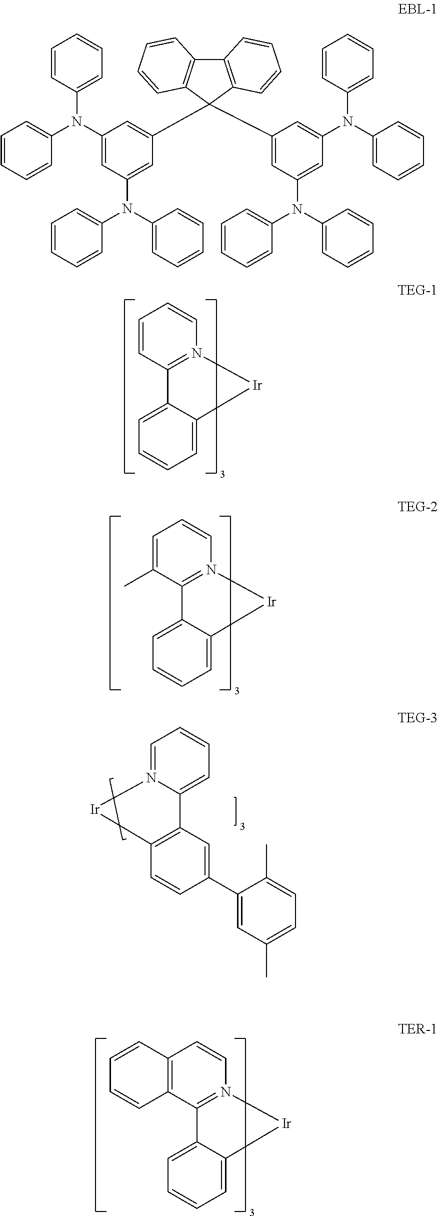

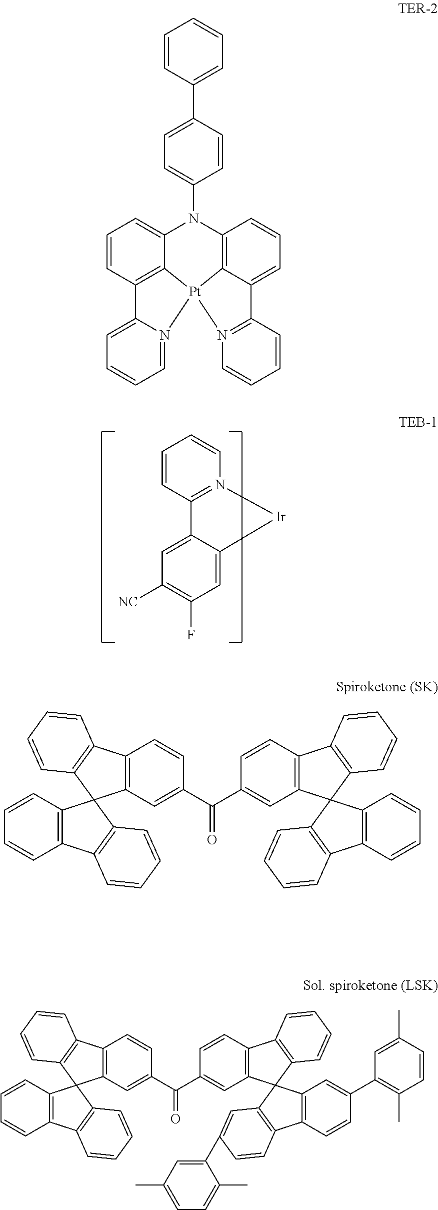

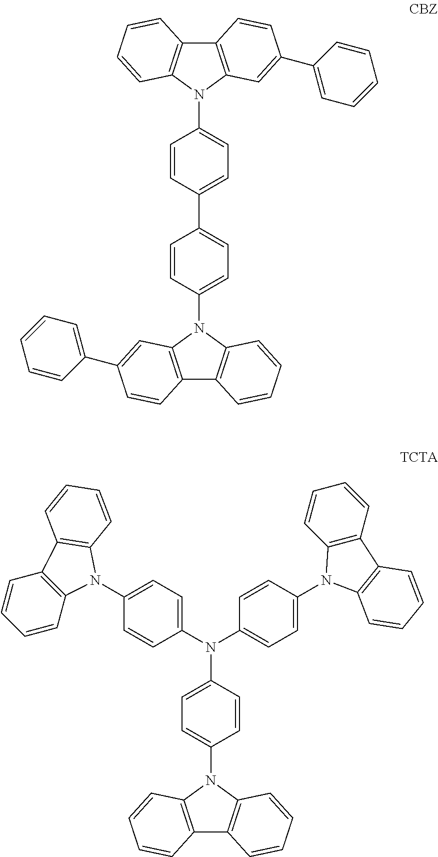

Compounds 1 to 6 according to the invention are shown below:

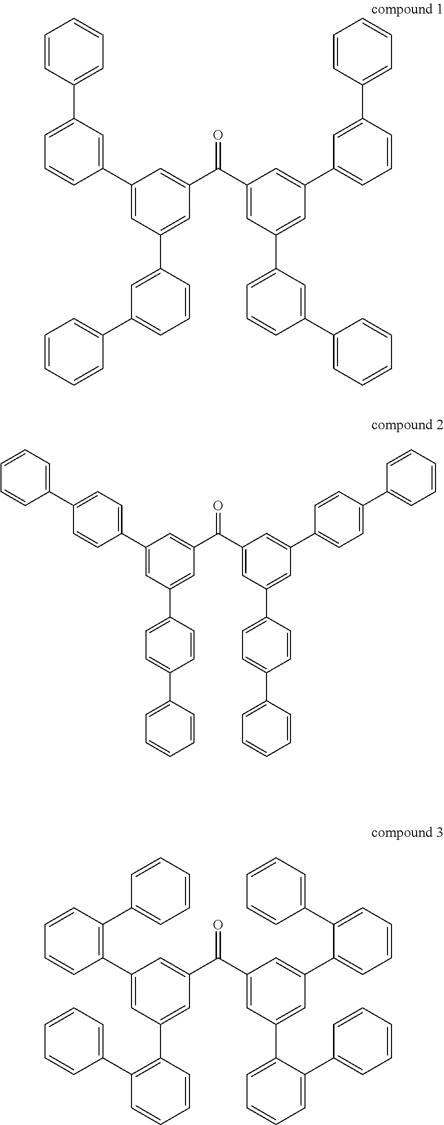

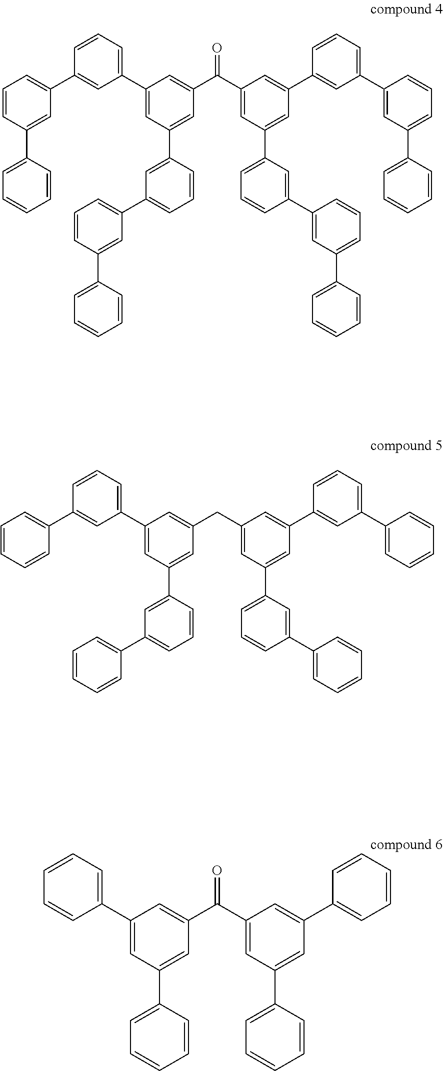

These as yet unoptimised OLEDs are characterised by standard methods; the electroluminescence spectra, the efficiency (measured in cd/A) as a function of the luminance, calculated from current-voltage-luminance characteristic lines (IUL characteristic lines), and the lifetime are determined for this purpose.

As evident from Table 1, electroluminescent devices comprising the green- or red-emitting dopants (TEG and TER) exhibit superior behaviour in the measured efficiencies, voltages, colour and lifetime compared with the comparative devices comprising the host material SK.

Table 2 shows corresponding values for the blue dopant TEB-1. The host material SK is unsuitable for blue dopants. Extremely weak electroluminescence is observed with this material, meaning that the values for efficiency and voltage cannot be determined in a meaningful way.

TABLE 1
Device results with compounds according to the invention with
TEG-1, TEG-2, TER-1 and TER-2 as dopants

| | | Max. | | | Lifetime [h], |
|---|---|---|---|---|---|
| | EML | eff. | Voltage [V] | CIE | initial luminance |
| Ex. | 40 nm | [cd/A] | at 1000 cd/m² | (x, y) | 1000 cd/m² |
| 39 | SK:TEG-1 | 30 | 4.4 | 0.38/0.57 | 8000 |
| comp. | | | | | |
| 40 | SK:TEG-2 | 33 | 4.3 | 0.39/0.57 | 12000 |
| comp. | | | | | |
| 41 | SK:TER-1 | 6 | 4.2 | 0.69/0.31 | 30000 |
| comp. | | | | | |
| 42 | SK:TER-2 | 5.4 | 5.6 | 0.66/0.34 | 15000 |
| comp. | | | | | |
| 43 | Comp. 1:TEG-1 | 37 | 4.0 | 0.34/0.61 | 21000 |
| 44 | Comp. 1:TEG-2 | 40 | 3.9 | 0.34/0.61 | 25000 |
| 45 | Comp. 1:TER-1 | 6.1 | 4.1 | 0.69/0.31 | 40000 |
| 46 | Comp. 1:TER-2 | 7 | 6.3 | 0.66/0.34 | 14000 |
| 47 | EBL-1/Comp. 1:TEG-1 | 49 | 4.4 | 0.34/0.61 | 22000 |
| 48 | EBL-1/Comp. 1:TEG-2 | 51 | 4.3 | 0.34/0.60 | 28000 |
| 49 | Comp. 2:TER-2 | 10.5 | 6.6 | 0.66/0.34 | 17000 |
| 50 | Comp. 3:TEG-1 | 38 | 4.4 | 0.32/0.62 | 12000 |
| 51 | Comp. 1:TEG-1 30 nm/ | 37 | 4.0 | 0.34/0.61 | 22000 |
| | (HBL = Comp. 1 10 nm) | | | | |
| 52 | Comp. 4:TEG-1 30 nm/ | 35 | 4.5 | 0.32/0.61 | 12000 |
| | (HBL = Comp. 1 10 nm) | | | | |
| 53 | EBL-1/Comp. 1:Comp. | 34.3 | 4.5 | 0.32/0.62 | 15000 |
| | 5:TEG-1 30 nm/ | | | | |
| | (HBL = Comp. 1 10 nm) | | | | |
| 54 | SK:CBZ:TEG-1 | 36 | 4.7 | 0.34/0.60 | 14000 |
| comp. | | | | | |
| 55 | Comp. 1:CBZ:TEG-1 | 40 | 4.7 | 0.32/0.62 | 25000 |
| 56 | SK:TCTA:TEG-1 | 38 | 3.7 | 0.35/0.60 | 3000 |
| comp. | | | | | |
| 57 | Comp. 1:TCTA:TEG-1 | 42 | 3.7 | 0.32/0.62 | 6000 |

TABLE 2
Device results with compounds according to the invention with
TEB-1 as dopant

| | | Max. | | |
|---|---|---|---|---|
| | EML | eff. | Voltage [V] | CIE |
| Ex. | 40 nm | [cd/A] | at 1000 cd/m² | (x, y) |
| 58 | SK:TEB-1 | — | — | — |
| comp. | | | | |
| 59 | EBL-1/Comp. 1:TEB-1 | 13 | 8.2 | 0.17/0.28 |
| 60 | EBL-1/Comp. 4:TEB-1 | 16 | 9.6 | 0.16/0.27 |
| 61 | EBL-1/Comp. 6:TEB-1 | 17 | 8.5 | 0.17/0.27 |
| 62 | EBL-1/Comp. 1:Comp. | 16 | 9.3 | 0.15/0.26 |
| | 5:TEB-1 | | | |
| | (HBL = Comp. 1 10 nm) | | | |

### Example 63

### Production and Characterisation of Organic Electroluminescent Devices from Solution

The materials according to the invention can also be used from solution, where they result in significantly simpler devices which still have good properties. The production of such components is based on the production of polymeric light-emitting diodes (PLEDs), which has already been described a number of times in the literature (for example in WO 2004/037887). In the present case, the compounds according to the invention or likewise soluble comparative compounds (LSK) are dissolved in toluene or chlorobenzene together with the triplet emitter TEG-3. The structures of the materials used are depicted above in Example 38. The typical solids content of such solutions is between 16 and 25 g/l if, as here, the layer thickness of 80 nm which is typical for a device is to be achieved by means of spin coating. The device here has the following structure:

ITO anode/80 nm buffer layer comprising PEDOT/20 nm interlayer/80 nm emitting layer comprising 17% by weight of TEG-3 in the corresponding matrix material (see Table 3)/cathode comprising 3 nm of Ba and 150 nm of Al.

Structured ITO substrates and the material for the so-called buffer layer (PEDOT, actually PEDOT:PSS) are commercially available (ITO from Technoprint and others, PEDOT:PSS as aqueous dispersion Clevios Baytron P from H.C. Starck). The interlayer used serves for hole injection; in this case, HIL-012 from Merck is used. The emission layer is applied by spin coating in an inert-gas atmosphere, argon in the present case, and dried by heating at 120° C. for 10 min. Finally, a barium/aluminium cathode is applied by vacuum vapour deposition. The hole-blocking and/or electron-transport layers used in the above examples can also be applied by vapour deposition between the emitting layer and the cathode, and the interlayer may also be replaced by one or more layers which only have to satisfy the condition of not being detached again by the subsequent processing step of deposition of the emitting layer from solution.

The solution-processed devices are also characterised by standard methods. Table 3 summarises the data obtained. It is also apparent in the area of solution-processed devices that the materials according to the invention are superior to those previously available in terms of efficiency and lifetime.

TABLE 3
Results with materials processed from solution

| | | Max. | | | |
|---|---|---|---|---|---|
| | EML | eff. | Voltage [V] | CIE | Lifetime [h], initial |
| Ex. | 40 nm | [cd/A] | at 1000 cd/m² | (x, y) | luminance 1000 cd/m² |
| 64 | LSK:TEG-3 | 22 | 5.9 | 0.35/0.61 | 3000 |
| comp. | | | | | |
| 65 | Comp. 1:TEG-3 | 28 | 6.8 | 0.35/0.61 | 3500 |
| 66 | Comp. 3:TEG-3 | 28 | 6.7 | 0.31/0.63 | NA |
| 67 | LSK:Comp. 5:TEG-3 | 31 | 5.5 | 0.33/0.62 | 3000 |
| 68 | LSK:CBZ:TEG-3 | 29 | 6.0 | 0.35/0.61 | 9500 |
| comp. | | | | | |
| 69 | Comp. 1:CBZ:TEG-3 | 34 | 7.5 | 0.33/0.63 | 14000 |
| 70 | LSK:TEG-3 | 21 | 7.4 | 0.36/0.61 | 100 |
| comp. | (+ vapour-deposited | | | | |
| | ETL) | | | | |
| 71 | Comp. 1:TEG-3 | 30 | 5.5 | 0.34/0.62 | 8000 |
| | (+ vapour-deposited | | | | |
| | ETL) | | | | |

1.-15. (canceled)

16. A compound of the formula (1) 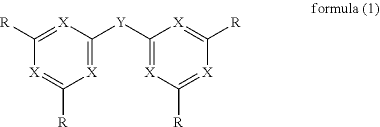
where the following applies to the symbols used:
Y is C═O or C(R¹)₂;
X is on each occurrence, identically or differently, CR² or N;
R is on each occurrence, identically or differently, an aromatic or heteroaromatic ring system having 5 to 60 aromatic ring atoms, which is optionally substituted by one or more radicals R³, or an N(Ar)₂, Si(Ar)₃, C(═O)Ar, OAr, ArSO, ArSO₂, P(Ar)₂, P(O)(Ar)₂ or B(Ar)₂ group;
Ar is on each occurrence, identically or differently, an aromatic or heteroaromatic ring system having 5 to 30 aromatic ring atoms, which is optionally substituted by one or more non-aromatic radicals R³; two radicals Ar here which are bonded to the same nitrogen, phosphorus or boron atom is optionally linked to one another by a single bond or a bridge selected from B(R⁴), C(R⁴)₂, Si(R⁴)₂, C═O, C═NR⁴, C═C(R⁴)₂, O, S, S═O, SO₂, N(R⁴), P(R⁴) and P(═O)R⁴;
R¹ is on each occurrence, identically or differently, H, D, F or a linear alkyl group having 1 to 20 C atoms or a branched or cyclic alkyl group having 3 to 20 C atoms; a plurality of radicals R¹ here optionally forms a ring system with one another;
R² is on each occurrence, identically or differently, H, D, F, CN, a straight-chain alkyl, alkoxy or thioalkoxy group having 1 to 40 C atoms or a branched or cyclic alkyl, alkoxy or thioalkoxy group having 3 to 40 C atoms, each of which is optionally substituted by one or more radicals R⁴, where one or more non-adjacent CH₂ groups is optionally replaced by R⁴C═CR⁴, C≡C, O or S and where one or more H atoms is optionally replaced by F;
R³ is on each occurrence, identically or differently, H, D, F, Cl, Br, I, CHO, N(Ar)₂, C(═O)Ar, P(═O)(Ar)₂, S(═O)Ar, S(═O)₂Ar, CR²═CR²Ar, CN, NO₂, Si(R⁴)₃, B(OR⁴)₂, B(R⁴)₂, B(N(R⁴)₂)₂, OSO₂R⁴, a straight-chain alkyl, alkoxy or thioalkoxy group having 1 to 40 C atoms or a branched or cyclic alkyl, alkoxy or thioalkoxy group having 3 to 40 C atoms, each of which is optionally substituted by one or more radicals R⁴, where one or more non-adjacent CH₂ groups is optionally replaced by R⁴C═CR⁴, C═C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴ and where one or more H atoms is optionally replaced by F, Cl, Br, I, CN or NO₂, or an aromatic or heteroaromatic ring system having 5 to 60 aromatic ring atoms, which optionally in each case is substituted by one or more radicals R⁴, or an aryloxy or heteroaryloxy group having 5 to 60 aromatic ring atoms, which is optionally substituted by one or more radicals R⁴, or a combination of these systems; two or more adjacent substituents R³ here may also form a mono- or polycyclic, aliphatic or aromatic ring system with one another;
R⁴ is on each occurrence, identically or differently, H, D or an aliphatic, aromatic and/or heteroaromatic hydrocarbon radical having 1 to 20 C atoms, in which, in addition, H atoms is optionally replaced by F; two or more adjacent substituents R⁴ here optionally forms a mono- or polycyclic, aliphatic or aromatic ring system with one another;
the following compounds are excluded from the invention:

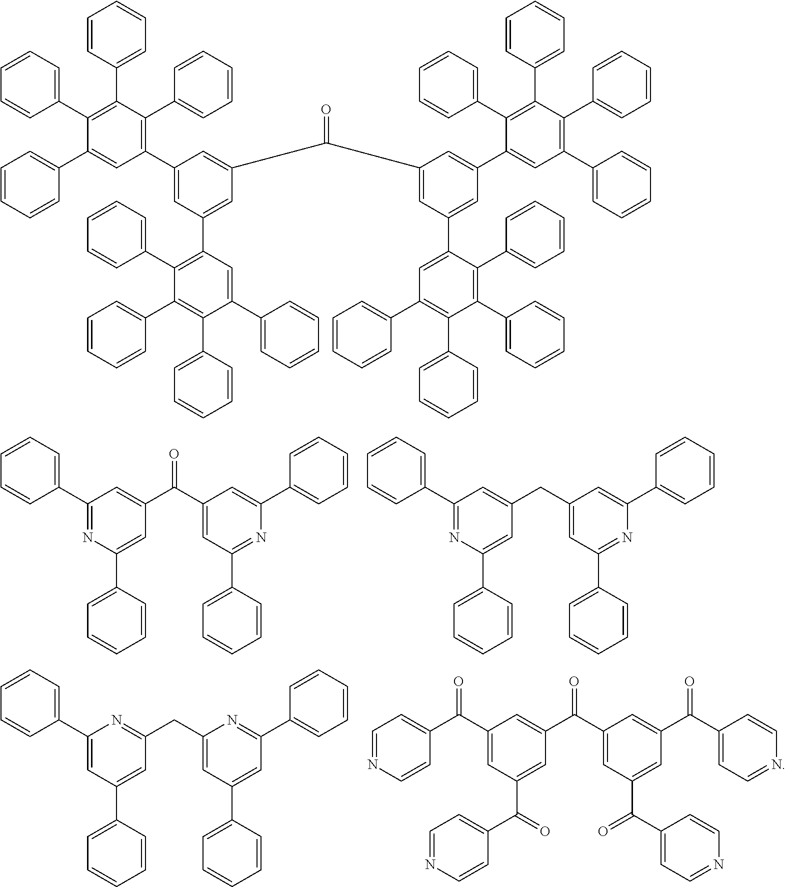

17. The compound according to claim 16, wherein all symbols X in a cyclic system either stands for CR² or all symbols X in a cyclic system stand for N.

18. The compound according to claim 16, wherein the symbol R stands, identically or differently on each occurrence, for an aromatic or heteroaromatic ring system having 5 to 30 aromatic ring atoms, which is optionally substituted by one or more radicals R³, or for an N(Ar)₂, C(═O)Ar or P(═O)Ar₂ group.

19. The compound according to claim 16, wherein the group R is phenyl, o-biphenyl, m-biphenyl, p-biphenyl, o-terphenyl, m-terphenyl, p-terphenyl, 3,5-(diphenyl)phenyl, m-quaterphenyl, 2-fluorenyl, 2-spirobifluorenyl, 1-naphthyl, 2-naphthyl, 1-, 2- or 9-anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, 2-, 4- or 5-pyrimidinyl, 1,3,5-triazinyl, in particular substituted by aromatic groups, N-benzimidazolyl, phenyl-N-benzimidazolyl, N-phenylbenzimidazolyl, phenyl-N-phenylbenzimidazolyl, thiophene, oxazole, oxadiazole, thiadiazole or benzothiazole, where these groups may each be substituted by one or more substituents R³.

20. The compound according to claim 16, wherein the group R is selected from structures of the formulae (2) to (16), where the dashed bond in each case indicates the linking of this unit and where the groups may each be substituted by one or more radicals R³:

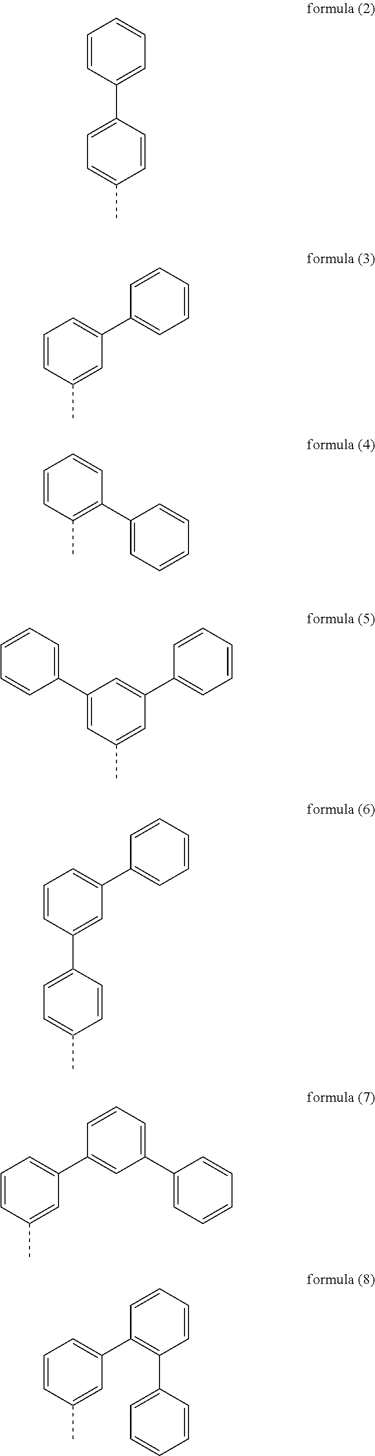

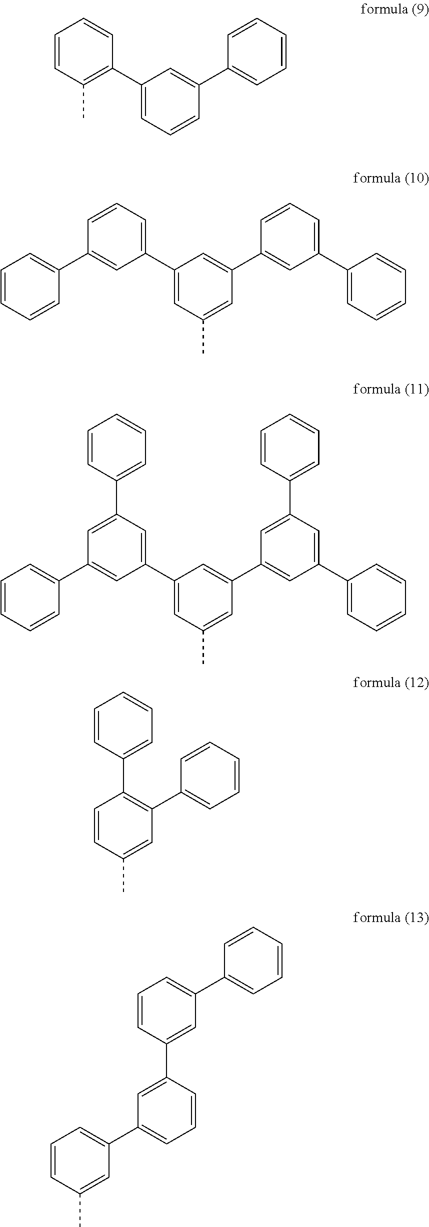

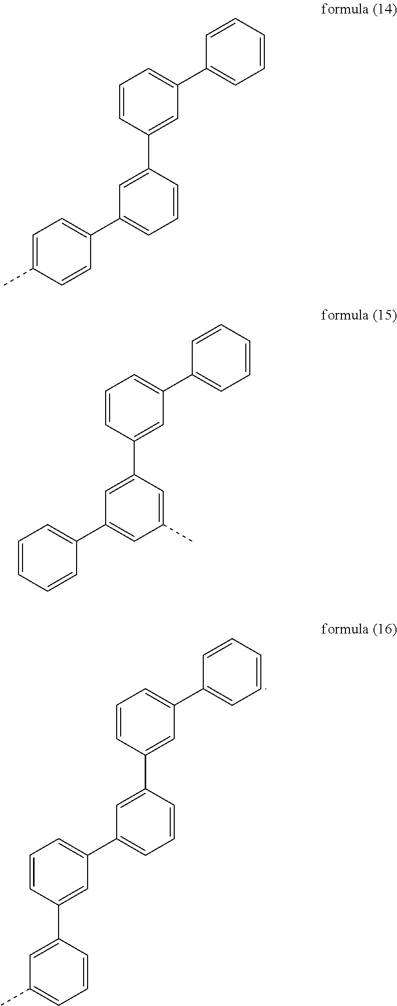

21. The compound according to claim 16, wherein the radical R is selected from the groups of the formula (17) or formula (18): 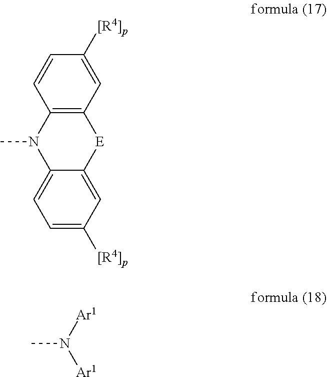
wherein
R⁴ is on each occurrence, identically or differently, H, D or an aliphatic, aromatic and/or heteroaromatic hydrocarbon radical having 1 to 20 C atoms, in which, in addition, H atoms is optionally replaced by F; two or more adjacent substituents R⁴ here optionally forms a mono- or polycyclic, aliphatic or aromatic ring system with one another;
E stands for a single bond, O, S, N(R⁴) or C(R⁴)₂;
Ar¹ is, identically or differently on each occurrence, an aromatic or heteroaromatic ring system having 5 to 24 aromatic ring atoms or a triarylamine group having 15 to 30 aromatic ring atoms, each of which is optionally substituted by one or more radicals R⁴; and
p is on each occurrence, identically or differently, 0 or 1.

22. The compound according to claim 21, wherein Ar¹ is, identically or differently on each occurrence, an aryl or heteroaryl group having 6 to 14 aromatic ring atoms or a triarylamine group having 18 to 22 aromatic ring atoms, each of which is optionally substituted by one or more radicals R⁴.

23. The compound according to claim 16, wherein all groups R in compounds of the formula (1) are selected identically or in that both substituents R which are bonded to the same ring are each selected identically, but differ from the substituents R on the other ring.

24. The compound according to claim 16, selected from compounds of the formulae (19), (20) and (21): 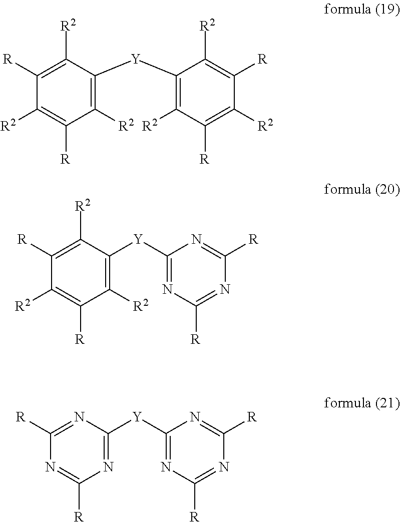
where Ar, R³ and R⁴ are as defined in claim 16, and the following applies to the other symbols used:
Y is C═O or C(R¹)₂;
R is on each occurrence, identically or differently, an aromatic or heteroaromatic ring system having 5 to 30 aromatic ring atoms;
R¹ is on each occurrence, identically or differently, H, F, a linear alkyl group having 1 to 10 C atoms, or a branched or cyclic alkyl group having 3 to 10 C atoms; a plurality of radicals R¹ optionally forms a ring system with one another;
R² is on each occurrence, identically or differently, H, F, a straight-chain alkyl group having 1 to 10 C atoms, or a branched or cyclic alkyl group having 3 to 10 C atoms.

25. The compound according to claim 24, wherein
R is on each occurrence, identically or differently and is a phenyl, o-biphenyl, m-biphenyl, p-biphenyl, o-terphenyl, m-terphenyl, p-terphenyl, 3,5-(diphenyl)phenyl, m-quaterphenyl, 1-naphthyl, 2-naphthyl, anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, N-benzimidazolyl, phenyl-N-benzimidazolyl, N-phenylbenzimidazolyl or phenyl-N-phenylbenzimidazolyl, an N(Ar)₂ group, C(═O)Ar or P(═O)Ar₂;
R¹ is on each occurrence, identically or differently, H, F, a linear alkyl group having 1 to 6 C atoms, or a branched or cyclic alkyl group having 3 to 6 C atoms; a plurality of radicals R¹ here optionally forms a ring system with one another;
R² is on each occurrence, identically or differently, H, F, a straight-chain alkyl group having 1 to 6 C atoms, or a branched or cyclic alkyl group having 3 to 6 C atoms.

26. The compound according to claim 24, wherein
R is on each occurrence, identically or differently, and is a compound of the formulae (2) to (18), C(═O)Ar or P(═O)Ar2 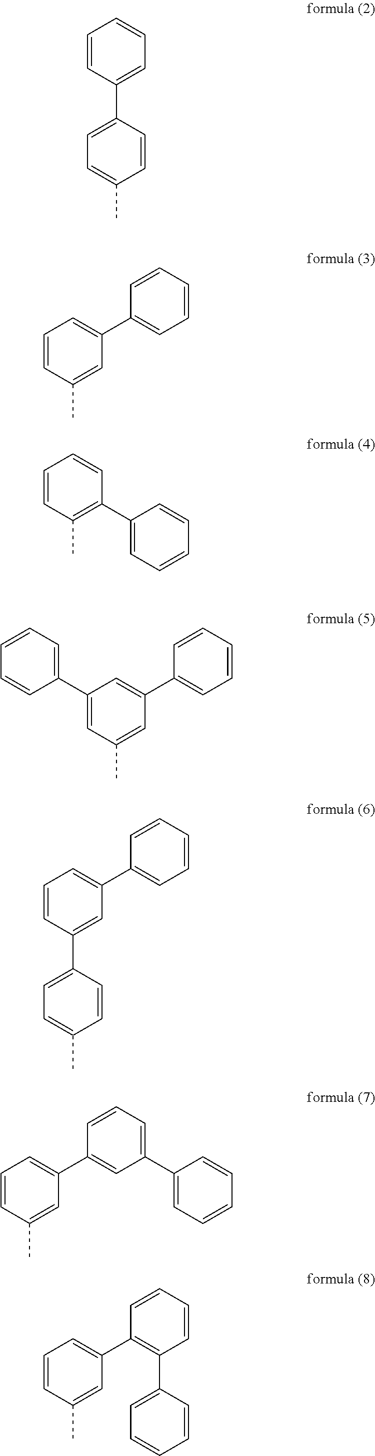 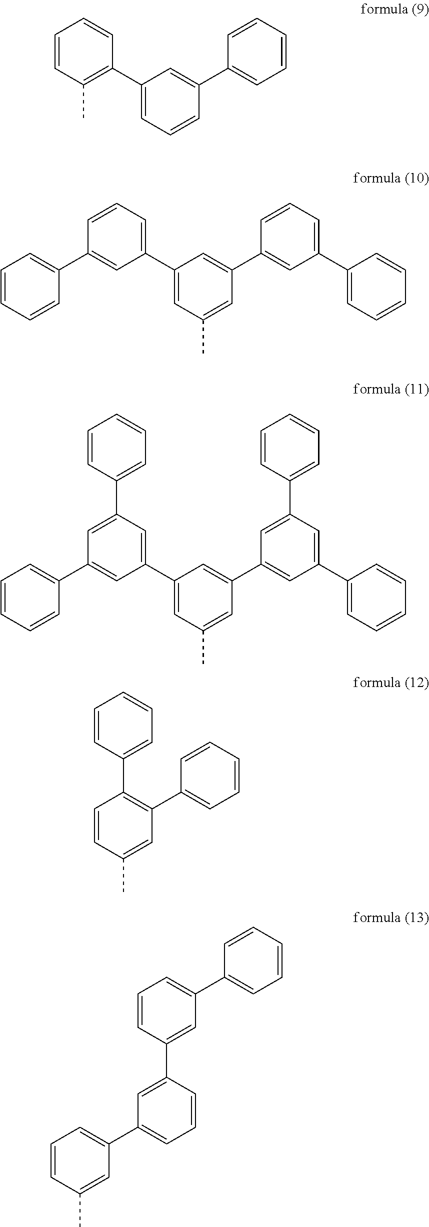 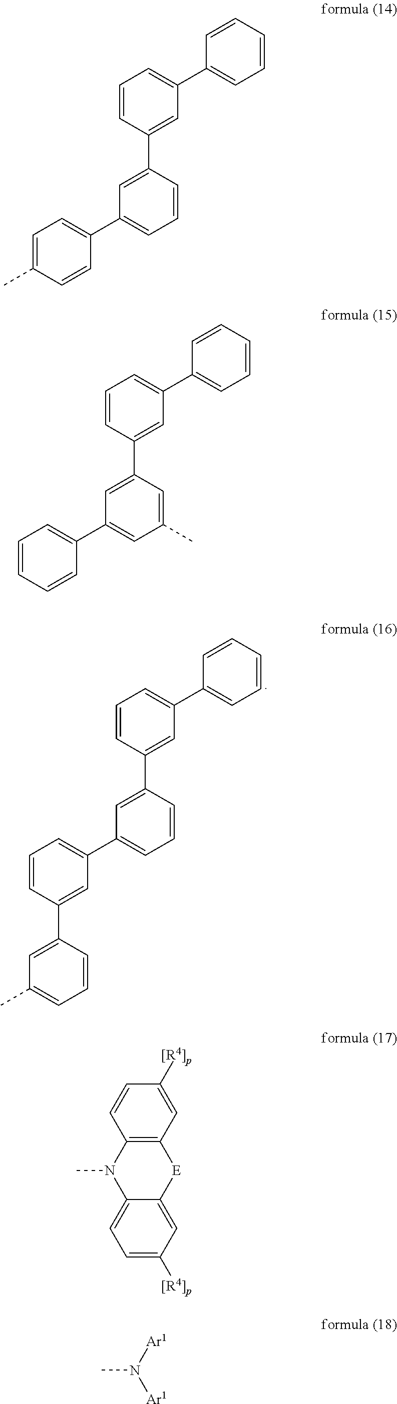
E stands for a single bond, O, S, N(R⁴) or C(R⁴)₂;
Ar¹ is, identically or differently on each occurrence, an aromatic or heteroaromatic ring system having 5 to 24 aromatic ring atoms or a triarylamine group having 15 to 30 aromatic ring atoms, each of which is optionally substituted by one or more radicals R⁴; and
p is on each occurrence, identically or differently, 0 or 1,
R¹ is on each occurrence, identically or differently, H, F, a methyl, or a branched or cyclic alkyl group having 3 to 6 C atoms; a plurality of radicals R¹ here optionally forms a ring system with one another;
R² is on each occurrence, identically or differently, H, F, or methyl.

27. The compound according to claim 16, wherein the compound of the formula (1) is of the formula (22) 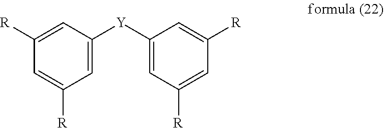
wherein
Y is C═O, CH₂, CF₂ or C (alkyl)₂, where alkyl represents an alkyl group having 1 to 6 C atoms;
R is on each occurrence, identically or differently, an aromatic or heteroaromatic ring system selected from the group consisting of phenyl, o-biphenyl, m-biphenyl, p-biphenyl, o-terphenyl, m-terphenyl, p-terphenyl, 3,5-(diphenyl)phenyl, m-quarterphenyl, 1-naphthyl, 2-naphthyl, anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, N-benzimidazolyl, phenyl-N-benzimidazolyl, N-phenylbenzimidazolyl and phenyl-N-phenylbenzimidazolyl, or an N(Ar)₂, C(═O)Ar or P(═O)Ar₂.

28. The compound according to claim 27, wherein
Y is methyl;
R is on each occurrence, identically or differently, and is a compound of the formulae (2) to (18), C(═O)Ar or P(═O)Ar2 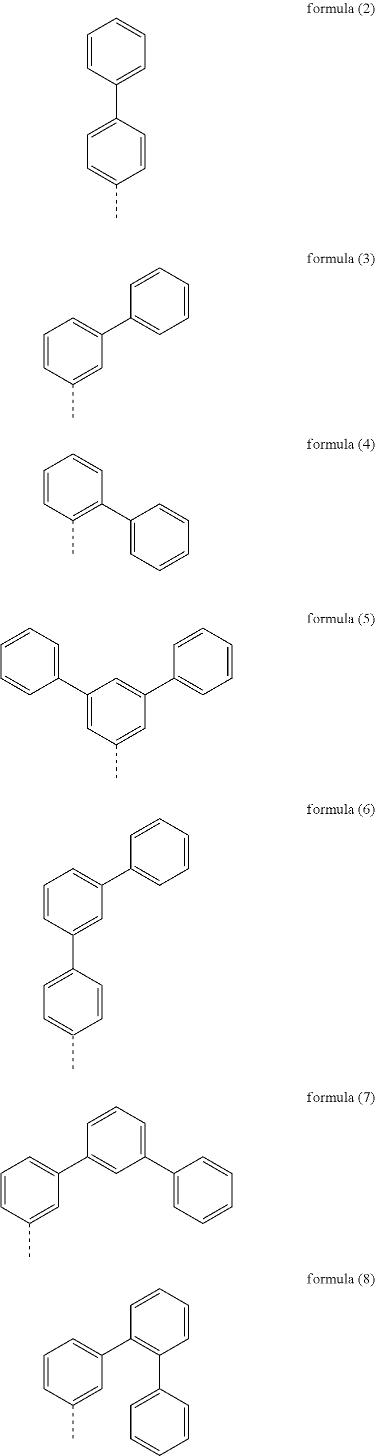 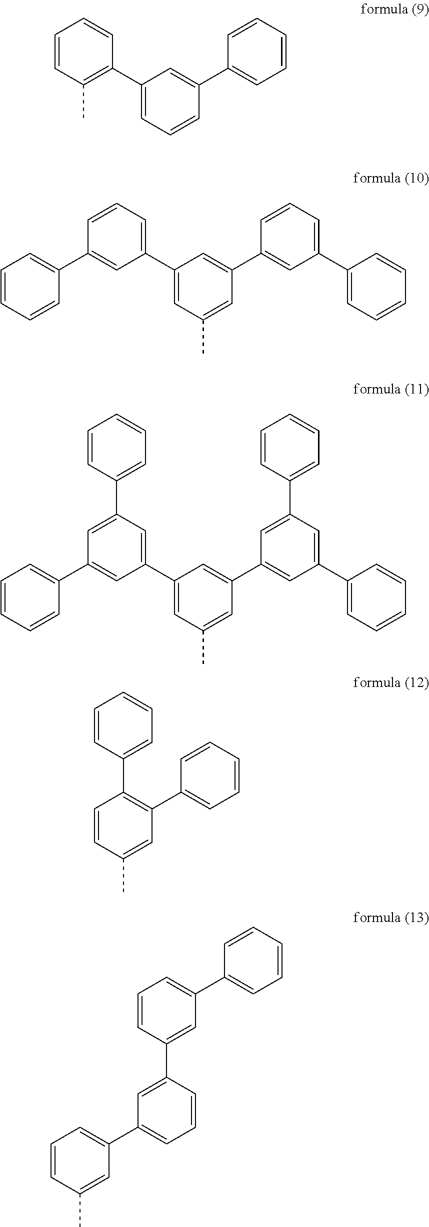 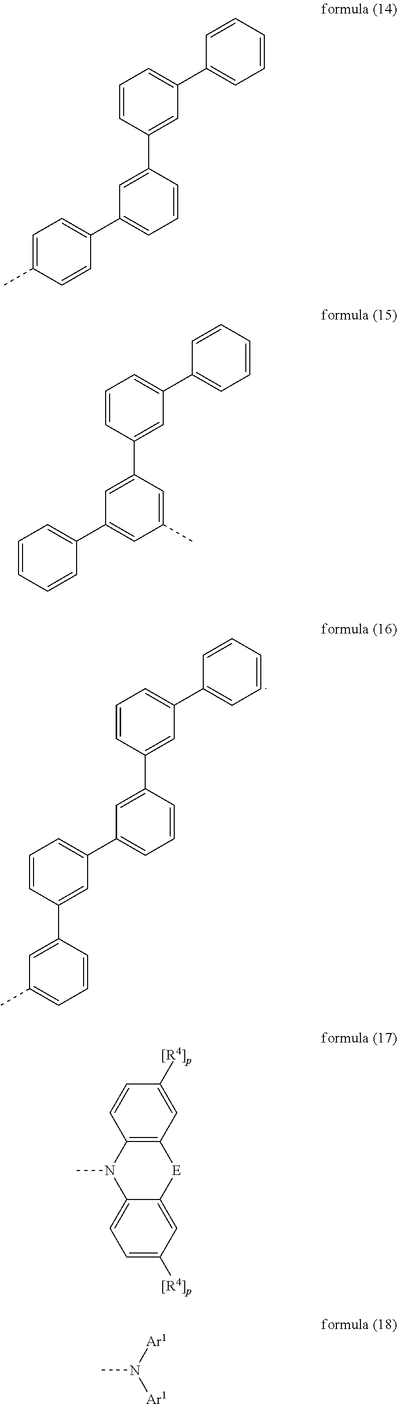
E stands for a single bond, O, S, N(R⁴) or C(R⁴)₂;
Ar¹ is, identically or differently on each occurrence, an aromatic or heteroaromatic ring system having 5 to 24 aromatic ring atoms or a triarylamine group having 15 to 30 aromatic ring atoms, each of which is optionally substituted by one or more radicals R⁴; and
p is on each occurrence, identically or differently, 0 or 1,
R¹ is on each occurrence, identically or differently, H, F, a methyl, or a branched or cyclic alkyl group having 3 to 6 C atoms; a plurality of radicals R¹ here optionally forms a ring system with one another;
R² is on each occurrence, identically or differently, H, F, or methyl.

29. The compound according to claim 16, wherein the symbol Ar stands, identically or differently on each occurrence, and is a phenyl, o-biphenyl, m-biphenyl, p-biphenyl, o-terphenyl, m-terphenyl, p-terphenyl, 3,5-(diphenyl)phenyl, m-quaterphenyl, 1-naphthyl, 2-naphthyl, anthracenyl, phenylanthracenyl, 1- or 2-naphthylanthracenyl, binaphthyl, pyrenyl, fluoranthenyl, 2-, 3-, 4-, 5-, 6- or 7-benzanthracenyl, N-benzimidazolyl, phenyl-N-benzimidazolyl, N-phenylbenzimidazolyl or phenyl-N-phenylbenzimidazolyl.

30. A process for the preparation of the compound according to claim 16, which comprises coupling a substituted or unsubstituted bis(3,5-dibromobenzophenone) to an aromatic or heteroaromatic boronic acid or a corresponding boronic acid derivative with metal catalysis or to a primary or secondary aromatic amine with metal catalysis or to a metal cyanide with metal catalysis.

31. A dimer, trimer, tetramer, pentamer, oligomer, polymer or dendrimer containing one or more compounds according to claim 16, where one or more radicals R¹ to R⁴ represent bonds between the compounds of the formula (1) in the dimer, trimer, tetramer or pentamer or bonds from the compound of the formula (1) to the polymer, oligomer or dendrimer or where this bonding takes place via substituents on the groups R.

32. A solution comprising at least one the compound according to claim 16 and at least one organic solvent.

33. A solution comprising at least one the dimer, trimer, tetramer, pentamer, oligomer or polymer according to claim 31 and at least one organic solvent.

34. An organic electronic device which comprises at least one compound according to claim 16.

35. The electronic device according to claim 34, wherein the device is selected from the group consisting of organic electroluminescent devices (OLEDs, PLEDs), organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electrochemical cells (LECs), organic laser diodes (O-lasers) or organic photoreceptors.

36. An organic electroluminescent device which comprises the compound according to claim 16, wherein the compound is employed as matrix material for a fluorescent or phosphorescent compound in an emitting layer or as hole-transport material or as hole-injection material or as electron-blocking material or as exciton-blocking material or as electron-transport material or as hole-blocking material.
